(19) United States
(12) Patent Application Publication
(10) Pub. No.: US 2016/0223532 A1
(43) Pub. Date: Aug. 4, 2016

(54) HIGH THROUGHPUT SCREENING FOR BIOMOLECULES

(71) Applicant: Agenus Inc., Lexington, MA (US)

(72) Inventor: James Andrew Rakestraw, Lexington, MA (US)

(21) Appl. No.: 15/021,255

(22) PCT Filed: Sep. 11, 2014
(86) PCT No.: PCT/US2014/055253

§ 371 Date: Mar. 10, 2016

Related U.S. Application Data
(60) Provisional application No. 61/960,143, filed on Sep. 11, 2013.

(51) Int. Cl. G01N 33/543; C12N 15/10

(52) U.S. Cl. CPC G01N 33/54313; C12N 15/1037

(57) ABSTRACT

Provided are methods and compositions for the selection of targeting moieties capable of interacting with target moieties that are displayed on a target entity, as well as methods and compositions for the selection of entities capable of inducing phenotypic changes in target entities.

### RELATED APPLICATIONS

This application claims priority to U.S. Provisional application, U.S. Ser. No. 61/960,143, filed on Sep. 11, 2013, the content of which is hereby incorporated by reference in its entirety.

### FIELD OF THE INVENTION

The present invention relates generally to screening of populations of organisms or biomaterials isolated therefrom, and more specifically to the identification of biomolecules, bioactive molecules and bioactivities through high throughput screening techniques, including fluorescence activated cell sorting (FACS).

### BACKGROUND

Identification of biomolecules such as antibodies and other proteins that interact with mammalian cell surface-associated entities has been complicated by the inability to reproducibly present such cell surface-associated entities to populations of biomolecules.

Cell membrane embedded proteins such as ion channels, enzyme-linked receptors, and G protein-coupled receptors (GPCRs) represent a substantial class of therapeutic target, with enzyme-linked receptor binding antibodies, ion channel-directed, and GPCR-directed small molecule drugs utilized in a wide range of therapeutic indications; these receptors are the primary receivers of communication by a cell from its extracellular environment and they are important mediators of cell to cell communication.

Cell surface receptor structure is divided into extracellular domains, transmembrane domains, and intracellular domains. To date, successful targeting of transmembrane domains and intracellular domains has been substantially limited to small molecule compounds, and even screening for binding to a cell surface receptor's extracellular domain is problematic depending upon the method of producing and presenting the extracellular domain.

There has been difficulty in identifying and validating proteins interacting with mammalian cell surface receptors and other membrane associated proteins; as it has been difficult to present the surface-localized protein in a native context for discovery. Frequently, making such proteins recombinantly, such as in a prokaryotic host disrupts the native environment of the protein, which is important for proper folding. Additionally, recombinant protein production often necessitates a truncation of the protein, thereby removing putatively valuable epitopes from presentation. Prior efforts involving inoculating cells overexpressing the mammalian cell surface protein into mice or rabbits in order to produce antibody hybridomas frequently results in production of antibodies that are directed largely to unrelated proteins that are presented on the mammalian cell surface. Panning phage or yeast against cells recombinantly expressing a protein of interest suffers from non-specific binding of the yeast or phage, and inaccessibility of the putative yeast- or phage-displayed antibody to the protein of interest due to steric inhibition by the yeast or phage.

Thus, there is a critical need for methods and compositions to screen biomolecule binders to proteins and other entities present on the cell surface.

### SUMMARY OF THE INVENTION

Aspects of the invention provide solutions to the deficiencies encountered in current high-throughput screening assays for molecules that interact with cell surface proteins. Certain aspects of the invention relate to the use of gel microdrops that comprise a limited permeability material, such as a hydrogel, to encase and hold in place a target entity, such as a vertebrate or mammalian cell, having a ligand of interest on its surface (a target moiety) and a secretory entity, such as a yeast cell, that produces a binder to the ligand of interest (a targeting moiety), where the binder (targeting moiety) is freely diffusible within the gel microdrop between the secretory entity and the target entity. Gel microdrops comprising a limited permeability material, a secretory entity and a target entity that is a mammalian cell are also referred to herein as “mammalian cell complexes.” Advantageously, the target entity can display the desired cell surface-associated entities (the target moieties), such as, e.g. ion channels, enzyme-linked receptors, and G protein-coupled receptors (GPCRs) in a native context and large numbers of secretory entities may be rapidly screened for interactions of the targeting moiety to the target moiety and easily selected when a significant interaction is detected. The methods and compositions described herein are suitable for high throughput screens, for example, they are adaptable to microfluidic setups and automated cell sorting, such as fluorescent-activated cell sorting (FACS), thus making the screening of these interactions, the identification and validation of new therapeutic entities faster, easier and more efficacious.

Aspects of the invention relate to gel microdrop compositions that comprise a limited permeability material, a secretory entity that secretes a targeting moiety into the limited permeability material, and a first target entity comprising a target moiety, with the proviso that the gel microdrop does not contain a second target entity that is distinct from the first target entity. The target entity and the secretory entity both are suspended in the limited permeability material and the limited permeability material is substantially impermeable for both the target entity and the secretory entity. The limited permeability material is, however, permeable for the secreted targeting moiety.

In certain embodiments, the microdrop is substantially spherical and can have a diameter of from about 10 microns to about 100 microns. The microdrop can have a volume of from about 4 picoliters to about 4 nanoliters and may be suspended in a medium, buffer, oil phase, or emulsion. The microdrop may be generated by a microfluidics-based method.

In certain embodiments, the limited permeability material comprises a polymer matrix, such as a hydrogel. The hydrogel may comprise agarose, carrageenan, alginate, alginate-polylysine, collagen, cellulose, methylcellulose, gelatin, chitosan, extracellular matrix, dextran, starch, inulin, heparin, hyaluronan, fibrin, polyvinyl alcohol, poly(N-vinyl-2-pyrrolidone), polyethylene glycol, poly(hydroxyethyl methacrylate), acrylate polymers and sodium polyacrylate, polydimethyl siloxane, cis-polyisoprene, Puramatrix™, poly-divenylbenzene, polyurethane, or polyacrylamide. The polymer matrix of the limited permeability material can have a porosity of from about 10 nm to 5 microns.

In certain embodiments, the secretory entity is a cellular entity, such as a yeast cell, a bacterial cell, or a B cell.

In other embodiments, the secretory entity is a non-cellular entity such as a ribosome-mRNA complex.

In some embodiments, the non-cellular entity comprises cleavable targeting moieties supported on a solid surface, such as a bead, that are secreted upon cleavage from the solid surface.

In certain embodiments, the targeting moiety is a polypeptide such as an antibody or an antibody-like polypeptide.

In some embodiments, the secreted targeting moiety specifically binds to the target moiety of the target entity and is retained in the microdrop.

In other embodiments, the secreted targeting moiety does not specifically bind to the target moiety of the target entity and is capable of diffusing out of the limited permeability material of the microdrop.

In some embodiments, the target entity is a cellular entity, such as a mammalian cell, a vertebrate cell or an invertebrate cell. If the target entity is a mammalian cell, the cell may be a human cell that is optionally healthy or normal, and optionally neoplastic or atypical. The target entity can be a cell line.

In other embodiments, the target entity is a non-cellular entity. The target entity may comprise target moieties supported on a solid surface, such as a bead.

In certain embodiments, the target moiety is an antigen. The antigen can be a cell membrane-associated polypeptide such as an ion channel protein, a transporter protein, or a G protein coupled receptor (GPCR), including C3aR, C5aR, FPRL, CXCR4, CCR4, CCR5, CCR2, CCR9, CCR8, GCG-R, GLP-1R, VPAC-1, LGR5, CRTH2, CXCR3, MLNR, ADRA2C, OPRL1, DRD2, HCRTR1, HCRTR2, EDNRA, P2RY12, PTGER4, LTBR4, OXTR, PTGFR, NPY2R, CXCR2, MTNR1B, TACR2, CX3CR1, HTR1F, HTR6, NPSR, SSTR4, SSTR5, SQPR2, PTGER2, SSTR2, CHRM2, CHRM4, ADRB1, ADRB2, SSTR3, GiPR, PTH1R, S1P3, CRTH2, CXCR1, CXCR6, GLP1R, LPAR2, P2RY2, and VIPR1. The cell-membrane associated polypeptide can be a full-length form. The cell-membrane associated polypeptide may, in some embodiments, not be an antigenic fragment of a full length protein.

In certain embodiments, the microdrops of the compositions described herein contain secretory entities and target entities in a ratio of from about 10:1 to about 1:5, in a ratio of about 1:1, in a ratio of about 2:1, in a ratio of about 5:1, or in a ratio of about 10:1.

In some embodiments, the microdrops contain a single secretory entity and a single target entity.

Aspects of the invention relate to libraries of targeting moieties comprising a plurality of microdrops as described herein, wherein the plurality of microdrops comprises a plurality of distinct targeting moieties secreted by a plurality of secretory entities.

In certain embodiments, the plurality of microdrops comprises a single target entity comprising a single target moiety.

In certain embodiments, the libraries comprise a plurality of secretory entities that are a library of yeast expressing and secreting a plurality of targeting moieties such as antibody polypeptides or antibody-like polypeptides. The libraries may comprise from about 10³ clones to about 10¹⁰ clones, or from about 10⁶ clones to about 10⁹ clones. The target entity used in the libraries can be a mammalian cell. Optionally, the target moiety antigen is a cell membrane-associated polypeptide.

Aspects of the invention relate to methods for detecting a targeting moiety with affinity to a target moiety. The methods comprise the steps of a) making or providing a gel microdrop composition as described herein, b) removing a targeting moiety not bound to a target moiety, c) contacting the microdrop with a detection entity comprising a detectable moiety, wherein the detection moiety is capable of binding to the targeting moiety, d) removing a detection moiety not bound to a targeting moiety, and e) detecting the detectable moiety, wherein if the detectable moiety is detected, the targeting moiety has affinity to the target moiety.

Further provided are methods for isolating a targeting moiety with affinity to a target moiety. The methods comprise the steps of a) making or providing a gel microdrop composition as described herein, b) removing a targeting moiety not bound to a target moiety, c) contacting the microdrop with a detection entity comprising a detectable moiety, wherein the detection moiety is capable of binding to the targeting moiety, d) removing a detection moiety not bound to a targeting moiety, e) selecting a microdrop for which the detectable moiety is detected, wherein if the detectable moiety is detected, the targeting moiety has affinity to the target moiety, f) collecting the selected microdrop, and g) isolating the secretory entity that secretes the targeting moiety with affinity to the target moiety.

For the methods described herein, isolating the secretory entity may include dissolution of the limited permeability material, for example through de-polymerization. A detection moiety suitable for the methods can be an antibody specific for the targeting moiety. The detectable moiety can be a fluorescent molecule allowing selection of the microdrop using fluorescent activated cell sorting (FACS).

Further provided are methods of making a gel microdrop composition. The methods comprise the steps of a) combining: i) a monomer capable of forming a limited permeability material upon polymerization, ii) a secretory entity capable of secreting a targeting moiety, and iii) a target entity comprising a target moiety, b) forming droplets of the combination of step (a), and c) polymerizing the monomers of the droplets formed in step (b) to produce gel microdrops comprising a limited permeability material.

In certain embodiments, the polymerization is induced by a temperature change of the ambient temperature of the microdrop.

In other embodiments, the polymerization is induced by contacting the microdrop with an enzyme capable of polymerizing the monomers.

In yet other embodiments, the polymerization is induced by contacting the microdrop with a chemical polymerization agent capable of polymerizing the monomers.

For any method of making a gel microdrop, the droplets may be formed using a microfuidic apparatus.

Further provided are methods of making a library of targeting moieties comprising a plurality of microdrops described herein. The methods comprise the steps of a) combining: i) a monomer capable of forming a limited permeability material upon polymerization, ii) a plurality of secretory entities capable of secreting a targeting moiety, wherein the secretory entities are distinct from one another, and iii) a plurality of target entities comprising a target moiety, wherein the target entities are substantially the same, b) forming droplets of the combination of step (a), wherein the majority of formed droplets comprises secretory entities and target entities in a ratio of from about 10:1 to about 1:1, and c) polymerizing the monomers of the droplets formed in step (b) to produce gel microdrops comprising a limited permeability material.

In certain embodiments, the polymerization is induced by a temperature change of the ambient temperature of the microdrop.

In other embodiments, the polymerization is induced by contacting the microdrop with an enzyme capable of polymerizing the monomers.

In yet other embodiments, the polymerization is induced by contacting the microdrop with a chemical polymerization agent capable of polymerizing the monomers.

In still other embodiments, the polymerization is induced by contacting the microdrop with photons of light.

For any method of making libraries of microdrops, the droplets may be formed using a microfuidic apparatus.

Further provided are methods for isolating a targeting moiety with high affinity to a target moiety from a library of targeting moieties. The methods comprise the steps of a) making or providing a library of targeting moieties comprising a plurality of microdrops as described herein, b) removing a targeting moiety not bound to a target moiety, c) contacting the microdrop with a detection entity comprising a detectable moiety, wherein the detection moiety is capable of binding to the targeting moiety, d) removing a detection moiety not bound to a targeting moiety, e) selecting a microdrop for which the detectable moiety is detected, wherein if the detectable moiety is detected, the targeting moiety has affinity to the target moiety, f) collecting the selected microdrop, g) isolating the secretory entity that secretes the targeting moiety with affinity to the target moiety, and h) repeating steps (a) to (g) with the isolated secretory entity from step (g), and progressively selecting the microdrops with the highest signal for the detectable moiety in (e), thereby isolating a targeting moiety with high affinity to a target moiety from a library of targeting moieties.

For the methods described herein, isolating the secretory entity may include dissolution of the limited permeability material, for example through de-polymerization. A detection moiety suitable for the methods can be an antibody specific for the targeting moiety. The detectable moiety can be a fluorescent molecule allowing selection of the microdrop using fluorescent activated cell sorting (FACS).

In certain embodiments, the majority of the plurality of microdrops comprises secretory entities and target entities in a ratio of from about 10:1 to about 1:1.

Further provided are methods for identifying a targeting moiety from a library of targeting moieties. The methods comprise the steps of a) making or providing a library of targeting moieties comprising a plurality of microdrops as described herein, b) removing a targeting moiety not bound to a target moiety, c) contacting the microdrop with a first and a second detection entity comprising a detectable moiety, wherein the first detection entity is capable of binding to the targeting moiety, and the second detection entity is capable of binding to the target entity upon a phenotypic change in the target entity, d) removing a first detection entity not bound to a targeting moiety, and removing a second detection entity not bound to a target entity, and e) selecting a microdrop for which the detectable moiety of the first and the second detection entity is detected, wherein if the first detectable moiety is detected, the targeting moiety has affinity to the target moiety, and if the second detectable moiety is detected, the targeting moiety induces a phenotypic change in the target entity. The method may further comprise the steps off) collecting the selected microdrop, and g) isolating the secretory entity that secretes the targeting moiety.

In certain embodiments, the phenotypic change in the target entity induced by the targeting moiety is apoptosis, a change in the proteome, a change in the metabolome, a change in the epigenome, or a change in the transcriptome. Where the phenotypic change is apoptosis the second detection entity can be DAPI stain, ethidium bromide stain or propidium iodide stain.

Aspects of the invention relate to mammalian cell complexes comprising a mammalian cell, a secretory entity and a limited permeability material. The mammalian cell and the secretory entity are present in and not substantially capable of permeating through the limited permeability material and the secretory entity is capable of secreting a targeting polypeptide.

In certain embodiments, the mammalian cell may comprise a target antigen and the targeting polypeptide comprises an antibody or antibody-like polypeptide.

In certain embodiments, the antibody or antibody-like polypeptide are capable of permeating through the limited permeability material.

In some embodiments, the antibody or antibody-like polypeptide are capable of specifically binding the target antigen.

In other embodiments, the antibody or antibody-like polypeptide are capable of specifically binding an antigen other than the target antigen.

In certain embodiments, the antibody or antibody-like polypeptide comprise a detectable moiety, such as a fluorescent moiety.

In some embodiment, the mammalian complexes further comprise a detection agent, wherein the detection agent comprises a detectable moiety, and wherein the detection agent is capable of specifically binding to the antibody or antibody-like polypeptide.

In other embodiments, the antibody or antibody-like polypeptide comprises a detection tag, and the detection agent is capable of binding to the detection tag.

In certain embodiments, the antibody or antibody-like polypeptide comprise a separation moiety such as a moiety that is magnetic or capable being bound by a magnet.

In some embodiments, the mammalian complexes further comprise a separation agent, wherein the separation agent comprises a separation moiety, and wherein the separation agent is capable of specifically binding to the antibody or antibody-like polypeptide.

In some embodiments, the separation agent comprises an antibody or antibody-like polypeptide and wherein the separation moiety comprises a magnetic particle.

In some embodiments, the antibody or antibody-like polypeptide comprise a secretion leader peptide optionally encoded by a nucleic acid sequence.

In some embodiments, the mammalian cell comprises a cell surface receptor and the targeting polypeptide comprises a ligand capable of specifically binding to the cell surface receptor.

In specific embodiments, the mammalian cell comprises an antigen and the targeting polypeptide comprises an antibody or antibody-like polypeptide.

In other specific embodiments, the mammalian cell comprises a substrate and the targeting polypeptide comprises an enzyme capable of acting upon the substrate.

In yet other specific embodiments, the mammalian cell comprises an enzyme and the targeting polypeptide comprises a substrate capable of being acted upon by the enzyme.

In yet other specific embodiments, the targeting polypeptide comprises a cell penetrating polypeptide, and the mammalian cell is detectably modified upon penetration by the cell penetrating polypeptide.

In certain embodiments, mammalian complexes are provided, wherein the mammalian cell and the secretory entity are present in the limited permeability material at a ratio from about 5:1, at a ratio from about 1:1, or at a ratio from about 1:10.

In some embodiments, the secretory entity comprises a bacterial cell, a yeast cell or a ribosome-mRNA complex.

In other embodiments, the secretory entity comprises a plant cell or a mammalian cell

In certain embodiments, mammalian complexes are provided, wherein the limited permeability material has a porosity of from about 10 nm to about 1000 nm. The limited permeability material can be substantially spherical and has a diameter less than about 100 microns.

In certain embodiments, mammalian complexes are provided, wherein all secretory entities present in the limited permeability material are capable of secreting the same targeting polypeptide. The targeting polypeptide may be capable of binding to the mammalian cell, and such binding may cause the transduction of a cell signal.

Aspects of the invention relate to a library comprising a plurality of mammalian cell complexes described herein. The library may further comprise a retention device capable of individually retaining the cell complexes present in the library. The retention device may comprise a solid or semi-solid support material. Alternatively, the retention device comprises a liquid or gel support material.

Aspects of the invention relate to methods of displaying a secreted engineered protein complex on a mammalian cell. The methods comprise the steps of a) providing a mammalian cell complex comprising a mammalian cell, a secretory entity and a limited permeability material, wherein the mammalian cell and the secretory entity are present in and not substantially capable of permeating through the limited permeability material, wherein the secretory entity comprises a first nucleic acid, and b) incubating the mammalian cell complex under conditions sufficient for expressing by the secretory entity a engineered protein encoded by the first nucleic acid, wherein the engineered protein is secreted by the secretory entity, and wherein the engineered protein binds to a binding moiety present on the mammalian cell, thereby forming a secreted engineered protein complex on the mammalian cell.

Further provided are methods of selecting a mammalian cell. The methods comprise the steps of a) providing a mammalian cell complex comprising a mammalian cell, a secretory entity and a limited permeability material, wherein the mammalian cell expresses a target polypeptide, wherein the target polypeptide is located at the plasma membrane of the mammalian cell, wherein the mammalian cell and the secretory entity are present in and not substantially capable of permeating through the limited permeability material, wherein the secretory entity comprises a first nucleic acid, b) incubating the mammalian cell complex under conditions sufficient for expressing by the secretory entity a engineered protein encoded by the first nucleic acid, wherein the engineered protein is secreted by the secretory entity, and wherein the engineered protein binds to the target polypeptide on the mammalian cell, and c) detecting the engineered protein bound to the target polypeptide, thereby selecting the mammalian cell.

Further provided are methods of screening an engineered protein. The methods comprise the steps of a) providing a plurality of mammalian cell complexes, wherein each mammalian cell complex independently comprises a mammalian cell, a secretory entity and a limited permeability material, wherein the mammalian cell expresses a target polypeptide, wherein the target polypeptide is located at the plasma membrane of the mammalian cell, wherein the mammalian cell and the secretory entity are present in and not substantially capable of permeating, through the limited permeability material, wherein the secretory entity comprises a first nucleic acid, b) incubating the plurality of mammalian cell complexes under conditions sufficient for expressing by the secretory entity a engineered protein encoded by the first nucleic acid, wherein the engineered protein is secreted by the secretory entity, and wherein in at least one mammalian cell complex the engineered protein binds to the target polypeptide on the mammalian cell, c) detecting the engineered protein bound to the target polypeptide, and d) identifying the detected engineered protein, thereby screening the engineered protein.

In certain embodiments, the methods comprise providing at least about 1×10⁴ mammalian cell complexes. For some methods, each engineered protein comprises an antibody, and at least about 1×10⁵ unique engineered proteins are present in the plurality of mammalian cell complexes.

Further provided are methods of sorting mammalian cells. The methods comprise the steps of providing a first mammalian cell complex comprising a mammalian cell, a secretory entity and a limited permeability material, wherein the mammalian cell expresses a target polypeptide, wherein the target polypeptide is located at the plasma membrane of the mammalian cell, wherein the mammalian cell and the secretory entity are present in and not substantially capable of permeating through the limited permeability material, wherein the secretory entity comprises a first nucleic acid, b) incubating the mammalian cell complex under conditions sufficient for expressing by the secretory entity a engineered protein encoded by the first nucleic acid, wherein the engineered protein is secreted by the secretory entity, and wherein the engineered protein binds to the target polypeptide on the mammalian cell, and c) detecting the engineered protein bound to the target polypeptide, thereby selecting the mammalian cell.

Aspects of the invention relate to gel microdrop compositions comprising a limited permeability material, a first secretory entity that secretes a targeting moiety into the limited permeability material, and a second secretory entity that secretes a target moiety into the limited permeability material. The first and the second secretory entity are not the same and both are suspended in the limited permeability material. The limited permeability material is substantially impermeable for both secretory entities. The limited permeability material is permeable for both the secreted targeting moiety and the secreted target moiety, but substantially impermeable for a binding complex comprising the targeting moiety and the target moiety.

In certain embodiments, the first and the second secretory entities are cellular entities.

In some embodiments, the first secretory entity secretes an antigen and the second secretory entity secretes an antibody.

In other embodiments, the first secretory entity secretes a receptor molecule and the second secretory entity secretes a ligand.

In yet other embodiments, the first secretory entity secretes an enzyme and the second secretory entity secretes a substrate.

In yet another embodiment, the first secretory entity secretes an apoenzyme and the second secretory entity secretes a cofactor.

Aspects of the invention relate to gel microdrop compositions comprising a limited permeability material, a first binding entity comprising a targeting moiety, and a second binding entity comprising a target moiety. The first and the second binding entity are not the same and both are suspended in the limited permeability material. The limited permeability material is substantially impermeable for both binding entities, and binding of the targeting moiety of the first binding entity to the target moiety of the second binding entity causes a phenotypic change in one or both of the binding entities.

In some embodiments, the first and second binding entities are cellular.

In other embodiments, the first or the second binding entity is cellular and the other entity is non-cellular.

In certain embodiments, the phenotypic change is apoptosis, or a change in the proteome, the metabolome, the epigenome, or the transcriptome.

Aspects of the invention relate to gel microdrop compositions comprising a limited permeability material, a target entity comprising a detectable moiety, and a capture entity capable of engulfing the target entity. The target entity and the capture entity both are suspended in the limited permeability material, and the limited permeability material is substantially impermeable for the capture entity.

In some embodiments the limited permeability material is permeable for the target entity, while in alternative embodiments, the limited permeability material is substantially impermeable for the target entity.

In some embodiments, the target entity is a non-cellular entity, such as a bead, while in alternative embodiments, the target entity is a cellular entity.

In certain embodiments, engulfment of the target entity by the capture entity changes a detectable characteristic of the detectable moiety, such as a detectable change in the wavelength of light emitted from the detectable moiety when it is excited.

In some embodiments, the capture entity is a cellular entity such as a macrophage.

Aspects of the invention relate to methods for producing a targeting moiety with high affinity to a target moiety from a library of targeting moieties. The methods comprise the steps of a) making or providing a library of targeting moieties comprising a plurality of microdrops described herein, b) removing a targeting moiety not bound to a target moiety, c) contacting the microdrop with a detection entity comprising a detectable moiety, wherein the detection moiety is capable of binding to the targeting moiety, d) removing a detection moiety not bound to a targeting moiety, e) selecting a microdrop for which the detectable moiety is detected, wherein if the detectable moiety is detected, the targeting moiety has affinity to the target moiety, f) collecting the selected microdrop, g) isolating the secretory entity that secretes the targeting moiety with affinity to the target moiety, and h) repeating steps (a) to (g) with the isolated secretory entity from step (g), and progressively selecting the microdrops with the highest signal for the detectable moiety in (e), wherein upon repetition a targeting moiety with high affinity to a target moiety is identified from the library of targeting moieties, i) isolating the secretory entity that secretes the high affinity targeting moiety identified in step (h), j) propagating the isolated secretory entity from step (i), and k) isolating the high affinity targeting moiety.

Aspects of the invention relate to methods for producing a targeting moiety from a library of targeting moieties. The methods comprise the steps of a) making or providing a library of targeting moieties comprising a plurality of microdrops described herein, b) removing a targeting moiety not bound to a target moiety, c) contacting the microdrop with a first and a second detection entity comprising a detectable moiety, wherein the first detection entity is capable of binding to the targeting moiety, and the second detection entity is capable of binding to the target entity upon a phenotypic change in the target entity, d) removing a first detection entity not bound to a targeting moiety, and removing a second detection entity not bound to a target entity, e) selecting a microdrop for which the detectable moiety of the first and the second detection entity is detected, wherein if the first detectable moiety is detected, the targeting moiety has affinity to the target moiety, and if the second detectable moiety is detected, the targeting moiety induces a phenotypic change in the target entity, f) collecting the selected microdrop, g) isolating the secretory entity that secretes the targeting moiety with affinity to the target moiety, and h) repeating steps (a) to (f) with the isolated secretory entity from step (g), and progressively selecting the microdrops with the highest signal for the detectable moiety in (e), wherein upon repetition a targeting moiety with high affinity to a target moiety is identified from the library of targeting moieties, i) isolating the secretory entity that secretes the high affinity targeting moiety identified in step (h), j) propagating the isolated secretory entity from step (i), and k) isolating the high affinity targeting moiety.

In certain embodiments, the methods further comprise preserving the high affinity targeting moiety, for example by dissolving the targeting moiety in a medium comprising a preservative or drying the targeting moiety, such as freeze-drying.

### BRIEF DESCRIPTION OF THE FIGURES

FIG. 1 is a flow chart describing a method of high-throughput screening of targeting entities using the microdrop compositions described herein in accordance with an example of the invention;

FIG. 2A is a schematic of the generation of a microdrop that contains ErbB2-coated beads and HERCEPTIN-secreting yeast in accordance with an example of the invention;

FIG. 2B is an image of ErbB2-coated beads and HERCEPTIN-secreting yeast in agarose droplets taken under a fluorescence microscope;

FIG. 3A is a schematic (left panel) of a microdrop containing HERCEPTIN-secreting yeast and BSA-coated beads (negative control) and a corresponding FACS histogram (right panel) of the labeled HERCEPTIN signal of the droplets;

FIG. 3B is a schematic (left panel) of a microdrop containing ErbB2-coated beads and non-secreting yeast (negative control) and a corresponding FACS histogram (right panel) of the labeled HERCEPTIN signal, with FIG. 3D showing a FACS plot with the position of the sort gate for HERCEPTIN-positive droplets;

FIG. 3C is a schematic (left panel) of a microdrop containing ErbB2-coated beads and HERCEPTIN-secreting yeast and a corresponding FACS histogram (right panel) of the labeled HERCEPTIN signal, with FIG. 3F showing a FACS plot with the position of the sort gate for HERCEPTIN-positive droplets;

FIG. 4A is a FACS histogram showing the distribution of a mixture containing 5% Herceptin-secreting and 95% non-secreting yeast cells;

FIG. 4B is a FACS plot with the position of the sort gate for HERCEPTIN-positive droplets;

FIG. 4C is a photograph of an agar plate on which yeast isolated from the HERCEPTIN-positive droplets after droplet sorting are cultured;

FIG. 4D is a bar chart showing enrichment of the sorted droplet population in HERCEPTIN-secreting yeast;

FIG. 5 is an image of viable HEK293 cells encapsulated in agarose microdroplets taken under a fluorescence microscope;

FIG. 6 is a flow chart describing an embodiment in which a phenotypic change is measured upon binding of the targeting moiety to a target entity using the microdrop compositions described herein in accordance with an example of the invention;

FIG. 7 is a flow chart describing an embodiment in which a third entity is included in the microdrop compositions described herein in accordance with an example of the invention.

### DETAILED DESCRIPTION OF THE INVENTION

This invention relates in part to methods and compositions for the identification, characterization and maturation of targeting moieties, such as binding polypeptides that functionally interact with proteins, carbohydrates, lipids or other biological target moieties displayed by a target entity (e.g. target moieties that are present on the surface of a mammalian cell or on a mammalian cell membrane), while retaining the linkage between genotypic content of the producer of the targeting entity (such as a secretory entity) and the detectable binding activity of the targeting moiety to the target moiety. In some embodiments, targeting moieties (e.g. binding polypeptides such as antibodies) are capable of altering the function of the bound target moiety (e.g. cell surface-associated moiety) or the phenotypic characteristics of the target entity (e.g. a cell expressing the target moiety). In this way high affinity targeting moieties can be identified and isolated that also have physiological effects on their target entities, such as changes in the viability or growth of a target cell.

Encapsulation methods using microdrops or capsules to screen for secreted molecules, secreted effector molecules, and ligand binding proteins have been proposed in the art, e.g. U.S. Pat. No. 6,806,058 “SECRETIONS OF PROTEINS BY ENCAPSULATION” and U.S. Pat. No. 8,030,095 “GEL MICRODROP COMPOSITION AND METHOD OF USING THE SAME” and U.S. Publ. No. 2004/0241759 “HIGH THROUGHPUT SCREENING OF LIBRARIES.” These methods have in common that they attempt to maintain the secreted molecules of interest, emitted from secretory entities, in the encapsulated space to screen and analyze the secreted molecules. The methods further have in common that they employ very complex microdrop compositions. Some methods employ complicated set ups in which the matrix or encapsulation materials that make up the microdrop or capsule comprise various capturing moieties capable of binding the secreted molecules in order to retain them. This requires specific conjugating chemistries and limits the choice of encapsulating materials. They also require multilayered antibody or ligand interactions to determine if binding has occurred. Another approach requires a plurality of reporter particles that can capture the secreted effector molecule and relies on changes of optical signals between the reporter molecules upon binding of the effector molecule to detect binding. Such methods are not adaptable to high-throughput screens. For example, the changes in the relative signals can best be detected microscopically, and high-throughput cell sorting methods such as fluorescent-activated cell sorting (FACS) do not easily offer the capability to sort according to these relative changes of detectable signals. Further, high-throughput screens require the ability to create vast libraries of microdrops with a consistent distribution of secretory entities and reporter particles within each droplet. Microdrops that require three different entities to come together in specific stoichiometries (relative quantities) are very difficult to produce, either by batch approaches or through the use of microfluidic devices. In a random distribution, some microdrops will contain no entities, some will contain the secretory entity, some will contain one or the other reporter particle, some will contain the two different reporter particles but no secretory entity, and some will contain all three entities in one droplet. Controlling the presence of entities in the microdrops becomes even more difficult if two of the entities are preferably in the same abundance in the microdrop but both are in higher abundance than the third entity. Only under the most optimal circumstances will any significant number of functional microdroplets form (i.e. those that have all three entities with the correct stoichiometry).

Surprisingly, it has now been found that a simple microdrop set up can be effectively employed to allow large-scale or high-throughput screening of interactions of secreted targeting moieties and target entities, fast and efficient selection of targeting moieties that show affinity to the target entities, and high-yield recovery of secretory entities that produce the targeting moieties that allows for rapid isolation, characterization and/or production of the identified targeting moieties.

### DEFINITIONS

A “binding entity” generally is a cellular entity such as a prokaryotic or eukaryotic cell that exhibits, usually on its surface one or more target moieties or alternatively targeting moieties that are capable of interacting with, e.g. specific binding of, another binding entity that may or may not be distinct. Cellular binding entities include mammalian cells, vertebrate cells, and invertebrate cells, yeast and prokaryotes, such as bacteria. Binding entities also include non-cellular entities, e.g. binding entities that display target moieties or targeting moieties on a solid surface, such as a bead. In certain embodiments, a first binding entity comprises a targeting moiety and a second binding entity comprises a target moiety and the first and the second binding entity are not the same, i.e. distinct, e.g. they are different types of cell (e.g. a vertebrate cell and a yeast cell, or a mammalian cell and a bacterium, etc.) or they are a cellular binding entity and a non-cellular binding entity.

A “capture entity” generally is a cellular entity such as a prokaryotic or eukaryotic cell that is capable of engulfing a target entity. “Capable of engulfing a target entity” as used herein means that the capture entity interacts with and incorporates the target entity, e.g. by phagocytosis, receptor-mediated endocytosis, or pinocytosis. In some embodiments, passive influx through the membrane or ion channel mediated influx of the target entity are also included in the meaning of “engulfing a target entity.” Capture entities include mammalian cells, vertebrate cells, and invertebrate cells, yeast and prokaryotes, such as bacteria. In some embodiments, the capture entity is a macrophage. Capture entities may engulf other cellular entities, e.g. mammalian cells or bacteria, or non-cellular entities, such as, e.g. beads. For example, beads may be engulfed that comprise detection entities.

“Cell membrane associated polypeptides,” as used herein include ion channel proteins, transporter proteins, and G protein coupled receptors (GPCR), as well as subunits and functional fragments thereof. In some embodiments, the proteins or subunits are full length and are not functional fragments.

“G protein coupled receptors (GPCR)” include 5-Hydroxytryptamine receptors, Acetylcholine receptors (muscarinic), Adenosine receptors, Adrenoceptors, Angiotensin receptors, Apelin receptor, Bile acid receptor, Bombesin receptors, Bradykinin receptors, Cannabinoid receptors, Chemerin receptor, Chemokine receptors, Cholecystokinin receptors, Complement peptide receptors, Dopamine receptors, Endothelin receptors, Estrogen (G protein-coupled) receptor, Formylpeptide receptors, Free fatty acid receptors, Galanin receptors, Ghrelin receptor, Glycoprotein hormone receptors, Gonadotrophin-releasing hormone receptors, Histamine receptors, Hydroxycarboxylic acid receptors, Kisspeptin receptor, Leukotriene receptors, Lysophospholipid (LPA) receptors, Lysophospholipid (S1P) receptors, Melanin-concentrating hormone receptors, Melanocortin receptors, Melatonin receptors, Motilin receptor, Neuromedin U receptors, Neuropeptide FF/neuropeptide AF receptors, Neuropeptide S receptor, Neuropeptide W/neuropeptide B receptors, Neuropeptide Y receptors, Neurotensin receptors, Opioid receptors, Orexin receptors, Oxoglutarate receptor, P2Y receptors, Peptide P518 receptor, Platelet-activating factor receptor, Prokineticin receptors, Prolactin-releasing peptide receptor, Prostanoid receptors, Proteinase-activated receptors, Relaxin family peptide receptors, Somatostatin receptors, Succinate receptor, Tachykinin receptors, Thyrotropin-releasing hormone receptors, Trace amine receptor, Urotensin receptor, Vasopressin and oxytocin receptors, and Class A Orphans.

“Ion channels” include Voltage-gated ion channels, CatSper and Two-Pore channels, Cyclic nucleotide-regulated channels, Potassium channels, Calcium-activated potassium channels, Inwardly rectifying potassium channels, Two-P potassium channels, Voltage-gated potassium channels, Transient Receptor Potential channels, Voltage-gated calcium channels, Voltage-gated sodium channels, Ligand-gated ion channels, 5-HT3 receptors, GABAA receptors, Glycine receptors, Ionotropic glutamate receptors, Nicotinic acetylcholine receptors, P2X receptors, and Zink-activated ion channel (ZAC).

“Transporters” include pores and channels, such as alpha-helical channels, and beta-strand porins; electrochemical-potential-driven transporters, such as, uniporters, symporters and antiporters; primary active transporters, such as P—P-bond-hydrolysis-driven transporters (e.g. ATP-binding-cassette superfamily, ABC-type exporters), decarboxylation-driven transporters (e.g. Na⁺-transporting carboxylic acid decarboxylase), methyl-transfer-driven transporters (e.g. Na+-transporting methyltetrahydromethanopterin-CoM methyltransferase), oxidoreduction-driven transporters (e.g. proton (H⁺ or Na⁺)-translocating NADH dehydrogenases), light-driven transporters; phosphotransferases; and transmembrane electron carriers.

The term “construct” refers to a recombinant nucleic acid sequence, generally recombinant DNA, that has been generated for the purpose of the expression of a specific nucleotide sequence(s), or is to be used in the construction of other recombinant nucleotide sequences. A construct might be present in a vector or in a genome. The term “recombinant” refers to a polynucleotide or polypeptide that does not naturally occur in a host cell, or a cell or organism containing a recombinant polynucleotide or polypeptide. The term “selective marker” refers to a protein capable of expression in a host that allows for ease of selection of those hosts containing an introduced nucleic acid or vector. Examples of selectable markers include, but are not limited to, proteins that confer resistance to antimicrobial agents (e.g., hygromycin, bleomycin, or chloramphenicol), proteins that confer a metabolic advantage, such as a nutritional advantage on the host cell, as well as proteins that confer a functional or phenotypic advantage (e.g., cell division) on a cell. The term “expression”, as used herein, refers to the process by which a polypeptide is produced based on the nucleic acid sequence of a gene. The process includes both transcription and translation. The term “introduced” in the context of inserting a nucleic acid sequence into a cell, means “transfection”, or “transformation” or “transduction” and includes reference to the incorporation of a nucleic acid sequence (e.g. DNA or RNA) into a eukaryotic or prokaryotic cell wherein the nucleic acid sequence may be incorporated into the genome of the cell (e.g., chromosome, plasmid, plastid, or mitochondrial DNA), converted into an autonomous replicon, or transiently expressed (e.g., transfected mRNA). The term “coding sequence” refers to a nucleic acid sequence that once transcribed and translated produces a protein, for example, in vivo, when placed under the control of appropriate regulatory elements. A coding sequence as used herein may have a continuous ORF or might have an ORF interrupted by the presence of introns or non-coding sequences. In this embodiment, the non-coding sequences are spliced out from the pre-mRNA to produce a mature mRNA.

The term “contacting” means to bring or put together. As such, a first item is contacted with a second item when the two items are brought or put together, e.g., by touching them to each other or combining them in the same solution. For example, a target moiety (e.g. an antigen) and a target moiety (e.g. an antigen-specific antibody) are put together in the same solution of defined space to bring about binding of the targeting moiety (antibody) to the target moiety (antigen). Similarly, a detection entity (e.g. a fluorescently labeled antibody) and a targeting moiety (e.g. a target moiety-specific antibody) are put together in the same solution or defined space to bring about binding of the detection entity to the targeting moiety.

A “detection entity,” as used herein is an entity that is capable of specifically recognizing another entity (e.g. a target moiety, a targeting moiety, a target entity, or a secretory entity) and that comprises a detectable moiety (such as a fluorescent moiety), thereby facilitating detection of the other entity. Typically, the detection entity is an antibody labeled with a detectable (e.g. fluorescent) moiety. Particularly suitable are antibodies that specifically recognize an invariant part of the targeting moiety so that selective binding of the antigen-specific part of the targeting moiety to a target moiety can be visualized, such as a fluorophore-labeled anti-IgG antibody.

A “detectable moiety” refers to an entity that produces electromagnetic radiation (including infrared radiation, visible light, ultraviolet radiation, X-rays and gamma rays) that can be detected by a photodetector, such as a fluorescence-activated cell sorter (FACS machine), a light microscope, a spectrophotometer, a fluorescent microscope, a fluorescent sample reader, a 3D tomographer, or a camera. The term “fluorescent” molecule refers to an entity that produces a signal (the emission of light) after it has absorbed light or other electromagnetic radiation, also referred to as a fluorophore. A fluorescent signal is produced by a protein, for example, when the protein is capable of being excited by a particular wavelength of light and emits another wavelength of light that is detectable. The fluorescent entity can be, e.g., a protein, a lanthanide (e.g. Tb³⁺), a quantum dot (Michalet et al. Science. 2005 307(5709):538-44), or small molecule, such as green fluorescent protein (GFP), YFP (yellow) and RFP (red) (e.g. as tags), other auto-fluorescent proteins, e.g. flavins, NADH, NADPH, elastin, collagen, lipofuscin, and small molecules (as tags or dyes, including SNAP-tag (NEB), HaloTag (Promega), FlAsH (Invitrogen)), such as xanthene derivatives (fluorescein (FITC), rhodamine (TRITC), Oregon green, eosin, and Texas red), cyanine derivatives (cyanine, indocarbocyanine, oxacarbocyanine, thiacarbocyanine, and merocyanine), naphthalene derivatives (dansyl and prodan derivatives), coumarin derivatives, oxadiazole derivatives (pyridyloxazole, nitrobenzoxadiazole and benzoxadiazole), anthracene derivatives (anthraquinones, including DRAQ5, DRAQ7 and CyTRAK Orange), pyrene derivatives (e.g. cascade blue), oxazine derivatives (Nile red, Nile blue, cresyl violet, oxazine 170), acridine derivatives (proflavin, acridine orange, acridine yellow), arylmethine derivatives (auramine, crystal violet, malachite green), tetrapyrrole derivatives (porphin, phthalocyanine, bilirubin), including but not limited to the following dye families (e.g. linked to lysine or cysteine, amino or thioether bonds): CF dye (Biotium), DRAQ and CyTRAK probes (BioStatus), BODIPY (Invitrogen), ALEXA FLUOR (Invitrogen), DYLIGHT FLUOR (Thermo Scientific, Pierce), ATTO and TRACY (Sigma Aldrich), FLUOPROBES (Interchim), ABBERIOR Dyes (Abberior), DY and MEGASTOKES Dyes (Dyomics), SULFO CY dyes (Cyandye), HILYTE FLUOR (AnaSpec), SETA, SETAU and SQUARE Dyes (SETA BioMedicals), QUASAR and CAL FLUOR dyes (Biosearch Technologies), SURELIGHT Dyes (APC, RPE, PerCP, Phycobilisomes)(Columbia Biosciences)), APC, APCXL, RPE, BPE (Phyco-Biotech). A “detectable moiety” also refers to an entity that is affected by a magnetic field such as a ferromagnetic (iron, cobalt and nickel) or paramagnetic (e.g. aluminum, magnesium, molybdenum, lithium, tantalum or platinum) material.

An “engineered protein” includes any polypeptide encoded by a recombinant nucleic acid.

A “gel microdrop” or “droplet” as used herein generally comprises a limited permeability material (usually in an aqueous solution) and can be prepared, e.g. by dispersion of the limited permeability material in a second phase, such as a non-aqueous (e.g. oil) phase to form an emulsion or, alternatively, through non-emulsion based methods described herein. The limited permeability material can be present in any three dimensional shape, but typically the material is roughly spherical in shape, e.g., a microdrop. The microdrop may range from about 1 micron to about 1,000 microns in diameter. Typically the microdrop ranges from about 10 microns to about 100 microns. Ideally, the microdrop is slightly larger than the encapsulated entities (e.g. a mammalian cell is typically 10 microns or more and yeast cells are typically 4 microns or more) but not larger than suitable for the assays conducted with the microdrop. For example, if FACS is used to sort microdrops the microdrop ideally is no larger than 100 microns to allow efficient cell sorting. The microdrop may have a volume of from about 4 femtoliters to about 4 microliters. Typically, the micodrop has a volume from about 4 picoliters to about 4 nanoliters As used herein, a distinct volume of a limited permeability material may be termed a gel microdrop, a unit, or a particle, or other term understood by one of ordinary skill in the art. Suitable microdrops typically contain one or more secretory entities and/or target entities. The microdrop can be formed using a variety of methods. Such methods include but are not limited to suspension of the secretory and/or target entities in an aqueous, liquid solution of monomer capable of forming a limited permeability material (e.g. agarose, alginate, PEG, gelatin, etc.) and then adding the aqueous solution to a mixture of an oil (such as, e.g. mineral oil, hexadecane, corn oil, etc.) and surfactant (e.g. Span, sodium stearate, dodecylbenzenesulfonate, Tween, Triton, SDS, CHAPS, NP-40, among others). The aqueous polymer solution is then emulsified within the oil/surfactant layer using a variety of methods such as agitation, sonification, droplet formation, passing through a porous filter, or sorting/spotting through the use of microfluidic devices. A hydrogel can then be formed upon polymerization of the monomers, e.g., by changing the temperature of the monomer, adding an additional reagent to the aqueous solution, irradiating the aqueous solution with photons, or subjecting the aqueous droplets to a mechanical stimulus such as compression. Alternatively, the hydrogel microdrop can be formed by spotting the liquid monomeric material onto a substrate using a microdroplet generator (e.g. vibrating nozzle, microfluidic device, FACS, sonicator, etc.) and then allowing the droplet to polymerize by changing the temperature, adding an additional reagent, irradiating the droplet with photons, or through a mechanical stimulus. A macroscopic “slab” of hydrogel may be used to encase the secretory entity and/or target entity which is then separated into smaller pieces after gelling through agitation, sonication, shearing, cutting, or tearing. A collection or library of microdrops can be contained in a larger volume, which may be a liquid, semi-liquid, gel, or similar material suitable for use as provided herein. The liquid may be miscible or immiscible with water. Furthermore, the microdrops may also be encased or emulsified in a hydrophobic or hydrophilic continuous phase using a variety of surfactants to form and stabilize the emulsions. Microfluidic methods (e.g. hydrodynamic flow focusing, single-step or double-step emulsion techniques, water-in-water emulsions, water-in-oil emulsions, etc.) and microfluidic apparatuses (e.g. flow focusing devices, T-junction systems, co-axial capillary systems, micro-nozzle cross-flow systems, etc.) and suitable conditions that can be used to generate gel microdrops or droplets are described e.g. in Velasco D. et al., Small, 2012, 8 No. 11, 1633-42; and Selimovic S, Polymers, 2012, 4, 1554-79, which are incorporated herein in their entirety, and are well known in the art.

The term “induced” with respect to a cell such as a target entity or a secretory entity (e.g. a yeast cell), is intended to encompass the production of a polypeptide encoded by a nucleic acid sequence present in the cell (either a native or a recombinant nucleic acid), as well as an increase in the rate of production of the polypeptide, compared to an uninduced state. The term “induced” with respect to a promoter, is intended to encompass both the initiation of transcription of a downstream nucleic acid sequence, as well as an increase in the rate of transcription of a downstream nucleic acid sequence that is already being transcribed, compared to an uninduced state.

As used herein the term “isolated”, refers to a secretory entity, target entity, targeting moiety, target moiety, microdrop, polypeptide/protein, nucleic acid (DNA, RNA), limited permeability material (including monomers and polymers) or other material of interest that is at least 60% free, at least 75% free, at least 80% free, at least 85% free, at least 90% free, at least 95% free, at least 97% free, at least 98% free, and even at least 99% free from other components with which the entity, microdrop, polypeptide/protein, nucleic acid (DNA, RNA) or material is associated with prior to purification. The term “isolating” includes a process or method comprising one or more steps to bring about an isolated secretory entity, target entity, targeting moiety, target moiety, microdrop, polypeptide/protein, nucleic acid (DNA, RNA), limited permeability material (including monomers and polymers) or other material of interest.

A “limited permeability material” as used herein is a material that is variously permeable to biological materials contained within it and/or contacted with it, based on characteristics such as size, charge, diffusibility, and the like. In some embodiments, in a limited permeability material the ability of a secretory entity and a target entity to move through (or permeate) the material is substantially limited. In some embodiments, diffusion of the secretory entity and a target entity out of the limited permeability material contained in a microdrop is so limited that during the course of the assays to be performed on the secretory entity and the target entity neither entity migrates out of the limited permeability material. The limited permeability material is permeable to a targeting moiety secreted from the secretory entity. Where the targeting moiety is a secreted polypeptide (e.g. an antibody), a limited permeability material having a porosity between about 10 nm and up to about 1 μm is advantageous. Pore sizes of the limited permeability material permit diffusion of molecules of up to 1,000 kDa. Smaller pore sizes may permit diffusion of molecules of up to 500 kDa or up to 250 kDa. Larger pore sizes may also be used, e.g. those permitting molecules of over about 1,000 kDa to diffuse freely. Suitable limited permeability materials include hydrogels, meaning a class of highly water-absorbent (containing 90% or more water) polymeric chains or colloidal gels. Natural hydrogel materials include agarose, hyaluronan, chitosan, fibrin, alginate, collagen, gelatin, cellulose, methylcellulose, and derivatives of these materials. Other hydrogels include polyvinyl alcohol, poly(N-vinyl-2-pyrrolidone), polyethylene glycol, poly(hydroxyethyl methacrylate), acrylate polymers and sodium polyacrylate, polydimethyl siloxane, cis-polyisoprene, Puramatrix™, poly-divenylbenzene, polyurethane, and polyacrylamide among derivatives of these materials and other polymers. A polymer matrix comprises polymerized monomers capable of forming a limited permeability material, e.g. the monomers are capable of polymerizing to form such material, where the monomers were triggered to polymerize upon an external cue, such as a change in ambient temperature, contacting with a polymerization-inducing chemical or enzymatic agent, or exposure to electromagnetic radiation, such as UV or visible light. The limited permeability material can be triggered to dissolve or disintegrate (e.g. de-polymerize) by any suitable means, including by physical means (e.g. melting), chemical means (e.g. the addition of a chemical reagent that causes the dissolution, de-polymerization or increased permeability of the limited permeability material), biological means (e.g. the addition of an enzyme that degrades the limited permeability material), or other means.

The term “nucleic acid” encompasses DNA, RNA, single stranded or double stranded and chemical modifications thereof. The terms “nucleic acid” and “polynucleotide” are used interchangeably herein. The term “operably-linked” refers to the association of nucleic acid sequences on a single nucleic acid fragment so that the function of one is affected by the other. For example, a promoter is operably-linked with a coding sequence when it is capable of affecting the expression of that coding sequence (i.e., the coding sequence is under the transcriptional control of the promoter). “Unlinked” means that the associated genetic elements are not closely associated with one another and the function of one does not affect the other.

The terms “polypeptide” and “protein”, used interchangeably herein, refer to a polymeric form of amino acids of any length (usually more than 5 amino acid residues, preferably more than 10 amino acid residues), which can include coded and non-coded amino acids, chemically or biochemically modified or derivatized amino acids, and polypeptides having modified peptide backbones. The term includes fusion proteins, including, but not limited to, fusion proteins that are heterologously expressed, fusions with heterologous and homologous leader sequences, with or without N-terminal methionine residues; immunologically tagged proteins; fusion proteins with detectable fusion partners, e.g., fusion proteins including as a fusion partner to a fluorescent protein or small molecule, beta-galactosidase, luciferase, and the like. Polypeptides may be of any size, and the term “peptide” generally refers to polypeptides that are 2-25 residues in length.

The term “removing” means to take out or take away. As such, of a plurality of entities one or more entities are taken away, e.g. if the plurality of entities are in solution or in a defined space, one or more entities are taken away from the defined space or taken out of solution. For example, in a microdrop comprising limited permeability material, unbound targeting moieties secreted by the secretory entity, e.g. antibodies that do not specifically bind to a target moiety of a target entity are removed by permeating through the limited permeability material (which is permeable for the antibody) and diffusing out of the microdrop. The diffusion can be accelerated and increased, e.g. by washing the microdrop in a washing solution that readily permeates the limited permeability material and flushes out the unbound targeting moiety (e.g. the antibody). Similarly, unbound detection entities (e.g. a fluorescently labeled antibody) may be removed from the microdrop by washing the microdrop in a washing solution that readily permeates the limited permeability material and flushes out the unbound detection entity.

A “secretory entity” generally is a cellular entity such as a prokaryotic cell, e.g. a bacterium, or a eukaryotic cell, e.g. a yeast cell or a B cell, or a cell from another multi-cellular organism that is capable of secreting or releasing one or more targeting moieties. A secretory entity also includes non-cellular entities, such as a phage or other viral particle, a ribosomal complex, or a complexed entity that secretes targeting moieties upon a modification, such as, e.g. cleavage of a linker that connects the targeting moieties to each other or to a solid surface (such as a polystyrene bead). Linker cleavage may occur through enzymatic or chemical activity or may be triggered by photons, e.g. if photosensitive linkers are used. A particularly suitable secretory entity is a yeast cell.

A “separation moiety” refers to an entity that is useful to separate a secretory entity (e.g. a yeast cell), a target entity (e.g. a target bearing cell) or a microdrop containing the secretory entity and/or the target entity from one or more associated components or the environment surrounding the respective entity or the material. Typical separation moieties include magnetic particles, and moieties suitable for flow cytometer separation, plate/colony pickers, or sedimentation and centrifugation separation methods.

A “solid surface”, as used herein, includes any suitable surface on which targeting moieties or target moieties may be placed or positioned, such as a hydrophobic polymer surface (e.g. polystyrene) or a hydrophilic polymer surface (e.g. dextran), or surfaces coated with cross-linking agents, and other solid surfaces, e.g. glass, plastic or metal. The solid surface may have any shape, but preferably is a bead, but may also be a planar surface, e.g. a chip.

The term “specific binding” refers to the ability of a targeting moiety, such as an antibody, to preferentially bind to a particular target moiety, such as an epitope (e.g. an antigenic fragment) of a G protein coupled receptor, a transporter or ion channel protein, that is present in a mixture of different potential targets (e.g. a mixture of different antigens). In certain embodiments, a specific binding interaction will discriminate between desirable and undesirable target moieties in a sample. In some embodiments, specific binding by a targeting moiety to a target moiety will be more than about 10 to 100-fold or more (e.g., more than about 1000- or 10,000-fold) prominent than that of binding to a non-target moiety. In certain embodiments, the affinity between a targeting moiety and a target moiety when they are specifically bound in a targeting moiety/target moiety complex is characterized by a K_{D} (dissociation constant) of less than 10⁻⁶ M, less than 10⁻⁷ M, less than 10⁻⁸ M, less than 10⁻⁹ M, less than 10⁻¹⁰ M, less than 10⁻¹¹ M, or less than about 10⁻¹² M or less. High affinity interactions between a targeting moiety and a target moiety include K_{D} (dissociation constant) of less than 10⁻⁹ M, less than 10⁻¹⁰ M, less than 10⁻¹¹ M, less than 10⁻¹² M or less than about 10⁻¹³ M or less.

A “target entity” generally is a cellular entity such as a eukaryotic cell that exhibits, usually on its surface one or more target moieties. Cellular target entities include mammalian cells, vertebrate cells, and invertebrate cells. Particularly suitable cells are human cells. In some instances, the cells are healthy or normal cells, in other instances the cells are neoplastic or atypical cells. In some instances that cells are transformed or transfected and comprise recombinant nucleic acids, e.g. recombinant nucleic acids that encode one or more target moieties (or other engineered polypeptide target complexes) for expression and display by the target entity. In some cases the cells are transiently transfected or stably transfected cell lines. Target entities also include non-cellular entities, e.g. entities that display target moieties on a solid surface, such as a bead.

A “target moiety” is or comprises one or more epitopes that are recognizable by a targeting moiety, such as an antigen for an antibody. The targeting moiety may exhibit a certain specificity (or affinity) for the target moiety and is capable of specific binding to the target moiety. Other targeting moieties may only non-specifically interact with the target moiety or not interact at all. Particularly suitable target moieties are selected from sequences that comprise, e.g. epitopes/antigens, derived from cell membrane associated polypeptides. Cell membrane associated polypeptides that exhibit particularly suitable target moieties include ion channel proteins, transporter proteins, and G protein coupled receptors (GPCR). An “antigen,” as used herein means an entity that is capable of being specifically recognized by a targeting moiety, such as an antibody. An epitope is an antigenic determinant of a target moiety and comprises the molecular region (usually specific linear and/or spatially composed amino acid sequences) on the surface of an antigen that is capable of being specifically recognized by a targeting moiety, such as an antibody.

“Targeting moieties” as used herein are produced by the secretory entities. Particularly suitable targeting moieties are polypeptides. Targeting moieties may also include peptide, DNA, RNA, and XNA (PNA, LNA, GNA, TNA) aptamers. Targeting moieties further include small molecules that can interact, e.g. as ligands, with cell surface receptors and other extra-cellular structures, as well as lipids. Polypeptide targeting moieties preferably are antibodies or antibody-like polypeptides. Polypeptide targeting moieties can also be ligands, e.g. to cell surface receptors, such as transferrin, insulin, EGF, etc, and lipoproteins. Antibody-like proteins include alternative scaffolds that bind to target antigens. The terms “antibody” and “immunoglobulin” are used interchangeably herein. These terms refer to a protein consisting of one or more polypeptides that specifically binds an antigen. One tetrameric form of antibody constitutes the basic structural unit of an antibody, including two identical pairs of antibody chains, each pair having one light and one heavy chain. In each pair, the light and heavy chain variable regions are together responsible for binding to an antigen, and the constant regions are responsible for the antibody effector functions. The recognized immunoglobulin polypeptides include the kappa and lambda light chains and the alpha, gamma (IgG₁, IgG₂, IgG₃, IgG₄), delta, epsilon and mu heavy chains or equivalents in other species. Full-length immunoglobulin “light chains” (of, for example, about 25 kDa or about 214 amino acids) comprise a variable region of about 110 amino acids at the NH2-terminus and a kappa or lambda constant region at the carboxy-terminus. Full-length immunoglobulin “heavy chains” (of, for example, about 50 kDa or about 446 amino acids), similarly comprise a variable region (of about 116 amino acids) and one of the aforementioned heavy chain constant regions, e.g., gamma (of about 330 amino acids). The terms “antibodies” and “immunoglobulin” include antibodies or immunoglobulins of any isotype, fragments of antibodies which retain specific binding to antigen, including, but not limited to, Fab, Fv, scFv, and Fd fragments, chimeric antibodies, humanized antibodies, single-chain antibodies, and fusion proteins comprising an antigen-binding portion of an antibody and a non-antibody protein. The antibodies may be detectably labeled, e.g., with a detectable moiety (e.g., a radioisotope, an enzyme that generates a detectable product, a fluorescent protein or small molecule, a magnetic particle, and the like as provided herein). The antibodies may be further conjugated to other moieties, such as members of specific binding pairs, e.g., biotin (member of biotin-avidin specific binding pair), and the like. The antibodies may also be bound to a solid support, including, but not limited to, polystyrene plates or beads, and the like. Also encompassed by the term are Fab′, Fv, F(ab′)2, and or other antibody fragments that retain specific binding to antigen, and monoclonal antibodies. Antibodies may exist in a variety of other forms including, for example, Fv, Fab, and (Fab′)2, as well as bi-functional (i.e. bi-specific) hybrid antibodies (e.g., Lanzavecchia et al., Eur. J. Immunol. 17, 105 (1987)) and in single chains (e.g., Huston et al., Proc. Natl. Acad. Sci. U.S.A., 85, 5879-5883 (1988) and Bird et al., Science, 242, 423-426 (1988), which are incorporated herein by reference). (See, generally, Hood et al., “Immunology”, Benjamin, N.Y., 2nd ed. (1984), and Hunkapiller and Hood, Nature, 323, 15-16 (1986),). An immunoglobulin light or heavy chain variable region consists of a “framework” region (FR) interrupted by three hypervariable regions, also called “complementarity determining regions” or “CDRs”. The extent of the framework region and CDRs have been precisely defined (see, “Sequences of Proteins of Immunological Interest,” E. Kabat et al., U.S. Department of Health and Human Services, (1991). As used herein, the term “humanized antibody” or “humanized immunoglobulin” refers to a non-human (e.g., mouse or rabbit) antibody containing one or more amino acids (in a framework region, a constant region or a CDR, for example) that have been substituted with a correspondingly positioned amino acid from a human antibody. In general, humanized antibodies produce a reduced immune response in a human host, as compared to a non-humanized version of the same antibody. It is understood that the humanized antibodies designed and produced by the present method may have additional conservative amino acid substitutions that have substantially no effect on antigen binding or other antibody functions. By conservative substitutions is intended combinations such as those from the following groups: gly, ala; val, ile, leu; asp, glu; asn, gln; ser, thr; lys, arg; and phe, tyr. Amino acids that are not present in the same group are “substantially different” amino acids.

Methods for Screening and Isolating Targeting Moieties.

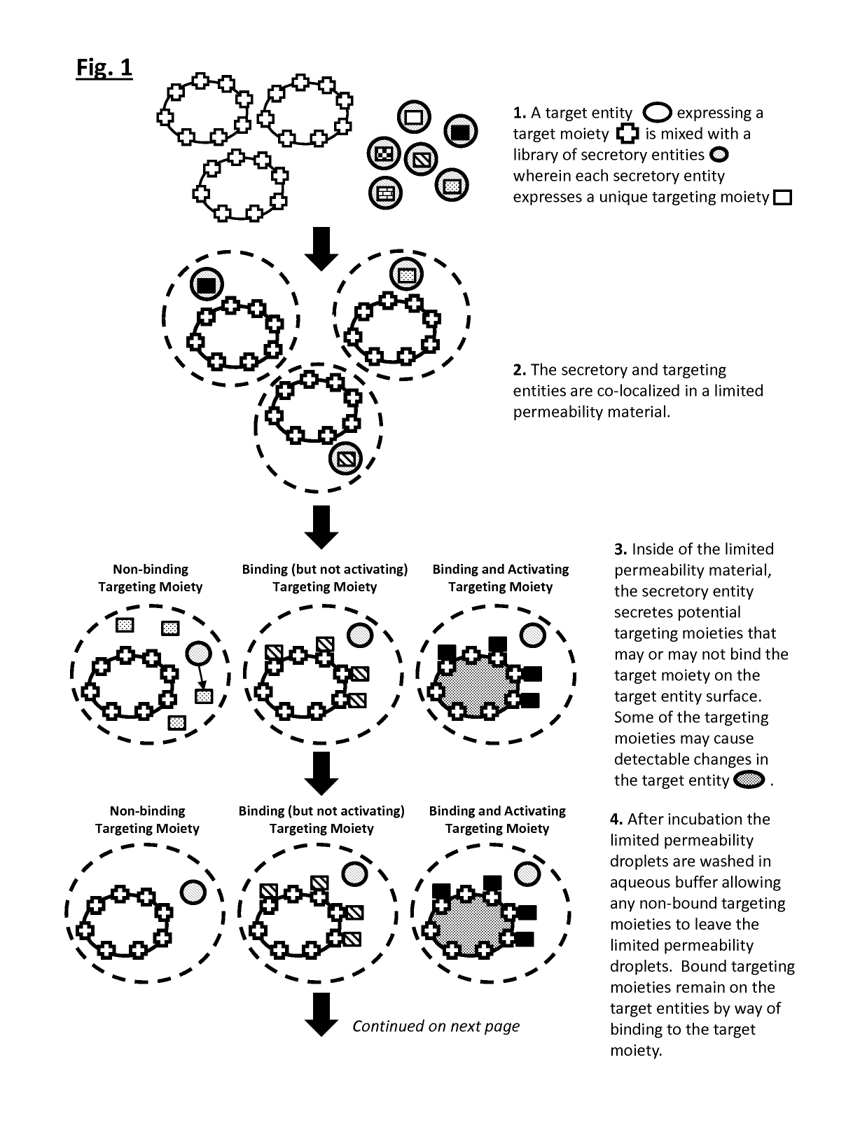

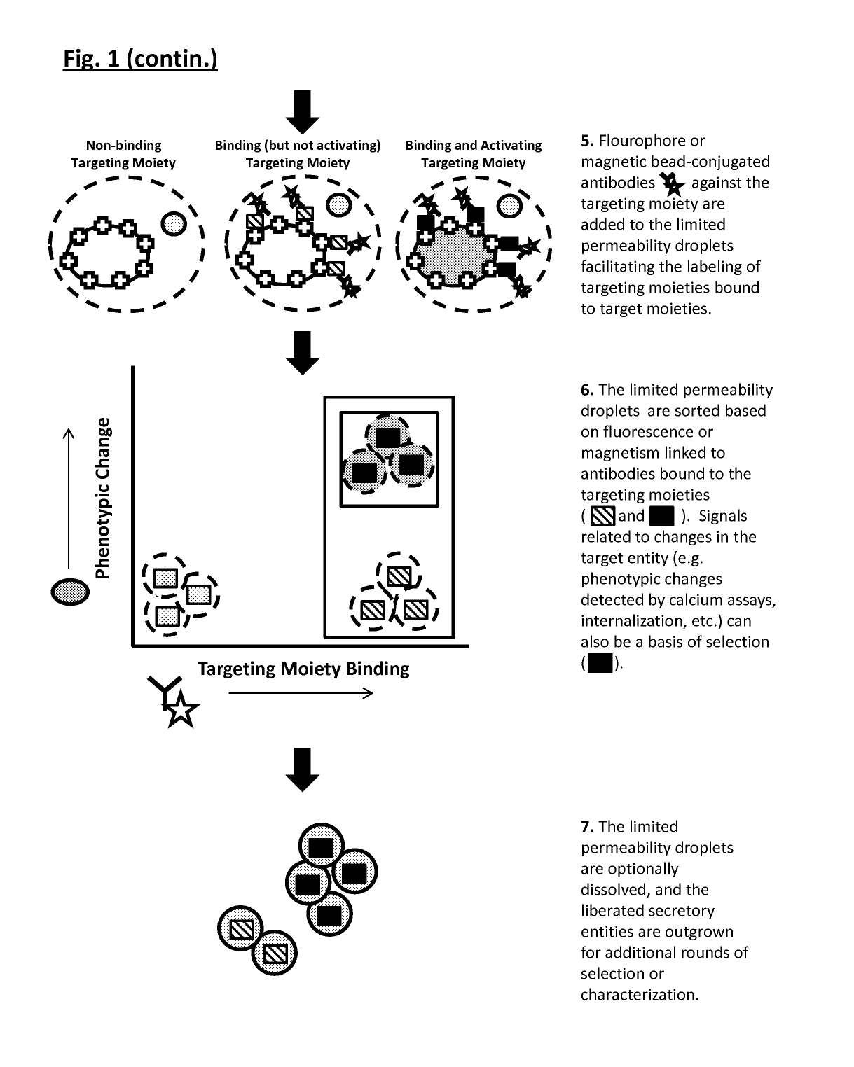

Aspects of the invention relate to methods for screening and isolating targeting moieties. In certain embodiments, methods for isolating a targeting moiety with high affinity to a target moiety from a library of targeting moieties comprise: a) mixing a target entity that exhibits a target moiety on its surface with a library of secretory entities, wherein each secretory entity produces a unique targeting moiety (FIG. 1.1); b) encasing both the target entity and the secretory entity in a limited permeability material in a microdroplet (FIG. 1.2); c) incubating the co-localized entities in the limited permeability material in the microdroplet for a time sufficient for the secretory entity to secrete a targeting moiety that may or may not bind the target moiety on the surface of the target entity, wherein some of the targeting moieties may cause detectable changes in the target entity (FIG. 1.3); d) washing the microdroplet in an aqueous buffer to remove any non-bound targeting moiety from the limited permeability material of the microdrop (FIG. 1.4); e) contacting the microdrop with a detection entity comprising a detectable moiety, such as a fluorophore or magnetic bead-conjugated antibody, wherein the detection moiety is capable of binding to the targeting moiety, to facilitate labeling of targeting moieties bound to target moieties (FIG. 1.5); f) removing a detection moiety not bound to a targeting moiety by washing the microdrop in an aqueous buffer; g) selecting a microdrop for which the detectable moiety is detected (e.g. by fluorescent or by magnetic moiety attached to the detection entity, such as an antibody), wherein if the detectable moiety is detected, the targeting moiety has affinity to the target moiety (FIG. 1.6), signals related to changes in the target entity (e.g. phenotypic changes detected by calcium assays, internationalization, etc.) can also be the basis of selection (FIG. 1.6); h) collecting the selected microdrop; i) isolating the secretory entity that secretes the targeting moiety with affinity to the target moiety by optionally dissolving the limited permeability material of the droplets and propagating the selected secretory entities; j) repeating steps (a) to (j) with the isolated secretory entity from step (j), and progressively selecting the microdrops with the highest signal for the detectable moiety, thereby isolating a targeting moiety with high affinity to a target moiety from a library of targeting moieties (FIG. 1.7).

The methods described herein can be performed using microdrops that comprise a single secretory entity and a single targeted entity. A microdrop composition comprising only two encapsulated types of entities (i.e. a secretory entity and a target entity) is advantageous over more complex microdrop compositions. The first advantage is that because co-encapsulation of the entities within a single microdroplet usually is aPoisson process, relying on two entities instead of three or four or more substantially increases the amount of microdrops that contain all of the entities desired for a particular method (e.g. assay, such as a screening assay). As a non-limiting example, in a typical Poisson process where the population of microdroplets contains an average of one entity per microdroplet roughly 37% of droplets will actually have a single secretory entity. Furthermore, because the target entity also obeys a Poisson distribution, only 37% of droplets will have a single target entity. The number of microdroplets that contain both a single secretory entity and a single targeted entity is the overlap of these two probabilities or roughly 15% of microdroplets. If a third entity is added that is to be encapsulated at exactly one entity per microdroplet, the number of microdroplets that have exactly one of each type of entity is only 5% of the population. The addition of a fourth entity reduces the likelihood to less than 2%. Consequently, limiting the number of entities to two increases the effective library size when one of the entities is diverse as well as increases the throughput of productive microdroplets about 3-fold over the number of desired microdrops that can be produced and screened if an additional entity is required. Another advantage of having one type of secretory entity and one type of targeted entity is that it greatly facilitates selection by high-throughput methods such as FACS or magnetic bead selection. FACS analyzes properties of the microdrop as a whole and cannot give information about signal distribution within the microdrop. For example, in a microdrop containing only a secretory entity and a target entity (e.g. a yeast cell secreting an antibody and a mammalian cell comprising a cell-surface protein), the FACS can measure the retention of the targeting moiety (e.g. the antibody) by analyzing the retention of the detection moiety which is retained within the microdroplet by binding the targeting moiety which is retained within the droplet by binding the target moiety. The FACS does not measure if the antibody is retained on the mammalian surface, only the detectable moiety is present inside of the microdroplet. In many embodiments, it is not important that one identify exactly where in the microdroplet the targeting moiety binds. For this reason, FACS becomes a screening option thus greatly increasing throughput. In situations where two targeted entities are required (for example a positive control bead bearing the target moiety and a negative control bead that lacks the target moiety), it is critical for the success of the assay that the accumulation of the targeting moiety on the positive control bead and not on the negative control bead can be visualized. Consequently, a lower throughput, less quantitative method such as microscopy must be used for selection. In situations where two different targeted entities are used (e.g. a positive control and negative control bead), high-throughput methodologies such as FACS or magnetic sorting would be unable to distinguish where the targeting entities are bound and would consequently isolate everything that had retained targeting moieties whether it bound to the positive control bead or negative control bead or both. The simplified systems, microdrop compositions and methods described herein greatly accelerate through-put and increase the library sizes able to be screened enabling a large diversity of libraries such as large naïve, hybridoma, immune derived antibody libraries as well as genomic and cDNA libraries which tend to have ten million or more members. It is also significant that in generally the methods and compositions described herein utilize a singular target entity that is distinct from the limited permeability material instead of a, e.g. target entity that is distributed throughout or even a part of the limited permeability material. The most significant advantage of this distinction is that target moieties in their native, cellular context can be used. A great many of the most interesting drug targets are multi-pass transmembrane receptors such as GPCRs, ion channels, and transporters that lose fidelity and structure when removed from their cellular context. Consequently, expressing and purifying these target moieties recombinantly so that they may be distributed throughout the limited permeability material while retaining native structure is not easily done and may in many instances not be feasible at all. To distribute target moieties throughout the limited permeability material, they must first be expressed and purified. Additionally, they are frequently further modified, e.g., through biotinylation or some other modification motif in order to be immobilized in the limited permeability material. The immobilization frequently relies on a non-covalent interaction with the limited permeability material which means that the target moieties may dissociate from the limited permeability material thus affecting their availability to bind to the targeting entity. The methods and microdrop compositions described herein eliminate many of the expression, purification, modification, immobilization, and retention limitations of other methods. In certain embodiments, the target moiety is not an extracellular domain of a protein. In these instances, extracted intracellular material can be immobilized, e.g., on a functional bead such as a DYNAL Epoxy bead which can then be used as a target entity using methods described herein.

Microdrops and Mammalian Cell Complexes.

Provided herein are multifactorial units such as microdrops useful in the methods described herein, which contain one or more target entities (e.g. mammalian cells), one or more secretory entities (e.g. yeast), and a medium or material that encapsulates or encases the target entities (e.g. mammalian cell(s)) and the secretory entity(ies) (e.g. yeast). Gel microdrops comprising a limited permeability material, a secretory entity and a target entity that is a mammalian cell are also referred to herein as “mammalian cell complexes.” Typically, this material is a “limited permeability material”, meaning that material is variously permeable to biological materials contained within it and/or contacted with it, based on characteristics such as size, charge, diffusibility, and the like. In some embodiments, in a limited permeability material the ability of a target entity such as a mammalian cell to move through (or permeate) the material is substantially limited. For example, the target entity (e.g. mammalian cell) is capable of moving less than one entity (e.g. cell) diameter per unit time, e.g., 1, 2, 3, 4, 5, 10, 15, 30, 45, or 60 minutes, or 1, 2, 3, 4, 5, 6, 7, 8, 9, 10 or more than 10 hours. The limited permeability of a mammalian cell in a limited permeability material is at least in part a factor of the type of mammalian cell, e.g., whether that cell is typically invasive (such as a tumor cell or a leukocyte). In some embodiments, in a limited permeability material the ability of a secretory entity to move through the material is also substantially limited. As provided herein, a secretory entity is a prokaryotic cell such as, e.g. a bacterium or a eukaryotic cell such as, e.g. a yeast cell or a cell from a multi-cellular organism. Alternatively, a secretory entity is a non-cellular material, such as a phage or other viral particle, or a ribosomal complex. A suitable secretory entity is a yeast cell. For example, the secretory entity is capable of moving less than one entity diameter per unit time, e.g., 1, 2, 3, 4, 5, 10, 15, 30, 45, or 60 minutes, or 1, 2, 3, 4, 5, 6, 7, 8, 9, 10 or more than 10 hours.

In some embodiments, the gel microdrop comprises about one target entity and one secretory entity. In other embodiments, the gel microdrop comprises exactly one target entity and one secretory entity. In some embodiments, the gel microdrop comprises one target entity and more than one, e.g. two, three, four, five or more secretory entities. In some embodiments, the gel microdrop does not contain or comprise more than one target entity that is distinct, i.e. it does not contain or comprise a first and a second target entity that are not the same, i.e. distinct from each other. In specific embodiments, provided herein are microdrops comprising a limited permeability material, a secretory entity, and a first target entity comprising a target moiety, with the proviso that the gel microdrop does not contain a second target entity that is distinct from the first target entity. In some embodiments, a gel microdrop comprises at least one target entity, at least one secretory entity and both the target entity and the secretory entity are not substantially capable of permeating through the limited permeability material. In one example, the microdrop comprises a mammalian cell complex that contains at least one mammalian cell and at least one yeast cell secretory entity, and both the mammalian cell and the yeast cell are not substantially capable of permeating through the limited permeability material. “Not substantially capable” means that, e.g., during the course of the assays to be performed on the gel microdrop (or mammalian cell complex) the yeast cell and the mammalian cell do not migrate out of the limited permeability material. The limited permeability material is produced so that it is permeable to a targeting moiety (such as a polypeptide) secreted from the secretory entity. Where the targeting polypeptide is a secreted antibody, a limited permeability material having a porosity between about 10 nm (roughly twice the radius of gyration of an antibody) and about 5 μm (roughly the diameter of a yeast cell) is advantageous. Other suitable porosities for the limited permeability material range from about 5 nm to about 5 microns, and from about 10 nm to about 2 microns, to about 3 microns, or to about 4 microns.

In some embodiments, the limited permeability material is permeable for the targeting moiety and it can freely move within the material and/or diffuse out of the material. In an embodiment, the limited permeability material does not comprise a target moiety or a targeting moiety that is linked to or bound by the limited permeability material. Specifically, the monomers or polymers and polymer chains that make up the limited permeability material are not conjugated, linked to or bound by either a target moiety or a targeting moiety. Thus, the limited permeability material in the absence of a target entity is not by itself capable of capturing a targeting moiety that is secreted from the secretory entity. Provided herein are microdrops comprising a limited permeability material, a target entity comprising a target moiety and a secretory entity capable of secreting a targeting moiety, with the proviso that the limited permeability material does not comprise a target moiety or targeting moiety that is conjugated, linked or bound to the monomers, polymers or polymer chains making up the limited permeability material. Thus, the encapsulation of the target entity and the secretory entity by the limited permeability material as well as the optional encapsulation of the limited permeability material by a non-aqueous phase (e.g. oil) to form an emulsion does not provide target moieties for the secreted targeting moieties in addition to those provided by the target entity, neither within the mesh created by polymerized monomers of the limited permeability material nor in the outside perimeter or wall created by the microdrop formation. As used herein, the “target entity” is distinct from the “limited permeability material” and is suspended therein.

In certain embodiments, mammalian cells acting as target entities are selected for the cell membrane localization of desired target moieties (such as polypeptides), for which a targeting moiety (e.g. an antibody) capable of binding specifically thereto is selected. Such a target moiety screening system is useful to isolate novel binders (such as antibodies) to cell surface proteins and transmembrane proteins, such as ion channel proteins, transporter proteins, and G protein coupled receptors (GPCR). The targeting moiety (e.g. antibody) is freely diffusible, meaning the targeting moiety (e.g. antibody or antibody-like polypeptide) is capable of permeating through the limited permeability material, yet the association between the desired antibody and the encoding genotype is maintained as the target entity which presents the phenotype used as the basis for selection and the secretory entity comprising the genotype for the targeting moiety responsible for the phenotypic change in the target entity are not permeable through the limited permeability material. Consequently, isolating the microdrop (e.g. mammalian cell complex) based on either binding of targeting moiety to target moiety as reported by a detection entity or phenotypic change directly reported by detecting a change in the targeted entity itself will also isolate the gene encoding the targeting moiety as the two are spatially linked vis a vie their lack of permeability in the limited permeability material in the droplet.

The limited permeability material can be present in any three dimensional shape, but typically the material is roughly spherical in shape, e.g., a microdrop. As used herein, a distinct volume of a limited permeability material may be termed a microdrop, a unit, or a particle, or other term understood by one of ordinary skill in the art. The size (or volume) of the microdrop comprising the limited permeability material containing the target entity (e.g. mammalian cell) and secretory entity (e.g. yeast cell) is at least in part dependent upon the means of detecting the interaction between a given target moiety (e.g. an antigen) on the target entity (e.g. mammalian cell) and the targeting moiety (e.g. a polypeptide, typically an antibody) as well as limitations of the separation moiety. A volume for sorting the microdrops (or mammalian cell complexes) by flow cytometry below about 100 μm in diameter is generally suitable, such as 100 μm, 75 μm, 50 μm, 25 μm, 15 μm, 10 μm, or less than 10 μm.

Suitable limited permeability materials include hydrogels, meaning a class of highly water-absorbent (generally containing 90% or more water) polymeric chains or colloidal gels. Natural hydrogel materials include agarose, hyaluronan, chitosan, fibrin, alginate, collagen, gelatin, cellulose, methylcellulose, and derivatives of these materials. Other hydrogels include polyvinyl alcohol, poly(N-vinyl-2-pyrrolidone), polyethylene glycol, poly(hydroxyethyl methacrylate), acrylate polymers and sodium polyacrylate, polydimethyl siloxane, cis-polyisoprene, Puramatrix™, poly-divenylbenzene, polyurethane, and polyacrylamide among derivatives of these materials and other polymers. Often hydrogels can be formed by cross-linking polymeric chains such as in the cross-linking of polypeptide chains with Factor XIII or transglutaminase.

A collection of microdrops may be contained in a larger volume, which may be a liquid, semi-liquid, gel, or similar material suitable for use as provided herein. The liquid may be miscible or immiscible with water. Furthermore, the microdrops may also be encased or emulsified in a hydrophobic or hydrophilic continuous phase (as the larger volume) using a variety of surfactants to form and stabilize the emulsions. The microdrops can be used as individual reaction vessels to perform binding reactions between the targeting moiety secreted from the secretory entity and the target moiety displayed by the target entity which are all located in the limited permeability material that makes up the microdrop. For example, once a yeast cell is induced to produce and secrete a targeting moiety (e.g. an antibody) it diffuses through the limited permeability material of the microdrop until it contacts the target moiety displayed on the target entity, such as a mammalian cell. If the antibody binds to the mammalian cell, then it becomes localized on the mammalian cell. If the antibody does not bind the mammalian cell, then it is free to diffuse out of the microdrop and into the surrounding space.

In exemplary mammalian cell complexes, one microdrop contains one yeast cell and one mammalian cell. A diverse antibody library introduced into a population of yeast cells is then distributed (or assigned) to individual microdrops. Preferably, each microdrop contains a single secretory entity (e.g. yeast cell), although in some instances the distribution of more than one secretory entity (e.g. yeast cell) per microdrop is desired. For example, in a given microdrop is one target entity (e.g. a mammalian cell) and between 2, 3, 4, 5, 6, 7, 8, 9, 10 or more than 10 secretory entities (e.g. yeast cells). Alternatively, the microdrop contains a ratio of secretory entities to target entities (e.g. yeast cells to mammalian cells) of about 10:1, 5:1, 4:1, 3:1, 2:1, 1:1, 1:2, 1:3, 1:4, 1:5, or 1:10.

Detection of Targeting Moieties.

Following induction of the production of the targeting moiety (e.g. an antibody, typically an IgG) and its secretion from the secretory entity (e.g. yeast cell), the populations of microdrops are washed and then contacted with a detection entity comprising a detectable moiety, e.g., a fluorophore-labeled anti-IgG, which binds to the targeting moiety (e.g. targeting antibody) specifically localized on the surface of the target entity (e.g. mammalian cell) after binding to the target moiety. Fluorescence of a given microdrop indicates that a targeting moiety (e.g. antibody) has accumulated on the surface of the target entity (e.g. mammalian cell), allowing the sorting of the microdrop by flow cytometry. Alternatively, where there is no detectable accumulation of targeting moiety (e.g. antibody) on the surface of the target entity (e.g. mammalian cell), the microdrop does not fluoresce and is not be sorted. Typically, the secretory entity (e.g. yeast cell) embedded in a fluorescent microdrop is sorted by either flow cytometry or magnetic particle selection. Following selection, viable secretory entities such as yeast cells are isolated by optionally dissolving the microdrops (dissolving or de-polymerizing the limited permeability material) and expanding the pool of selected secretory entities (e.g. yeast cells) which can then be characterized. The selection process described herein may be repeated one or more times, such as 2, 3, 4, 5, 6, 7, 8, 9, 10 or greater than 10 times in order to select high affinity binding targeting entities, such as antibodies.

In certain embodiments, a population of secretory entities (e.g. yeast cell clones) collectively containing a diverse targeting moiety (e.g. antibody) library are introduced into microdrops with target cells (e.g. mammalian cells or cell lines) that lack the target moiety (e.g. antigen), such that the targeting moieties (e.g. antibody or antibody-like polypeptide) can interact and specifically bind an antigen other than the target moiety. This is useful in order to deplete from the population of secretory entities (e.g. yeast cells) those entities that produce targeting moieties (e.g. antibodies) that bind to non-target antigen(s).

Detection of the bound targeting moieties (e.g. antibodies) may be performed using a detection entity, wherein the detection entity is capable of binding specifically the targeting moiety (e.g. antibody) and contains a detectable moiety. Optionally, the targeting moiety (e.g. antibody or antibody-like polypeptide) contains a detection tag, and the detection entity is capable of binding to the detection tag. For example, detection tags such as FLAG, myc, His, V5, and the human Fc can be used as there are a number of antibodies against them (some of which are tagged with a detection moiety) which can be used to detect their presence.

The interaction between enzymes and their substrates can be determined using the methods herein. For example, soluble enzymes acting as targeting moieties are secreted from secretory entities (e.g. yeast cells) and may interact with a target moiety that is a substrate of the enzyme and is displayed on the surface of the target entity (e.g. a mammalian cell-membrane associated substrate). Alternatively, a soluble substrate or ligand acts as the targeting moiety and is secreted from the secretory entity and the enzyme acting as the target moiety is present on the surface of the target entity (e.g. mammalian cell). For example, a yeast population containing one or many enzyme-encoding nucleic acids is introduced into the microdrops, wherein the mammalian cell contains a substrate.

Charged polypeptides are known in the art to have cell-penetrating, stabilizing and anti-aggregative properties. However, it is often difficult to screen such charged polypeptides (e.g., supercharged polypeptides) in a meaningful way using yeast or bacteria expression systems. In additional embodiments, a population of yeast cell clones collectively containing a diverse supercharged polypeptide library is introduced into microdrops with mammalian cells, and the intracellular localization of any such cell-penetrating supercharged polypeptide in the target mammalian cell is determined. Alternatively, in situations where the cell-penetrating supercharged polypeptides detectably modify the target mammalian cell, such modification is determined and evaluated.

Methods of Protein Display.

Aspects of the invention relate to methods for the display of a target moiety such as a polypeptide or a polypeptide complex on a target entity such as a mammalian cell. For example, a mammalian cell is provided in a microdrop comprising a limited permeability material, along with a secretory entity, such as a yeast cell or other entity (e.g. an entity that contains a nucleic acid that encodes an engineered polypeptide), to form a mammalian cell complex, and the mammalian cell complex is incubated under conditions sufficient to express and secrete the targeting moiety (e.g. an engineered polypeptide) by the secretory entity. Upon binding of the target entity vis a vie the target moiety (e.g. a mammalian cell vis a vie a receptor expressed on the cell's surface) by the targeting moiety (e.g. engineered polypeptide) a secreted engineered protein complex comprising the target moiety and targeting moiety is displayed on the target entity (e.g. mammalian cell) which can be detected and selected as described herein.

Also provided are methods of selecting a target entity (e.g. mammalian cell) that contains at its surface a target moiety (e.g. polypeptide), wherein the target entity is present in a microdrop with a secretory entity that produces and secretes a targeting moiety (e.g. an engineered protein) that thereafter binds the target moiety. In one example, the targeting moiety (e.g. secreted engineered protein) contains a detectable moiety or is bound by a detection entity, such that a complex of the target entity (e.g. mammalian cell) and the targeting moiety (e.g. bound engineered protein) can be detected and separated using the methods described herein.

Another aspect of the invention relates to methods of screening a plurality of differentiated targeting moieties (e.g. engineered proteins) by performing the selection method described herein in a plurality of microdrops (e.g. mammalian cell complexes). Each microdrop (e.g. mammalian cell complex) within the plurality of microdrops may contain one mammalian cell that is substantially the same as the mammalian cells within the other droplets in the plurality of microdrops. Isolating microdrops with desired characteristics as measured by a detection entity can isolate targeting moieties with desired properties. An non-limiting example of this approach is the isolation of an antibody(s) against a cell-surface receptor by selecting it from a library of micrdrops each comprising a different targeting entity (e.g. antibody). Alternatively, the plurality of microdrops may collectively contain a plurality of mammalian cell complexes each comprising one or more of a variety of different target moieties with approximately one differentiated target entity (e.g. mammalian cell) within each microdrop. The targeting moieties encoded within the secretory entity are substantially the same in each micrdroplet within the plurality of microdrops. In this embodiment, selections are made for cell types that respond in a particular manner (e.g. with a phenotypic change) to a given targeting moiety. For example, multiple mammalian cell lines can be screened for responses to a single, uniform growth factor expressed by every secretory entity (e.g. yeast) within the plurality of micodrops. Microdrops isolated based on their response to the targeting moiety (e.g. growth factor) may then contain target entities (e.g. cell lines) that are responsive to the growth factor. Such a plurality of microdrops or mammalian cell complexes contains, e.g., at least about 1×10², 1×10³, 1×10⁴, 1×10⁵, 1×10⁶, or greater than 1×10⁶ microdrops or mammalian cell complexes. The targeting moiety can be an engineered protein (preferably an antibody), and at least about 1×10², 1×10³, 1×10⁴, 1×10⁵, 1×10⁶, 1×10⁷, 1×10⁸, or greater than 1×10⁸ unique targeting moieties (e.g. antibodies) are present in the plurality of microdrops or mammalian cell complexes.

One specific example relates to the selection of anti-Epithelial Growth Factor Receptor (EGFR) antibodies binding specifically to mammalian cell surface EGFR. A mammalian cell that overexpresses EGFR is encapsulated and immobilized in a microdrop with a diversified antibody library present in a yeast population, such that each microdrop contains about one mammalian cell and about one unique yeast clone from the antibody library.

Mammalian cells include human cells such as a human cancer cell or tumor cell line, as well as cell lines, e.g. cell lines that are frequently used for the overexpression of proteins such as HEK293, CHO, HeLa, etc.

The yeast can express targeting moieties other than antibodies, such as, e.g. antibody derivatives, fibronectins, DARPINs, integrins, receptor ectodomains, peptides, growth factors, or other molecules capable of being secreted and binding a target moiety.

The microdrop can be formed using a variety of methods. Such methods include but are not limited to suspending the secretory entities (e.g. yeast) and target entities (e.g. mammalian cells) in an aqueous, liquid solution of monomer (e.g. agarose, alginate, PEG, gelatin, etc.) and adding the aqueous solution to a mixture of an oil (such as, e.g. mineral oil, hexadecane, corn oil, etc.) and surfactant (e.g. Span, sodium stearate, dodecylbenzenesulfonate, Tween, Triton, SDS, CHAPS, NP-40, among others). The aqueous polymer solution is then emulsified within the oil/surfactant layer using a variety of methods such as agitation, sonification, droplet formation, or sorting/spotting through the use of microfluidic devices which are well-described in the art. For instances where the limited permeability material is a hydrogel, the hydrogel can be formed, e.g., by changing the temperature of the monomer, adding an additional reagent to the aqueous solution, irradiating the aqueous solution with photons, or subjecting the aqueous droplets to a mechanical stimulus such as compression. Alternatively, the hydrogel microdrop can be formed by spotting the liquid monomeric material onto a substrate using a microdroplet generator (e.g. vibrating nozzle, microfluidic device, FACS, sonicator, etc.) and then allowing the droplet to polymerize by changing the temperature, adding an additional reagent, irradiating the droplet with photons, or through a mechanical stimulus. The microdrops can be eluted from the solid substrate by washing.

In yet another method, target entities (e.g. mammalian cells) and secretory entities (e.g. yeast cells) may be encased in a macroscopic “slab” of hydrogel which is then separated into smaller pieces after gelling, e.g., through agitation, sonication, shearing, cutting, or tearing.

Whatever the method for encapsulating target entities (e.g. mammalian cells) and secretory entities (e.g. yeast cells), the microdrop is maintained in an environment that allows the secretory entity (e.g. yeast cell) to secrete the targeting moiety (e.g. antibody). Typically, the secretory entity is a cell comprising a nucleic acid plasmid that codes for the targeting moiety maintained in the cell, such as a yeast cell possessing a gene for an antibody. In situations where an emulsion is used to form the microdroplets, the microdroplets may be maintained in the emulsion throughout the targeting moiety (antibody)-secretion process. Alternatively, the emulsion may be washed away before the incubation period. In situations where microdroplets are formed on a substrate, the incubation period may take place upon that substrate, or the microdroplets may be washed off the substrate before the incubation period. In situations where the target entity and the secretory entity are first embedded in a “slab” of gel, the incubation period may take place within that slab, or the slab may be treated in such a manner so as to create small hydrogel droplets using one of the methods described herein. Optionally, the expression of the targeting moiety (e.g. antibody) in the secretory entity (e.g. yeast cell) is induced by a chemical or environmental alteration in the microdrop such as the addition or removal of a carbon source or antibiotic.

Incubation times for secretory entities and target entities in the microdrop (or mammalian cell complex) may vary to induce expression and secretion of the targeting moiety and binding of the targeting moiety to the target moiety. Suitable incubation times vary from several minutes to several hours, e.g. about 1, 2, 3, 4, 5, 6, 7, 8, 9, 10, 20, 30, 40, 50 minutes, or about 1, 2, 3, 4, 5, 6, 7, 8, 9, 10, 12, 14, 16, 18, 20, 24, and 48 hours. After incubation, e.g. to allow for antibody secretion by the yeast cell and subsequent binding by the antibody to the mammalian cell present in the microdrop, the microdrop is washed, meaning it is contacted with a solution such that substantially any unbound antibody (targeting moiety) is removed from the microdrop. If the microdrop is maintained in the emulsion during the incubation period, the washing may include repeatedly contacting the emulsified hydrogel (as an example of a limited permeability material in the microdrop) with an oil phase in order to break the emulsion before washing the liberated microdroplets with the aqueous solution to remove the antibodies. The washing may also include washing microdroplets from a substrate upon which they were formed or making smaller microdroplets from a hydrogel slab using the methods described herein before washing with the solution to remove unbound, free targeting moiety (e.g. antibody).

The microdrops are then labeled with a detection entity that comprises a detectable moiety, such as, e.g. fluorophore-conjugated anti-human IgG. Alternatively or in addition, the microdrops can be labeled with a detection entity comprising a magnetic detectable moiety, e.g anti-human IgG conjugated to a magnetic particle. The microdrops are analyzed, e.g., by flow cytometry. Microdrops containing fluorescently detectable IgG (detection entity), for example, indicative of antibodies bound to the EGFR-expressing mammalian cell described herein, can be retained by sorting. If magnetic particles are used as detection entities, e.g. magnetic particle-conjugated IgG, a magnet can be used to separate microdrops containing detectable IgG.

After selection and separation, the limited permeability material is optionally removed through dissolution (or de-polymerization) by physical (e.g. melting), chemical (e.g. the addition of a chemical reagent that causes the dissolution or increased permeability of the limited permeability material), biological (e.g. the addition of an enzyme that degrades the limited permeability material), or other means and the secretory entities (e.g. yeast cells) that were encased in the limited permeability material are recovered. Alternatively, the limited permeability material is not dissolved. Optionally, the recovered secretory entities are propagated, e.g. to determine the nucleic acid sequence encoding the targeting moiety that is specific for the target moiety (e.g. an antibody specific for EGFR-binding as described herein). Alternatively, the secretory entities (e.g. yeast cells) are grown within the limited permeability material, and no degradation is needed. Optionally, one or more additional rounds of selection are performed.

Optionally, non-specific targeting moieties (e.g. antibodies) that do not bind to target moieties on the target entity (mammalian cell) but instead bind other surface polypeptides or cell surface biomolecules on the target entity are removed prior to selection. For example, to obtain antibodies specific for EGFR, one or more depletion rounds for non-specific targeting moieties are conducted using target entities (e.g. mammalian cells) that do not display (or express) EGFR on the surface. Microdrops that do not retain IgG are then selected (e.g. using anti-IgG-specific detectable entities) and retained so as to ensure that only antibodies against the EGFR target protein are recovered in subsequent selection rounds. This initial round or rounds with EGFR negative cells is a selection against antibody binders to other, irrelevant mammalian surface localized proteins. Preferably, the EGFR deficient cell line is of the same origin or has the same characteristics as the cell line that expresses EGFR in order to maximize the pre-screening selection against irrelevant cell surface polypeptides. Selections where both EGFR-expressing and non-EGFR expressing cell lines are available can be performed by transfecting and overexpressing the EGFR gene in a cell line that does not normally express EGFR or eliminating EGFR expression from a cell line that normally does express EGFR, e.g. through genomic deletion or alteration, expression knock-down such as RNAi, or protein-level interference such as the co-expression of an intrabody or aptamer against EGFR which prevents its surface expression.

In order to normalize sorting, in some embodiments, an irrelevant target on the target entity (e.g. mammalian cell) is bound by a detection entity (e.g. a fluorophore-tagged antibody) against a protein that is different from the target moiety targeted by the secretory entity (e.g. target antibody). This set up provides the ability to normalize for the number and/or size of the target entity (e.g. mammalian cell). Using an antibody against the secretory entity (e.g. yeast cell) allows one to normalize for the number of secretory entities present in a given microdrop. It is recognized that a poor affinity targeting moiety (e.g. antibody) can be highly present in a microdrop if there are multiple target entities (e.g. mammalian cells) each presenting target moiety (e.g. antigen), relative to a microdrop containing a single target entity. Also, it is useful to determine the presence of multiple secretory entities (e.g. yeast cells) that produce high amounts of low affinity targeting moieties (e.g. antibody) relative to a single secretory entity that produces a higher affinity targeting moiety. Thus, quantifying the number of entities (e.g. cells) present in the microdrop allows for the normalization of the retained targeting moiety (e.g. antibody) to the amount of target entities and the number of secreting entities present in the microdrop. For example, in detecting EGFR-binding antibodies, the microdrops are labeled with an antibody specific to a yeast cell surface protein, such as FLO1, which is conjugated to a first fluorophore. The microdrops are also labeled with an antibody conjugated to a second fluorophore that is specific to a non-targeted moiety on the mammalian cell (target entity). This non-targeted moiety could be a cluster of differentiation (CD) protein, a receptor, a tansporter, an ion channel, or an adhesion molecule. The only limitation in the selection of the non-targeted moiety is that binding of the antibody to the non-targeted moiety does not activate the cell in the same way as binding of the target moiety. The microdrops are further labeled with an antibody conjugated to a third fluorophore that is an anti-human IgG antibody, which detects a targeting moiety (antibody) bound to EGFR. The microdrops are sorted for high level of anti-target antibody binding (third fluorophore) relative to the amount of mammalian surface expression (second fluorophore) and number of yeast clones (first fluorophore) as determined by relative signal of the three fluorophore-conjugated antibodies.

It is possible to screen for mammalian cell binding antibodies (or other polypeptides) without a priori knowledge of the target polypeptide present on the mammalian cell. This method is useful when a cell, such as a tumor cell or cancer cell line, expresses an unknown cell surface antigen, or a plurality of insufficiently described cell surface antigens. In one embodiment, it is desirable to perform a pre-selection on non-tumor cells, such as healthy cells from the same tissue (e.g., from normal adjacent tissue) in order to remove irrelevant binding antibodies. Optionally, the screening of cell-surface associated tumor cell markers is paired with a phenotypic determination as provided herein.

In addition to detection and selection using detectable moieties such as fluorescent moieties, also provided is the use of the combination of fluorescent detectable moieties and one or more phenotypic changes on or in the target entity, such as a mammalian cell. In certain embodiments, targeting moieties (e.g. antibody binders) to a cell surface biomolecule are screened and, either simultaneously or sequentially, a screen is performed for a modified phenotypic behavior of the target entity resulting from the binding of the targeting moiety to the target moiety. By way of non-limiting example, provided is a screen for binding to a pro-apoptotic receptor with a read-out for apoptosis in order to find an antibody that functions as a receptor agonist, thereby inducing apoptosis. Screening for death receptor 6 (DR6) binding antibodies is combined with detection of apoptosis in a cell line. Apoptosis is measured by labeling the microdrop with a DNA stain such as ethidium bromide or DAPI that is only able to stain the nucleus when the cell membrane has become compromised due to apoptosis. In such a screen, a DR6 expressing mammalian cell is localized in a microdrop with unique yeast clones from an antibody library. A subset of the yeast clones secrete antibodies that bind to the DR6 expressing cell, thereby inducing an apoptotic response. It is recognized that potentially only a subset of the DR6-binding antibodies are capable of inducing a cellular response. The microdrop is then labeled with a DNA stain such as DAPI or propidium iodide and sorted by flow cytometry. Microdrops are screened for retention of anti-DR6 antibody, as measured by a fluorophore-conjugated anti-human IgG antibody, as well as for the presence of the DNA stain, indicative of an apoptotic cell.

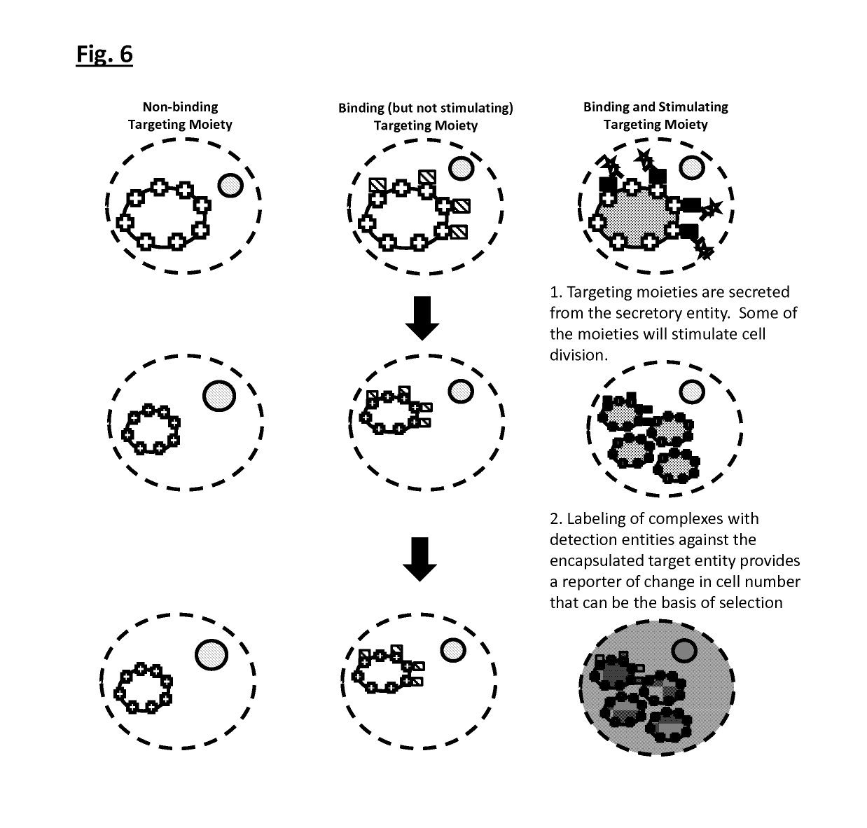

In addition, provided are the use of pluralities of microdrops or mammalian cell complexes to screen libraries such as yeast that express proteins other than antibodies, in order to identify polypeptides having agonist or antagonist behavior of cell-surface localized proteins. In some embodiments, the libraries are variants of polypeptides known or believed to have such agonist or antagonist behaviors. For example, a yeast library of variant growth factor polypeptides is combined in complex with a mammalian cell expressing on its cell surface the growth factor receptor. Selection can be performed based on mammalian cell growth or other phenotypic changes, which are monitored through the use of antibodies against phenotypic markers or other markers of the growth factor effect or measure of the proliferation of the cell itself. In an additional embodiment, yeast cells, each expressing a unique variant of targeting entity (e.g. polypeptide), are co-localized in a limited permeability microdrop with a rapidly dividing mammalian cell. Selections can be made for cells that stop dividing in the presence of the targeting moiety by isolating microdrops or mammalian cell complexes with relatively low levels of detection entities against a polypeptide on the mammalian cell surface using methods described herein (FIG. 6).

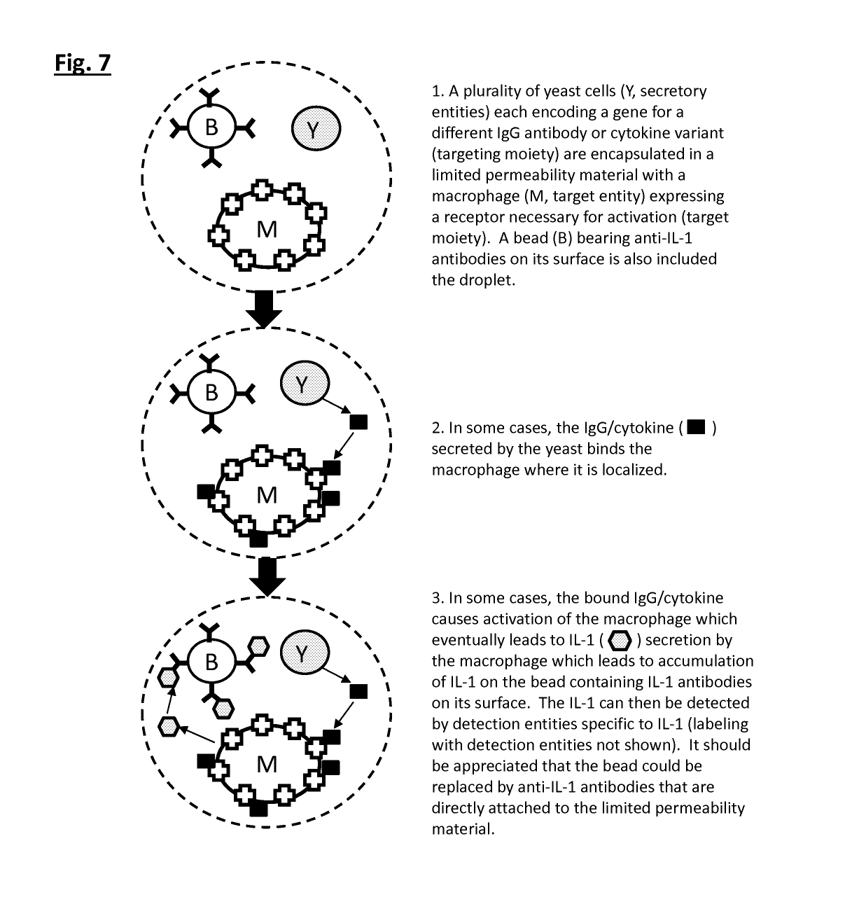

In some embodiments, selections of antibodies that bind to polypeptides secreted from a mammalian cell (secretory entity) are provided (FIG. 7). For these selections, included in the microdrop is a particle or other material that contains, preferably on its surface, an immobilized antibody to the mammalian cell secreted polypeptide. Thus, the mammalian cell secreted polypeptide is bound to this particle, and is then further bound by a targeting antibody, which is in turn detected by means provided herein. In certain embodiments, the secreted factor is made by the mammalian cell in response to a stimulus. For example, macrophages are co-localized with a yeast library and a particle displaying anti-IL-1 antibodies. When the macrophage is activated, the cell secretes IL-1, which becomes immobilized on the particle and is available to be labeled with a fluorophore-conjugated antibody. Macrophage activation is measured by assaying the fluorescence of the microdrop via an anti-IL-1 fluorophore-conjugated antibody. Antibodies that agonize macrophage activation are selected by sorting for IL-1 immobilized on the particle in addition to antibody accumulated on the mammalian cell surface. Antibodies that block macrophage activation in the presence of a normal pro-activation stimulus are selected by sorting microdrops without anti-IL1 antibody accumulation but with the accumulation of secreted antibody on the mammalian cell surface.

A great number of phenotypic changes may be used as reporters for changes in the target entity brought about by binding of the targeting moiety to the target moiety displayed on the target entity. In certain embodiments, proteomic changes such as changes in surface expression of non-targeted moiety cell-surface proteins are used as an indicator of a cell responses (phenotypic change). These changes may result in increased expression of proteins such as cytokine receptors, chemokine receptors, ion channels, transporters, adhesion receptors, immunological receptors (e.g. T-cell, B-cell, mast cell, macrophage, neutrophil, NK cell receptors) involved in stimulating or tampering immune responses, growth factor receptors, cell death receptors, photoreceptors, neural messenger receptors, receptors for cell differentiation, T-cell receptors, B-cell receptors, MHC I complexes, MHC II complexes, or receptors involved in tissue invasion, extravasation, phagocytosis, complement activation or recruitment, or senescence. Alternatively, stimulation with a targeting moiety may decrease the expression of the receptors described herein. Suitable antibodies include antibodies to a non-targeted receptor moiety that has an altered surface expression characteristic in response to a targeting moiety binding to the specific target moiety. Such antibodies may be used in conjunction with a fluorophore as a detection entity to measure the response of a particular cell in a particular microdrop relative to other cells in other microdrops in a plurality of microdrops (e.g. mammalian cell complexes). Labeling the microdrops with such an antibody and measuring the level of a phenotypic response enables the isolation of microdrops that has either increased surface expression of a receptor or decreased surface expression of a receptor relative to other cells in response to a targeting entity. Proteomic changes do not have to be limited to surface expression of receptors but may also include, e.g., soluble, secreted factors that are released in response to target entity stimulation with a targeting moiety. These factors include cytokines, chemokines, paracrine signaling molecules, autocrine signaling molecules, products of cell lysis and apoptosis, secondary messengers such as calcium or cAMP, and ions such as calcium, sodium, or potassium. The accumulation or reduction of these soluble molecules in the microdrop may be reported by a bead that is co-encapsulated with the secretory entity and the target entity that contains an antibody against the soluble agent. Measuring the levels of soluble molecules as collected by the bead within the microdrop through the use of an anti-molecule detection entity enables the isolation of microdrops that have increased or reduced levels of soluble molecules relative to other microdrops in the population and consequently targeting moieties that confer the phenotypic change can be isolated. The antibody against the soluble agent does not have to be localized to a bead but may be present on an additional entity (such as a cell) that may or not be the same cell as the secretory or target entities. The presence of the soluble molecule may then be reported by phenotypic changes in the additional cell. Such a system is suitable for selection of targeting moieties that are capable of perturbing paracrine or cytokine signaling. The antibody may alternatively be attached directly to the limited permeability matrix. For example, a biotinylated antibody is seeded in a matrix of biotinylated agarose by using strept-avidin to bridge the antibody and the agarose using methods that are well described in the art.

Phenotypic changes may be detected by changes in the transcriptome. Frequently, binding to a receptor causes changes in gene expression in the cell that has been bound. Methods are available to measure activation of genes and the transcription factors that govern their activation through the use of reporter genes. These reporter genes include genes such as GFP, YFP, BFP, RFP, beta-lactamase, beta-galactosidase, chloramphenicol acetyltransferase, neomycin phosphotransferase, and genes necessary for the production of essential metabolites like tryptophan, leucine, uracil, histidine, and methionine. These reporter genes are usually recombinantly expressed in such a way that they are under the control of a transcription promoter element that is itself under the control of a transcription factor that is responsive to the activation or deactivation of a receptor on the surface. Typically, the reporter gene is silenced unless transcription is activated by a transcription factor, but that does not always have to be the case. For example, a reporter gene such as the gene for GFP may be put under the control of a p53 response element. Stimulation of a cell receptor (the target moiety) by a targeting moiety that stimulates p53 transcriptional activity may be measured by the transcription and subsequent translation of GFP. Because GFP is fluorescent, the cell and thus the microdrop or mammalian cell complex is fluorescent which enables the isolation of microdrops that contain targeting moieties that stimulate the p53 pathway. Alternatively, selecting for microdrops that are not fluorescent under circumstances where they ordinarily would be (e.g. treating a plurality of complexes with a p53 agonist and then looking for targeting entities that block activation as measured by low GFP fluorescence) may also be used to discover targeting moieties. Suitable transcription factors and related pathways include but are not limited to c-Fos, c-Jun, NFκB, SP1, AP-1, C/EBP, Heat shock factor, ATF/CREB, c-myc, Oct-1, NF-1, MECP2, HNF, IPF1, FOXP2, FOXP3, p53, STAT, and HOX. Often transcription factors can be operably linked to other activation response elements such as kinases, inhibitors, and arrestins. For example, Life Technologies' TANGO assay relies on the fusion of arrestin with a protease that cleaves a transcription factor that is recombinantly fused to an expressed GPCR on the cell surface via a protease site. Stimulation of the GPCR by a binding moiety in such a way as to recruit arrestin also stimulates the cleavage of the transcription factor from the GPCR. The liberated transcription factor then stimulates the production of a reporter protein such as GFP or β-lactamase.

Other phenotypic changes suitable for detection include changes in the epigenome of the cell. These changes may reveal themselves as DNA methylation, chromatin remodeling, histone acetylation, methylation, ubiquitylation, phosphorylation, sumoylation, ribosylation, and citrullination that can be detected. Differential splicing of mRNA, silencing of translation of mRNA, expression of microRNA and sRNA, and protein modifications such as proteolysis, phosphorylation, ubiquitylation, sulfation, biotinylation, methylation, and glycosylation may also be indicators of phenotypic changes that are detectable. These changes may be measured by using a detection moiety that targets a specific epigenetic regulator such as a fluorophore-tagged anti-microRNA, fluorophore-tagged anti-histone deacetylase, or fluorophore-tagged methyl-DNA specific enzyme.

Other phenotypic changes suitable for detection include changes in the metabolic state of the cell or the metabolome. Reporters suitable to detect metabolic changes may include detection of the ability to utilize a particular carbon source in the presence of a targeting entity. Other reporters may detect the ability to metabolize a toxin or drug, the ability to process a substrate, the ability to import or export amino acids and ions, as well as the growth, senescence, or death of the cell. The phenotypic changes brought about by changes in the metabolome may also be detected by changes in cell polarization, voltage across the membrane, secondary messenger activity such as cAMP and calcium, cell size, cell viability, and the creation or elimination of small molecules (some of which would be naturally fluorescent such as FAD) in the cytosol of the target entity.

Pathways that lead to phenotypic changes and that are modulated through stimulation by a targeting moiety may include but are not limited to cAMP pathways, cADP-ribose and NAADP signaling, voltage-gated ion channels, receptor operated channels, PIP₂, PtdINS 3-kinase, nitric oxide/cGMP, redox signaling, MAPK, NFκB, phospholipase D, sphingomyelin, JAK/STAT, Smad, Wnt, Hedgehog, Hippo, Notch, ER stress signaling, and AMP signaling.

Aspects of the invention relate to microdrop and mammalian cell complex compositions and methods of selecting the same that are particularly suitable for the screening and identification of targeting moieties, such as antibodies, that are specific for (or have a high affinity for) target moieties selected from the group of cell membrane associated polypeptides, such as, e.g., ion channel proteins, transporter proteins, and G protein coupled receptors (GPCR). The target entities may display the membrane-associated polypeptides as functional fragments. In some embodiments, the membrane-associated polypeptides or subunits are displayed by the target entities as full length and are not functional fragments.

“G protein coupled receptors (GPCR)” include 5-Hydroxytryptamine receptors, Acetylcholine receptors (muscarinic), Adenosine receptors, Adrenoceptors, Angiotensin receptors, Apelin receptor, Bile acid receptor, Bombesin receptors, Bradykinin receptors, Cannabinoid receptors, Chemerin receptor, Chemokine receptors, Cholecystokinin receptors, Complement peptide receptors, Dopamine receptors, Endothelin receptors, Estrogen (G protein-coupled) receptor, Formylpeptide receptors, Free fatty acid receptors, Galanin receptors, Ghrelin receptor, Glycoprotein hormone receptors, Gonadotrophin-releasing hormone receptors, Histamine receptors, Hydroxycarboxylic acid receptors, Kisspeptin receptor, Leukotriene receptors, Lysophospholipid (LPA) receptors, Lysophospholipid (S1P) receptors, Melanin-concentrating hormone receptors, Melanocortin receptors, Melatonin receptors, Motilin receptor, Neuromedin U receptors, Neuropeptide FF/neuropeptide AF receptors, Neuropeptide S receptor, Neuropeptide W/neuropeptide B receptors, Neuropeptide Y receptors, Neurotensin receptors, Opioid receptors, Orexin receptors, Oxoglutarate receptor, P2Y receptors, Peptide P518 receptor, Platelet-activating factor receptor, Prokineticin receptors, Prolactin-releasing peptide receptor, Prostanoid receptors, Proteinase-activated receptors, Relaxin family peptide receptors, Somatostatin receptors, Succinate receptor, Tachykinin receptors, Thyrotropin-releasing hormone receptors, Trace amine receptor, Urotensin receptor, Vasopressin and oxytocin receptors, and Class A Orphans.

“Ion channels” include Voltage-gated ion channels, CatSper and Two-Pore channels, Cyclic nucleotide-regulated channels, Potassium channels, Calcium-activated potassium channels, Inwardly rectifying potassium channels, Two-P potassium channels, Voltage-gated potassium channels, Transient Receptor Potential channels, Voltage-gated calcium channels, Voltage-gated sodium channels, Ligand-gated ion channels, 5-HT3 receptors, GABAA receptors, Glycine receptors, Ionotropic glutamate receptors, Nicotinic acetylcholine receptors, P2X receptors, and Zink-activated ion channel (ZAC).

“Transporters” include pores and channels, such as alpha-helical channels, and beta-strand porins; electrochemical-potential-driven transporters, such as, uniporters, symporters and antiporters; primary active transporters, such as P—P-bond-hydrolysis-driven transporters (e.g. ATP-binding-cassette superfamily, ABC-type exporters), decarboxylation-driven transporters (e.g. Na⁺-transporting carboxylic acid decarboxylase), methyl-transfer-driven transporters (e.g. Na+-transporting methyltetrahydromethanopterin-CoM methyltransferase), oxidoreduction-driven transporters (e.g. proton (H⁺ or Na⁺)-translocating NADH dehydrogenases), light-driven transporters; phosphotransferases; and transmembrane electron carriers.

In certain embodiments, suitable G protein coupled receptors (GPCR), ion channel proteins and transporter proteins for the methods and microdrop compositions described herein include HTR1A, HTR1B, HTR1D, HTR1E, HTR1F, HTR2A, HTR2B, HTR2C, HTR4, HTR5A, HTR5BP, HTR6, HTR7, CHRM1, CHRM2, CHRM3, CHRM4, CHRM5, ADORA1, ADORA2A, ADORA2B, ADORA3, BAI1, BAI2, BAI3, CD97, CELSR1, CELSR2, CELSR3, ELTD1, EMR1, EMR2, EMR3, EMR4P, GPR56, GPR64, GPR97, GPR98, GPR110, GPR111, GPR112, GPR113, GPR114, GPR115, GPR116, GPR123, GPR124, GPR125, GPR126, GPR128, GPR133, GPR144, LPHN1, LPHN2, LPHN3, ADRA1A, ADRA1B, ADRA1D, ADRA2A, ADRA2B, ADRA2C, ADRB1, ADRB2, ADRB3, AGTR1, AGTR2, APLNR, GPBAR1, NMBR, BRS3, GRPR, BDKRB1, BDKRB2, CALCR, CALCRL, CASR, GPRC6A, CNR1, CNR2, CMKLR1, CCR1, CCR2, CCR3, CCR4, CCR5, CCR6, CCR7, CCR8, CCR9, CCR10, CXCR1, CXCR2, CXCR3, CXCR4, CXCR5, CXCR6, CX3CR1, XCR1, DARC, ACKR2, ACKR3, ACKR4, CCRL2, CCKAR, CCKBR, C3AR1, C5AR1, C5AR2, CRHR1, CRHR2, DRD1, DRD2, DRD3, DRD4, DRD5, EDNRA, EDNRB, GPER1, FPR1, FPR2, FPR3, FFAR1, FFAR2, FFAR3, FFAR4, GPR42, FZD1, FZD2, FZD3, FZD4, FZD5, FZD6, FZD7, FZD8, FZD9, FZD10, SMO, GABBR1, GABBR2, GALR1, GALR2, GALR3, GHSR, GHRHR, GIPR, GLP1R, GLP2R, GCGR, SCTR, FSHR, LHCGR, TSHR, GNRHR, GNRHR2, GPR18, GPR55, GPR119, HRH1, HRH2, HRH3, HRH4, HCAR1, HCAR2, HCAR3, KISS1R, LTB4R, LTB4R2, CYSLTR1, CYSLTR2, OXER1, FPR2, LPAR1, LPAR2, LPAR3, LPAR4, LPAR5, LPAR6, S1PR1, S1PR2, S1PR3, S1PR4, S1PR5, MCHR1, MCHR2, MC1R, MC2R, MC3R, MC4R, MC5R, MTNR1A, MTNR1B, GRM1, GRM2, GRM3, GRM4, GRM5, GRM6, GRM7, GRM8, MLNR, NMUR1, NMUR2, NPFFR1, NPFFR2, NPSR1, NPBWR1, NPBWR2, NPY1R, NPY2R, NPY4R, NPY5R, NPY6R, NTSR1, NTSR2, OPRD1, OPRK1, OPRM1, OPRL1, HCRTR1, HCRTR2, OXGR1, P2RY1, P2RY2, P2RY4, P2RY6, P2RY11, P2RY12, P2RY13, P2RY14, PTH1R, PTH2R, QRFPR, PTAFR, PROKR1, PROKR2, PRLHR, PTGDR, PTGDR2, PTGER1, PTGER2, PTGER3, PTGER4, PTGFR, PTGIR, TBXA2R, F2R, F2RL1, F2RL2, F2RL3, RXFP1, RXFP2, RXFP3, RXFP4, SSTR1, SSTR2, SSTR3, SSTR4, SSTR5, SUCNR1, TACR1, TACR2, TACR3, TRHR, TAAR1, UTS2R, AVPR1A, AVPR1B, AVPR2, OXTR, ADCYAP1R1, VIPR1, VIPR2, BRS3, GPR1, GPR3, GPR4, GPR6, GPR12, GPR15, GPR17, GPR18, GPR19, GPR20, GPR21, GPR22, GPR25, GPR26, GPR27, GPR31, GPR32, GPR33, GPR34, GPR35, GPR37, GPR37L1, GPR39, GPR42, GPR45, GPR50, GPR52, GPR55, GPR61, GPR62, GPR63, GPR65, GPR68, GPR75, GPR78, GPR79, GPR82, GPR83, GPR84, GPR85, GPR87, GPR88, GPR101, GPR119, GPR132, GPR135, GPR139, GPR141, GPR142, GPR146, GPR148, GPR149, GPR150, GPR151, GPR152, GPR153, GPR160, GPR161, GPR162, GPR171, GPR173, GPR174, GPR176, GPR182, GPR183, LGR4, LGR5, LGR6, MAS1, MAS1L, MRGPRD, MRGPRE, MRGPRF, MRGPRG, MRGPRX1, MRGPRX2, MRGPRX3, MRGPRX4, OPN3, OPN4, OPN5, P2RY8, P2RY10, TAAR2, TAAR3, TAAR4P, TAAR5, TAAR6, TAAR8, TAAR9, GPR156, GPR158, GPR179, GPRC5A, GPRC5B, GPRC5C, GPRC5D, TAS1R1, TAS1R2, TAS1R3, TAS2R1, TAS2R3, TAS2R4, TAS2R5, TAS2R7, TAS2R8, TAS2R9, TAS2R10, TAS2R13, TAS2R14, TAS2R16, TAS2R19, TAS2R20, TAS2R42, TAS2R30, TAS2R31, TAS2R39, TAS2R40, TAS2R50, TAS2R43, TAS2R46, TAS2R41, TAS2R60, TAS2R38, GPR107, GPR137, OR51E1, TPRA1, GPR143, GPR157, THRA, THRB, RARA, RARB, RARG, PPARA, PPARD, PPARG, NR1D1, NR1D2, RORA, RORB, RORC, NR1H4, NR1H5P, NR1H3, NR1H2, VDR, NR1I2, NR1I3, HNF4A, HNF4G, RXRA, RXRB, RXRG, NR2C1, NR2C2, NR2E1, NR2E3, NR2F1, NR2F2, NR2F6, ESR1, ESR2, ESRRA, ESRRB, ESRRG, AR, NR3C1, NR3C2, PGR, NR4A1, NR4A2, NR4A3, NR5A1, NR5A2, NR6A1, NR0B1, NR0B2, KCNMA1, KCNN1, KCNN2, KCNN3, KCNN4, KCNT1, KCNT2, KCNU1, CATSPER1, CATSPER2, CATSPER3, CATSPER4, TPCN1, TPCN2, CNGA1, CNGA2, CNGA3, CNGA4, CNGB1, CNGB3, HCN1, HCN2, HCN3, HCN4, KCNJ1, KCNJ2, KCNJ12, KCNJ4, KCNJ14, KCNJ3, KCNJ6, KCNJ9, KCNJ5, KCNJ10, KCNJ15, KCNJ16, KCNJ8, KCNJ11, KCNJ13, TRPA1, TRPC1, TRPC2, TRPC3, TRPC4, TRPC5, TRPC6, TRPC7, TRPM1, TRPM2, TRPM3, TRPM4, TRPM5, TRPM6, TRPM7, TRPM8, MCOLN1, MCOLN2, MCOLN3, PKD2, PKD2L1, PKD2L2, TRPV1, TRPV2, TRPV3, TRPV4, TRPV5, TRPV6, KCNK1, KCNK2, KCNK3, KCNK4, KCNK5, KCNK6, KCNK7, KCNK9, KCNK10, KCNK12, KCNK13, KCNK15, KCNK16, KCNK17, KCNK18, CACNA1S, CACNA1C, CACNA1D, CACNA1F, CACNA1A, CACNA1B, CACNA1E, CACNA1G, CACNA1H, CACNA1I, KCNA1, KCNA2, KCNA3, KCNA4, KCNA5, KCNA6, KCNA7, KCNA10, KCNB1, KCNB2, KCNC1, KCNC2, KCNC3, KCNC4, KCND1, KCND2, KCND3, KCNF1, KCNG1, KCNG2, KCNG3, KCNG4, KCNQ1, KCNQ2, KCNQ3, KCNQ4, KCNQ5, KCNV1, KCNV2, KCNS1, KCNS2, KCNS3, KCNH1, KCNH5, KCNH2, KCNH6, KCNH7, KCNH8, KCNH3, KCNH4, HVCN1, SCN1A, SCN2A, SCN3A, SCN4A, SCN5A, SCN8A, SCN9A, SCN10A, SCN11A, HTR3A, HTR3B, HTR3C, HTR3D, HTR3E, ASIC1, ASIC2, ASIC3, SCNN1A, SCNN1B, SCNN1D, SCNN1G, GABRA1, GABRA2, GABRA3, GABRA4, GABRA5, GABRA6, GABRB1, GABRB2, GABRB3, GABRG1, GABRG2, GABRG3, GABRD, GABRE, GABRQ, GABRP, GABRR1, GABRR2, GABRR3, GLRA1, GLRA2, GLRA3, GLRA4, GLRB, GRIA1, GRIA2, GRIA3, GRIA4, GRID1, GRID2, GRIK1, GRIK2, GRIK3, GRIK4, GRIK5, GRIN1, GRIN2A, GRIN2B, GRIN2C, GRIN2D, GRIN3A, GRIN3B, ITPR1, ITPR2, ITPR3, CHRNA1, CHRNA2, CHRNA3, CHRNA4, CHRNA5, CHRNA6, CHRNA7, CHRNA9, CHRNA10, CHRNB1, CHRNB2, CHRNB3, CHRNB4, CHRNG, CHRND, CHRNE, P2RX1, P2RX2, P2RX3, P2RX4, P2RX5, P2RX6, P2RX7, RYR1, RYR2, RYR3, ZACN, CLCN1, CLCN2, CLCNKA, CLCNKB, CLCN3, CLCN4, CLCN5, CLCN6, CLCN7, CFTR, ANO1, MIP, AQP1, AQP2, AQP3, AQP4, AQP5, AQP6, AQP7, AQP8, AQP9, AQP10, GJE1, GJB7, GJB2, GJB6, GJC3, GJB4, GJB3, GJB5, GJD3, GJB1, GJD2, GJA4, GJA5, GJD4, GJA1, GJC1, GJA3, GJC2, GJA8, GJA9, GJA10, PANX1, PANX2, PANX3, NALCN, GFRA1, GFRA2, GFRA3, GFRA4, ITGA1, ITGA2, ITGA2B, ITGA3, ITGA4, ITGA5, ITGA6, ITGA7, ITGA8, ITGA9, ITGA10, ITGA11, ITGAD, ITGAE, ITGAL, ITGAM, ITGAV, ITGAX, ITGB1, ITGB2, ITGB3, ITGB4, ITGB5, ITGB6, ITGB7, ITGB8, GUCY2C, NPR1, NPR2, NPR3, PTPRA, PTPRB, PTPRC, PTPRD, PTPRE, PTPRF, PTPRG, PTPRH, PTPRJ, PTPRK, PTPRM, PTPRN, PTPRN2, PTPRO, PTPRQ, PTPRR, PTPRS, PTPRT, PTPRU, PTPRZ1, TNFRSF1A, TNFRSF1B, LTBR, TNFRSF4, CD40, FAS, TNFRSF6B, CD27, TNFRSF8, TNFRSF9, TNFRSF10A, TNFRSF10B, TNFRSF10C, TNFRSF10D, TNFRSF11A, TNFRSF11B, TNFRSF25, TNFRSF12A, TNFRSF13B, TNFRSF13C, TNFRSF14, NGFR, TNFRSF17, TNFRSF18, TNFRSF19, RELT, TNFRSF21, EDA2R, EDAR, IL13RA2, IL2RA, IL2RB, IL2RG, IL4R, IL7R, IL9R, IL13RA1, IL15RA, IL21R, CRLF2, IL3RA, IL5RA, CSF2RA, CSF2RB, LEPR, IL6R, IL6ST, IL11RA, IL27RA, IL31RA, CNTFR, LIFR, OSMR, IL12RB1, IL12RB2, IL23R, EPOR, CSF3R, GHR, PRLR, MPL, IFNAR1, IFNAR2, IFNGR1, IFNGR2, IL22RA2, IL10RA, IL10RB, IL20RA, IL20RB, IL22RA1, IFNLR1, IL1R1, IL1R2, IL1RL1, IL1RL2, IL18R1, IL17RA, IL17RB, IL17RC, IL17RD, IL17RE, TLR1, TLR2, TLR3, TLR4, TLR5, TLR6, TLR7, TLR8, TLR9, TLR10, NOD1, NOD2, NLRC3, NLRC5, NLRX1, CIITA, NLRP1, NLRP2, NLRP3, NLRP4, NLRP5, NLRP6, NLRP7, NLRP8, NLRP9, NLRP10, NLRP11, NLRP12, NLRP13, NLRP14, NLRC4, NAIP, ACVRL1, ACVR1, BMPR1A, ACVR1B, TGFBR1, BMPR1B, ACVR1C, ACVR2A, ACVR2B, AMHR2, BMPR2, TGFBR2, TGFBR3, EGFR, ERBB2, ERBB3, ERBB4, INSR, IGF1R, INSRR, PDGFRA, PDGFRB, KIT, CSF1R, FLT3, FLT1, KDR, FLT4, FGFR1, FGFR2, FGFR3, FGFR4, PTK7, NTRK1, NTRK2, NTRK3, ROR1, ROR2, MUSK, MET, MST1R, AXL, TYRO3, MERTK, TIE1, TEK, EPHA1, EPHA2, EPHA3, EPHA4, EPHA5, EPHA6, EPHA7, EPHA8, EPHA10, EPHB1, EPHB2, EPHB3, EPHB4, EPHB6, RET, RYK, DDR1, DDR2, ROS1, AATK, LMTK2, LMTK3, LTK, ALK, STYK1, GUCY2C, GUCY2D, GALNS, BCR, KDM1A, KDM1B, KDM2A, KDM2B, KDM3A, KDM3B, KDM4A, KDM4B, KDM4C, KDM4D, KDM4E, KDM5A, KDM5B, KDM5C, KDM5D, KDM6A, KDM6B, KDM7A, KDM8, PHF2, PHF8, ASH1L, DOT1L, EHMT1, EHMT2, EZH2, KMT2A, KMT2B, KMT2C, KMT2D, KMT2E, NSD1, PRDM2, SETD1A, SETD1B, SETD2, SETD7, SETD8, SETDB1, SETDB2, SMYD2, SUV39H1, SUV39H2, SUV420H1, SUV420H2, CLOCK, ELP3, GTF3C4, HAT1, JMJD1C, KAT5, KAT6A, KAT6B, KAT7, KAT8, NCOA1, NCOA2, NCOA3, KAT2A, KAT2B, ATAD2, ATAD2B, CHAT, ACHE, BCHE, ADA, ADK, NT5E, AHCY, NT5C1A, NT5C1B, NT5C2, NT5C3A, NT5C, NT5M, PAH, TH, TPH1, TPH2, PRMT1, PRMT2, PRMT3, CARM1, PRMT5, PRMT6, PRMT7, PRMT8, FBXO11, PRMT10, FBXO10, ARG1, ARG2, GATM, DDAH1, DDAH2, NOS3, NOS2, NOS1, PC, ACACA, ACACB, PCCA, PCCB, GGCX, AMD1, GAD1, GAD2, ADC, DDC, HDC, MLYCD, ODC1, PISD, PAH, TAT, DDC, TH, DBH, PNMT, MAOA, MAOB, COMT, SPTLC1, SPTLC2, SPTLC3, SPTSSA, SPTSSB, KDSR, CERS1, CERS2, CERS3, CERS4, CERS5, CERS6, DEGS1, DEGS2, SGMS1, SGMS2, SAMD8, SMPD1, SMPD2, SMPD3, SMPD4, SMPDL3A, SMPDL3B, EED, NSMAF, UGCG, ASAH1, ASAH2, ASAH2B, ASAH2C, ACER1, ACER2, ACER3, CERK, ADCY1, ADCY2, ADCY3, ADCY4, ADCY5, ADCY6, ADCY7, ADCY8, ADCY9, GUCY1A3, GUCY1A2, GUCY1B3, GUCY1B2, RAPGEF3, RAPGEF4, PDE1A, PDE1B, PDE1C, PDE2A, PDE3A, PDE3B, PDE4A, PDE4B, PDE4C, PDE4D, PDE5A, PDE6A, PDE6B, PDE6C, PDE6D, PDE6G, PDE6H, PDE7A, PDE7B, PDE8A, PDE8B, PDE9A, PDE10A, PDE11A, CYP1A1, CYP1A2, CYP1B1, CYP2A6, CYP2A7, CYP2A13, CYP2B6, CYP2C8, CYP2C9, CYP2C18, CYP2C19, CYP2D6, CYP2E1, CYP2F1, CYP2J2, CYP2R1, CYP2S1, CYP2U1, CYP2W1, CYP3A4, CYP3A5, CYP3A7, CYP3A43, CYP4A11, CYP4A22, CYP4B1, CYP4F2, CYP4F3, CYP4F8, CYP4F11, CYP4F12, CYP4F22, CYP4V2, CYP4X1, CYP4Z1, TBXAS1, PTGIS, CYP7A1, CYP7B1, CYP8B1, CYP11A1, CYP11B1, CYP11B2, CYP17A1, CYP19A1, CYP20A1, CYP21A2, CYP24A1, CYP26A1, CYP26B1, CYP26C1, CYP27A1, CYP27B1, CYP27C1, CYP39A1, CYP46A1, CYP51A1, DAGLA, DAGLB, NAPEPLD, MGLL, FAAH, FAAH2, NAAA, PTGS1, PTGS2, TBXAS1, PTGIS, PTGES, PTGES2, PTGES3, PTGDS, HPGDS, AKR1C3, CBR1, HPGD, ALOX5, ALOX12B, ALOX12, ALOX15, ALOX15B, ALOXE3, LTC4S, GGCT, DPEP1, DPEP2, LTA4H, GAD1, GAD2, ALDH9A1, ABAT, ALDH5A1, PLCB1, PLCB2, PLCB3, PLCB4, PLCG1, PLCG2, PLCD1, PLCD3, PLCD4, PLCE1, PLCZ1, PLCH1, PLCH2, PLA2G1B, PLA2G2A, PLA2G2D, PLA2G2E, PLA2G2F, PLA2G3, PLA2G10, PLA2G12A, PLA2G4A, PLA2G4B, PLA2G4C, PLA2G4D, PLA2G4E, PLA2G4F, PLA2G5, PLA2G6, PLA2G7, PAFAH2, PLD1, PLD2, LPIN1, LPIN2, LPIN3, PPAP2A, PPAP2B, PPAP2C, PTEN, PI4KA, PI4KB, PI4K2A, PI4K2B, PIK3CA, PIK3CB, PIK3CD, PIK3CG, PIK3R1, PIK3R2, PIK3R3, PIK3R4, PIK3R5, PIK3R6, PIK3C2A, PIK3C2B, PIK3C2G, PIK3C3, PIP5K1A, PIP5K1B, PIP5K1C, PIP4K2A, PIP4K2B, PIP4K2C, HMOX1, HMOX2, CBS, CTH, CCBL1, MPST, DAGLA, DAGLB, MGLL, FAAH, PLA2G2A, PLA2G7, PLD2, ACHE, LTA4H, BCHE, PNLIP, LIPG, CES1, LIPE, ITPKA, ITPKB, ITPKC, INPP1, INPP4A, INPP4B, INPP5A, INPP5B, INPP5D, INPP5E, INPP5J, INPP5K, INPPL1, OCRL, SYNJ1, SYNJ2, IMPA1, IMPA2, ACAT1, ACAT2, HMGCS1, HMGCS2, HMGCR, MVK, PMVK, MVD, IDI1, IDI2, GGPS1, FDPS, FDFT1, SQLE, LSS, SPHK1, SPHK2, SGPP1, SGPP2, SGPL1, TPO, DIO1, DIO2, DIO3, IYD, NIM1, ADCK2, ADCK1, ADCK3, ADCK4, ADCK5, TWF1, TWF2, TRPM6, TRPM7, EEF2K, CAMKK1, CAMKK2, PRKAA1, PRKAA2, PRKAB1, PRKAB2, PRKAG1, PRKAG2, PRKAG3, BRSK1, BRSK2, CHEK1, HUNK, STK11, MARK1, MARK2, MARK3, MARK4, MELK, NUAK1, NUAK2, PASK, SIK1, SIK2, SIK3, SNRK, CDK20, CDK4, CDK6, CDK9, CDK1, CDK2, CDK3, CDK10, CDK5, CDK7, CDK8, CDK19, CDK12, CDK13, CDK11A, CDK11B, CDK14, CDK15, CDK16, CDK17, CDK18, DMPK, CDC42BPG, CDC42BPA, CDC42BPB, DYRK1A, DYRK1B, DYRK4, DYRK2, DYRK3, HIPK1, HIPK2, HIPK3, HIPK4, PRPF4B, ADRBK1, ADRBK2, GRK1, GRK4, GRK5, GRK6, GRK7, GSK3A, GSK3B, LIMK1, LIMK2, TESK1, TESK2, MAPKAPK2, MAPKAPK3, MAPKAPK5, MKNK1, MKNK2, MAPK1, MAPK3, MAPK4, MAPK6, MAPK7, MAPK15, MAPK8, MAPK9, MAPK10, MAPK11, MAPK12, MAPK13, MAPK14, NLK, TNNI3K, ILK, MAP3K12, MAP3K13, MAP3K9, MAP3K10, MAP3K11, ZAK, MAP3K7, EIF2AK4, EIF2AK3, ATR, MTOR, SMG1, TRRAP, PRKCB, PRKCG, PRKCA, PRKCD, PRKCQ, PRKCE, PRKCH, PRKCI, PRKCZ, RIOK1, RIOK2, RIOK3, RPS6KA5, RPS6KA4, RPS6KB1, RPS6KB2, RPS6KA1, RPS6KA3, RPS6KA2, RPS6KA6, OXSR1, STK39, SGK494, MAP4K1, MAP4K2, MAP4K3, MAP4K5, MAP4K4, MINK1, NRK, TNIK, STK3, STK4, MYO3A, MYO3B, PAK1, PAK2, PAK3, PAK4, PAK6, PAK7, SLK, STK10, STRADA, STRADB, TAOK1, TAOK2, TAOK3, STK24, STK25, MST4, ABL1, ABL2, TNK1, TNK2, ALPK1, ALPK3, AURKA, AURKB, AURKC, BRD1, BRD2, BRD3, BRD4, BRD7, BRD8, BRD9, BUB1, BUB1B, TP53RK, CAMK1, CAMK1D, CAMK1G, CAMK4, PNCK, CAMK2A, CAMK2B, CAMK2G, CAMK2D, CAMKV, STK33, STK40, CSNK1A1, CSNK1A1L, CSNK1G1, CSNK1G2, CSNK1G3, CSNK1D, CSNK1E, CSNK2A1, CSNK2A2, CSNK2B, CASK, CDC7, CLK1, CLK2, CLK3, CLK4, CSK, MATK, CDKL1, CDKL2, CDKL3, CDKL4, CDKL5, DCLK1, DCLK2, DCLK3, DAPK1, DAPK2, DAPK3, STK17A, STK17B, CIT, PTK2, PTK2B, FER, FES, STK19, GSG2, CHUK, IKBKB, IKBKE, TBK1, IRAK1, IRAK2, IRAK3, IRAK4, ERN1, ERN2, JAK1, JAK2, JAK3, TYK2, LRRK1, LRRK2, MAST1, MAST2, MAST3, MAST4, MASTL, MOS, MYLK, MYLK2, MYLK3, MYLK4, TTN, AAK1, STK16, LATS1, LATS2, STK38, STK38L, NEK1, NEK2, NEK3, NEK4, NEK5, NEK6, NEK7, NEK8, NEK9, NEK10, NEK11, SBK1, SBK2, SGK110, PINK1, PDIK1L, STK35, TEX14, NRBP1, NRBP2, BMP2K, GAK, C9orf96, DSTYK, STK31, UHMK1, PDK2, PDK3, PDK4, EIF2AK1, EIF2AK2, ATM, PHKG1, PHKG2, PIM1, PIM2, PIM3, PLK1, PLK2, PLK3, PLK4, PDPK1, PRKAR1A, PRKAR1B, PRKAR2A, PRKAR2B, PRKACA, PRKACB, PRKACG, PRKX, PRKY, AKT1, AKT2, AKT3, PRKD1, PRKD2, PRKD3, PRKG1, PRKG2, ROCK1, ROCK2, PKN1, PKN2, PKN3, PSKH1, PSKH2, BCKDK, CHEK2, ARAF, BRAF, KSR1, KSR2, RAF1, ICK, MAK, MOK, ANKK1, RIPK1, RIPK2, RIPK3, RIPK4, RPS6KC1, RPS6KL1, SCYL1, SCYL2, SGK1, SGK2, SGK3, PKDCC, PXK, BLK, FGR, FRK, FYN, HCK, LCK, LYN, PTK6, SRC, SRMS, YES1, SRPK1, SRPK2, SRPK3, MAP3K1, MAP3K2, MAP3K3, MAP3K4, MAP3K5, MAP3K6, MAP3K15, MAP3K19, MAP2K1, MAP2K2, MAP2K3, MAP2K4, MAP2K5, MAP2K6, MAP2K7, MAP3K14, MAP3K8, SYK, ZAP70, TAF1, TAF1L, TTBK1, TTBK2, TBCK, BMX, BTK, ITK, TEC, TXK, TSSK1B, TSSK2, TSSK3, TSSK4, TSSK6, TRIM24, TRIM28, TRIM33, MLKL, PBK, TLK1, TLK2, TRIB1, TRIB2, TRIB3, KALRN, OBSCN, SPEG, TRIO, TTK, PI4KA, PI4KB, STK36, ULK1, ULK2, ULK3, ULK4, VRK1, VRK2, VRK3, PIK3R4, PKMYT1, WEE1, WEE2, WNK1, WNK2, WNK3, WNK4, STK32A, STK32B, STK32C, PIK3C2A, PIK3C2B, PIK3C2G, PIK3C3, PIK3CA, PIK3CB, PIK3CD, PIK3CG, PIP4K2B, PIP4K2C, PIP5K1A, PIP5K1C, SPHK1, SPHK2, BACE1, BACE2, CTSD, CTSE, PGA5, PGC, REN, PSEN1, PSEN2, CTSB, CTSC, CTSF, CTSH, CTSK, CTSL, CTSV, CTSS, CTSZ, CAPN1, CAPN2, BAP1, UCHL1, UCHL3, LGMN, CASP1, CASP2, CASP3, CASP4, CASP5, CASP6, CASP7, CASP8, CASP9, CASP10, CASP14, USP1, USP2, USP5, USP14, GGH, PPAT, SENP1, SENP6, SENP7, SENP8, ATG4B, ANPEP, C9orf3, RNPEP, RNPEPL1, ERAP1, ERAP2, ENPEP, LNPEP, LTA4H, NPEPPS, TRHDE, ACE, ACE2, MMP1, MMP2, MMP3, MMP7, MMP8, MMP9, MMP10, MMP11, MMP12, MMP13, MMP14, MMP15, MMP16, MMP17, MMP19, MMP20, MMP21, MMP23B, MMP24, MMP25, MMP26, MMP27, MMP28, ADAM2, ADAM7, ADAM8, ADAM9, ADAM10, ADAM11, ADAM12, ADAM15, ADAM17, ADAM18, ADAM19, ADAM20, ADAM21, ADAM22, ADAM23, ADAM28, ADAM29, ADAM30, ADAM32, ADAM33, ADAMTS1, ADAMTS2, ADAMTS3, ADAMTS4, ADAMTS5, ADAMTS6, ADAMTS7, ADAMTS8, ADAMTS9, ADAMTS10, ADAMTS12, ADAMTS13, ADAMTS14, ADAMTS15, ADAMTS16, ADAMTS17, ADAMTS18, ADAMTS19, ADAMTS20, BMP1, ECE1, ECE2, MME, AEBP1, CPA1, CPA2, CPA3, CPA4, CPA5, CPA6, CPB1, CPB2, CPD, CPE, CPM, CPN1, CPN2, CPO, CPQ, CPXM1, CPXM2, CPZ, IDE, NPEPL1, LAP3, DNPEP, DPEP1, CNDP1, CNDP2, METAP1, METAP2, METAP1D, PEPD, XPNPEP1, XPNPEP2, XPNPEP3, FOLH1B, FOLH1, QPCT, NAALADL1, NAALAD2, DPP3, PSMD14, RCE1, ACR, CTSG, CMA1, CTRC, CTRL, CELA1, C1R, C1S, CFB, F2, F7, F9, F10, F11, F12, ELANE, GZMA, GZMB, GZMK, KLKB1, KLK2, KLK3, KLK4, KLK5, KLK6, KLK7, KLK8, PLG, PLAT, PLAU, PRSS1, PRSS2, PRSS3, PRSS8, PROC, PRTN3, ST14, TMPRSS2, TMPRSS6, TMPRSS11D, TPSAB1, TPSG1, FURIN, MBTPS1, PCSK1, PCSK2, PCSK4, PCSK5, PCSK6, PCSK7, PCSK9, TPP2, APEH, DPP4, DPP8, DPP9, FAP, PREP, CTSA, SCPEP1, CPVL, DPP7, PRCP, PSMB1, PSMB2, PSMB5, PSMB6, PSMB8, PSMB9, TASP1, ALDH2, DHFR, DHODH, GSR, HPD, HSD3B2, IMPDH1, IMPDH2, SRD5A2, TYR, VKORC1, XDH, HSD11B1, AOC3, AKR1B1, RRM1, RRM2, RRM2B, TYMS, DNMT1, DNMT3A, GART, FASN, PARP1, PARP2, LIPF, ASPG, AMY2A, GAA, MGAM, HDAC1, HDAC2, HDAC3, HDAC4, HDAC5, HDAC6, HDAC7, HDAC8, HDAC9, HDAC10, HDAC11, SIRT1, SIRT2, SIRT3, SIRT4, SIRT5, SIRT6, SIRT7, CA1, CA4, CA7, CA12, CA13, CA14, HSD3B2, FKBP1A, PPIA, TOP1, TOP1MT, TOP2A, GART, ABCA1, ABCA2, ABCA3, ABCA4, ABCA5, ABCA6, ABCA7, ABCA8, ABCA9, ABCA10, ABCA12, ABCA13, ABCB1, TAP1, TAP2, ABCB4, ABCB5, ABCB6, ABCB7, ABCB8, ABCB9, ABCB10, ABCB11, ABCC1, ABCC2, ABCC3, ABCC4, ABCC5, ABCC6, ABCC8, ABCC10, ABCC11, ABCC12, ABCC9, SV2A, ABCD1, ABCD2, ABCD3, ABCG1, ABCG2, ABCG4, ABCG5, ABCG8, ATP5A1, ATP5B, ATP5C1, ATP5D, ATP5E, MT-ATP6, ATP5F1, ATP5G1|ATP5G2|ATP5G3, ATP5H, ATP5I, ATP5J2, ATP5J, ATP5L2, MT-ATP8, ATP6V1A, ATP6V1B1, ATP6V1B2, ATP6V1C1, ATP6V1C2, ATP6V1D, ATP6V1E1, ATP6V1E2, ATP6V1F, ATP6V1G1, ATP6V1G2, ATP6V1G3, ATP6V1H, ATP6V0A1, ATP6V0A2, TCIRG1, ATP6V0A4, ATP6V0B, ATP6V0C, ATP6V0D1, ATP6V0D2, ATP6V0E1, ATP6V0E2, ATP1A1, ATP1A2, ATP1A3, ATP1A4, ATP1B1, ATP1B2, ATP1B3, ATP2A1, ATP2A2, ATP2A3, ATP2B1, ATP2B2, ATP2B3, ATP2B4, ATP2C1, ATP2C2, ATP4A, ATP12A, ATP4B, ATP7A, ATP7B, ATP8A1, ATP8A2, ATP8B1, ATP8B2, ATP8B3, ATP8B4, ATP9A, ATP9B, ATP10A, ATP10B, ATP10D, ATP11A, ATP11B, ATP11C, SLC1A3, SLC1A2, SLC1A1, SLC1A6, SLC1A7, SLC1A4, SLC1A5, SLC2A1, SLC2A2, SLC2A3, SLC2A4, SLC2A14, SLC2A5, SLC2A7, SLC2A9, SLC2A11, SLC2A6, SLC2A8, SLC2A10, SLC2A12, SLC2A13, SLC3A1, SLC3A2, SLC7A1, SLC7A2, SLC7A3, SLC7A4, SLC7A14, SLC7A5, SLC7A8, SLC7A7, SLC7A6, SLC7A9, SLC7A10, SLC7A11, SLC7A13, SLC4A1, SLC4A2, SLC4A3, SLC4A9, SLC4A4, SLC4A5, SLC4A7, SLC4A10, SLC4A8, SLC4A11, SLC5A1, SLC5A2, SLC5A4, SLC5A9, SLC5A10, SLC5A7, SLC5A5, SLC5A6, SLC5A8, SLC5A12, SLC5A3, SLC5A11, SLC6A2, SLC6A3, SLC6A4, SLC6A1, SLC6A13, SLC6A11, SLC6A12, SLC6A6, SLC6A8, SLC6A9, SLC6A5, SLC6A14, SLC6A7, SLC6A19, SLC6A15, SLC6A18, SLC6A16, SLC6A17, SLC6A20, SLC8A1, SLC8A2, SLC8A3, SLC9A1, SLC9A2, SLC9A3, SLC9A4, SLC9A5, SLC9A6, SLC9A7, SLC9A8, SLC9A9, SLC9B1, SLC9B2, SLC9C1, SLC9C2, SLC10A1, SLC10A2, SLC10A3, SLC10A4, SLC10A5, SLC10A6, SLC10A7, SLC11A1, SLC11A2, SLC12A1, SLC12A2, SLC12A3, SLC12A4, SLC12A5, SLC12A6, SLC12A7, SLC12A8, SLC12A9, SLC13A1, SLC13A2, SLC13A3, SLC13A4, SLC13A5, SLC14A1, SLC14A2, SLC15A1, SLC15A2, SLC15A3, SLC15A4, SLC16A1, SLC16A7, SLC16A8, SLC16A3, SLC16A4, SLC16A5, SLC16A6, SLC16A2, SLC16A9, SLC16A10, SLC16A11, SLC16A12, SLC16A13, SLC16A14, SLC17A1, SLC17A2, SLC17A3, SLC17A4, SLC17A5, SLC17A7, SLC17A6, SLC17A8, SLC17A9, SLC18A1, SLC18A2, SLC18A3, SLC18B1, SLC19A1, SLC19A2, SLC19A3, SLC20A1, SLC20A2, SLC22A1, SLC22A2, SLC22A3, SLC22A4, SLC22A5, SLC22A16, SLC22A6, SLC22A7, SLC22A8, SLC22A9, SLC22A10, SLC22A11, SLC22A12, SLC22A13, SLC22A14, SLC22A15, SLC22A17, SLC22A18, SLC22A20, SLC22A23, SLC22A24, SLC22A25, SLC22A31, SLC23A1, SLC23A2, SLC23A3, SLC23A4P, SLC24A1, SLC24A2, SLC24A3, SLC24A4, SLC24A5, SLC8B1, SLC25A1, SLC25A10, SLC25A11, SLC25A21, SLC25A34, SLC25A35, SLC25A47, SLC25A48, SLC25A12, SLC25A13, SLC25A18, SLC25A22, SLC25A2, SLC25A15, SLC25A20, SLC25A29, SLC25A38, SLC25A39, SLC25A40, SLC25A44, SLC25A45, SLC25A3, SLC25A4, SLC25A5, SLC25A6, SLC25A31, SLC25A16, SLC25A17, SLC25A19, SLC25A26, SLC25A42, SLC25A24, SLC25A23, SLC25A25, SLC25A32, SLC25A33, SLC25A36, SLC25A41, SLC25A43, UCP1, UCP2, UCP3, SLC25A27, SLC25A14, SLC25A30, MTCH1, MTCH2, SLC25A51, SLC25A52, SLC25A53, SLC25A28, SLC25A37, SLC25A46, SLC26A1, SLC26A2, SLC26A3, SLC26A4, SLC26A6, SLC26A7, SLC26A9, SLC26A5, SLC26A8, SLC26A10, SLC26A11, SLC27A1, SLC27A2, SLC27A3, SLC27A4, SLC27A5, SLC27A6, SLC28A1, SLC28A2, SLC28A3, SLC29A1, SLC29A2, SLC29A3, SLC29A4, SLC30A1, SLC30A2, SLC30A3, SLC30A4, SLC30A5, SLC30A6, SLC30A7, SLC30A8, SLC30A9, SLC30A10, SLC31A1, SLC31A2, SLC32A1, SLC33A1, SLC34A1, SLC34A2, SLC34A3, SLC35A1, SLC35A2, SLC35A3, SLC35A4, SLC35A5, SLC35B1, SLC35B2, SLC35B3, SLC35B4, SLC35C1, SLC35C2, SLC35D1, SLC35D2, SLC35D3, SLC35E1, SLC35E2, SLC35E2B, SLC35E3, SLC35E4, SLC35F1, SLC35F2, SLC35F3, SLC35F4, SLC35F5, SLC35F6, SLC35G1, SLC35G2, SLC35G3, SLC35G4, SLC35G5, SLC35G6, SLC36A1, SLC36A2, SLC36A3, SLC36A4, SLC37A1, SLC37A2, SLC37A3, SLC37A4, SLC38A1, SLC38A2, SLC38A4, SLC38A3, SLC38A5, SLC38A6, SLC38A7, SLC38A8, SLC38A9, SLC38A10, SLC38A11, SLC39A1, SLC39A2, SLC39A3, SLC39A4, SLC39A5, SLC39A6, SLC39A7, SLC39A8, SLC39A9, SLC39A10, SLC39A11, SLC39A12, SLC39A13, SLC39A14, SLC40A1, SLC41A1, SLC41A2, SLC41A3, RHAG, RHBG, RHCG, SLC43A1, SLC43A2, SLC43A3, SLC44A1, SLC44A2, SLC44A3, SLC44A4, SLC44A5, SLC45A1, SLC45A2, SLC45A3, SLC45A4, SLC46A1, SLC46A2, SLC46A3, SLC47A1, SLC47A2, SLC48A1, FLVCR1, FLVCR2, MFSD7, DIRC2, SLC50A1, SLC51A, SLC51B, SLC52A1, SLC52A2, SLC52A3, SLCO1A2, SLCO1B1, SLCO1B3, SLCO1C1, SLCO2A1, SLCO2B1, SLCO3A1, SLCO4A1, SLCO4C1, SLCO5A1, SLCO6A1, EEF2, KEAP1, ADIPOR1, ADIPOR2, FABP1, FABP2, FABP3, FABP4, FABP5, FABP6, FABP7, PMP2, FABP9, FABP12, RBP1, RBP2, RBP3, RBP4, RBP5, RBP7, RLBP1, CRABP1, CRABP2, HSPA1A, HSPA1B, HSPA2, HSPA6, HSPA8, BAZ2A, BAZ2B, BPTF, BRDT, BRPF1, BRPF3, BRWD1, CECR2, CREBBP, EP300, PBRM1, SMARCA2, SMARCA4, IGHE, SIGMAR1, CD3E, CD2, CD19, MS4A1, CD33, CD38, CD52, CD80, CD86, CTLA4, PDCD1, NCAM1, F5, F8, SERPINC1, FXYD2, IL1B, TNF, VEGFA, NPC1L1, TUBA1A, TUBA4A, TUBB, TUBB3, TUBB4B, and TUBB8.

Alternative Microdrop Compositions.

Further provided herein are alternative microdrop compositions that encompass entities in addition to or alternative to the secretory entities and target entities described herein. The additional or alternative entities are also suspended in the limited permeability material.

In certain embodiments, gel microdrop compositions are provided that comprise a limited permeability material, a first secretory entity that secretes a targeting moiety into the limited permeability material, and a second secretory entity that secretes a target moiety into the limited permeability material. Preferably, the first and the second secretory entity are not the same, i.e. are distinct. Both secretory entities are suspended in the limited permeability material. The limited permeability material is substantially impermeable for the secretory entities. While the limited permeability material is permeable for both the secreted targeting moiety and the secreted target moiety, it is substantially impermeable for a binding complex comprising the targeting moiety and the target moiety. Thus, unbound moieties can be removed by washes while bound entities cannot. Specific binding of the targeting moiety may then be visualized using a detection entity against the targeting moiety as described herein and the microdrop may be selected, e.g. by FACS. Preferably, the secretory entities are cellular entities. This set up is particularly suitable for the detection of interactions between a secreted antigen and a secreted antibody, between a secreted receptor and a secreted ligand, between a secreted enzyme and a secreted substrate, and between a apoenzyme and a cofactor.

In certain embodiments, gel microdrop compositions are provided that comprise a limited permeability material, a first binding entity comprising a targeting moiety, and a second binding entity comprising a target moiety. Preferably, the binding antities are not the same, i.e. are distinct. Both binding entities are suspended in the limited permeability material which is substantially impermeable for both binding entities. Binding of the targeting moiety of the first binding entity to the target moiety of the second binding entity may cause a phenotypic change in one or both of the binding entities that may be detected by a detection entity as described herein. Either one of the binding entities may be cellular or non-cellular but not both entities.

In certain embodiments, gel microdrop compositions are provided that comprise a limited permeability material, a target entity comprising a detectable moiety, and a capture entity capable of engulfing the target entity. Both the target entity and the capture entity are suspended in the limited permeability material. The limited permeability material is substantially impermeable for the capture entity. Optionally, the limited permeability material is permeable for the target entity. Optionally, the target entity is a non-cellular entity, such as a bead. Alternatively, the limited permeability material is substantially impermeable for the target entity, such as a cellular entity. The interaction between the target entity and the capture entity can be detected, e.g., if the engulfment of the target entity by the capture entity, e.g. by phagocytosis, receptor-mediated endocytosis, or pinocytosis, changes a detectable characteristic of the detectable moiety. In a non-limiting example, the change in the detectable characteristic is a detectable change in the wavelength of light emitted from the detectable moiety when it is excited.

Methods to Produce Compositions of Targeting Moieties.

Methods are provided to isolate and purify high affinity targeting moieties that are identified using the screening methods described herein. In certain embodiments, methods are provided that comprise the steps of a) making or providing a library of targeting moieties comprising a plurality of microdrops as described herein, b) incubating the microdrops for a time sufficient to allow secretion and binding of the targeting moiety, c) removing any unbound targeting moiety, e.g. by washing the microdrop, d) contacting the microdrop with a detection entity comprising a detectable moiety, wherein the detection moiety is capable of binding to the targeting moiety, e) removing any non-bound detection moiety, e.g. by washing the microdrop, f) selecting a microdrop for which the detectable moiety is detected, e.g. by FACS or magnetic bead sorting, wherein if the detectable moiety is detected, the targeting moiety has affinity to the target moiety, g) collecting the selected microdrop, h) isolating the secretory entity that secretes the targeting moiety with affinity to the target moiety, and repeating steps (b) to (h) with the isolated secretory entity from step (h), and progressively selecting the microdrops with the highest signal for the detectable moiety in (f), wherein upon repetition a targeting moiety with high affinity to a target moiety is identified from the library of targeting moieties. The high affinity targeting moiety is then isolated by isolating the secretory entity that secretes the high affinity targeting moiety, propagating the isolated secretory entity, and isolating the high affinity targeting moiety from the propagated secretory entities.

Optionally, the screen may be performed by including a step detecting a phenotypic change. For example, by a) contacting the microdrop with a first and a second detection entity comprising a detectable moiety, wherein the first detection entity is capable of binding to the targeting moiety, and the second detection entity is capable of binding to the target entity upon a phenotypic change in the target entity, b) removing a first detection entity not bound to a targeting moiety, and removing a second detection entity not bound to a target entity, and c) selecting a microdrop for which the detectable moiety of the first and the second detection entity is detected, wherein if the first detectable moiety is detected, the targeting moiety has affinity to the target moiety, and if the second detectable moiety is detected, the targeting moiety induces a phenotypic change in the target entity.

The isolated and/or purified targeting moieties may then be packaged and preserved, e.g. by dissolving them in a preservative or by cryo-preservation methods such as freeze-drying.

### EXAMPLES

The following examples are offered by way of illustration and not by way of limitation.

### Example 1

### Generation of Yeast Expression Material

The yeast strain JAC200 which has been engineered for high-fidelity expression of IgG antibodies is transformed with an antibody expression library of 10⁹ in size. The library is a naïve antibody library created by combining CDR diversity directly from naïve human IgM and IgD expressing lymph cells with germline framework and constant region sequence. Alternatively, an immune library in which lymphocytes that have been raised in response to immunization with a particular target or exposure to a particular disease is be used. Other commercially derived antibody libraries are available such as Morphosys' HuCAL libraries, Dyax's and Adimab's antibody libraries, and antibody libraries derived from immunization of humanized or wild-type mice, rats, rabbits, birds, etc. Other libraries of proteinaceous binding scaffolds are also used, such as libraries of diversified fibronectin, DARPINs, or antibody fragments. Libraries of enzymes which will be selected for improved functionality are also constructed and expressed with the yeast platform. The libraries are transformed by electroporation or lithium acetate heat shock using protocols that are well known in the art. The polypeptide libraries are expressed from yeast vectors that contain a galactose-inducible, copper inducible, constitutive (such as ADH1, CYC1, GPD), glucose-repressible, doxycycline/tetracycline-repressible, doxycycline/tetracycline-induced promoters which are well-described in the art. The expression of soluble protein is undertaken in yeast media or mammalian media or a modified version of either. Expressed protein is measured by Western blot, ELISA, activity assay, or other means which are well described in the art.

### Example 2

### Generation of Mammalian Cells Expressing Target Antigen

Target antigen is expressed on mammalian cells by using cell lines that natively express the target on their surface. Such cell lines include tumor cell lines that possess tumor markers that are of interest. These natively expressing cell lines are cultured and maintained using methods that are well described in the art. If there is no natively expressing cell line, or the cell line expresses the target in low amount, the target is artificially overexpressed using a variety of mammalian expression vectors and methods that are well-described in the art, such as vectors for transient transfection or lentiviral systems for stable transfection. This overexpression is performed in a commonly used cell line, such as HEK293 or CHO and culturing the cells under such conditions in which targets are expressed. Some methods providing for membrane protein expression are readily available, and they include, but are not limited to, Life Technologies' TANGO ASSAY CELL LINES which use a beta-arrestin/TEV protesase fusion to yield a fluorescent reporter (GFP or a beta-lactamase activated reporter) of beta-arrestin recruitment to a GPCR fused to a transcription factor. Other options include the GENEBLAZER cell lines and vectors which measure membrane protein activity through the transcription and activity of a beta-lactamase enzyme. Cell lines with reporter activity are particularly useful in the high-throughput analysis of activity from agonistic or antagonistic antibodies.

In addition to cell lines, cell lysate or whole tissue is used to present the target moiety. The tissue is derived from a tumor cell line or the tissue around a tumor. The tissue is alternatively derived from samples containing multiple cells types. The tissue is extracted and homogenized using methods well described in the art. Depending on how the homogenization is done, the sample provides a pool of individual cells containing many cell types from a diseased source, a heterogeneous population of cells that interact with each other, and intracellular material that is used for target presentation. The use of intracellular material allows the discovery of antibodies against intracellular proteins. Immobilization of material from these varying sources is performed by using various bead-labeling methods (such as the DYNAL Epoxy bead labeling systems) to provide beads that have lysate covalently attached to them. In this setup, the beads represent the target entity that presents the target moiety and are used to keep the target moieties (the intracellular/lysate/cellular debris) inside the limited permeability material as the bead is not permeable to the limited permeability material.

### Example 3

### Generation of Permeable Material

Methods for encapsulating cells in semi-permeable membranes are well described in the art, see, e.g., Selimovic S., et. al., “Microscale Strategies for Generating Cell-Encapsulating Hydrogels”, Polymers (2012) 4: 1554-1579. For example, PEG-diacrylate is caused to cross-link in aqueous solution by exposing it to visible light in the presence of eosin Y and triethanol amine. Cells embedded in the solution before cross-linking are then incorporated into the gel. Alternatively, dextran is oxidized to form polyaldehyde which is then crosslinked to collagen. The gels are alternatively enzymatically constructed through the use of tyrosinase, Factor XIII, or transglutaminase in the presence of polypeptides. Gels consisting of alginate are crosslinked with the addition of calcium; poly-vinyl-alcohol gels are crosslinked by the addition of maleic acid. Additionally, cells are suspended in liquid agarose which is then gelled by a decrease in temperature (Kumacheva, E., et. al., “High-throughput combinatorial cell co-culture using microfluidics”, Integrative Biology (2011) 3: 653-662). Cells are also seeded in a “slab” of matrix, if desired and then turned into particles through agitation (such as vortexing), sonication, or other homogenization techniques.

Another approach is to use microfluidics to seed the cells into gel droplets directly. Generally speaking, this method uses an aqueous gel precursor (unsolidified) containing the target entity and secretory entity in conjunction with an immiscible organic phase containing a surfactant and a droplet generating device such as a T-junction, flow-focusing device, co-axial capillaries, or a micro-nozzle cross-flow system. By varying the flow rate of the aqueous phase or phases relative to the immiscible phase, droplets of different sizes are formed. By aligning this droplet forming process with the introduction of a cell (target entity and/or secretory entity), cells are embedded within the aqueous droplet which is later polymerized through the action of a polymerization agent, additional reagent, enzyme, or change in temperature or viscosity. For example, liquid agarose maintained at 37° C. is used to encapsulate two different cell suspensions by flowing the cells through a T-junction droplet generator resulting in the encapsulation of cell-loaded microdroplets within a mineral oil/3% Span-80 continuous phase (Kumecheva et. al. (2011)). After encapsulation the temperature is lowered to 2° C. causing the gelling of the agarose. The agarose microdroplets are then analyzed by flow-cytometry. Yeast and mammalian cells are alternatively encapsulated in alginate through the use of a T-junction that provides for the mixing and subsequent droplet formation through the use of a microfluidic platform encompassing separate cell, alginate, calcium chloride, and hexadecane/Span-80 streams (Lee, Chang-Soo, et. al., “Generation of monodisperse alginate microbeads and in situ encapsulation of cell in micdrofluidic device”, Biomed Microdevices (2007); 9: 855-862). Streams containing the cells, alginate, and calcium chloride are fused just prior to the T-junction which joins the aqueous streams with a continuous oil/surfactant (hexadecane/Span) phase such that the microdroplets are formed at the junction before the alginate is completely gelled. Alternatively, a flow-focusing microfluidic device in conjunction with the UV-activated polymer PEGDA is used for encapsulating microdrops (Zhang, X., et. al., “Rapid Monodisperse Microencapsulation of Single Cells, 32^{nd} Annual International Conference of the IEEE EMBS (2010), Buenos Aires, Argentina). Spheres in the aqueous PEGDA phase are encapsulated in droplets in a Fluorinert oil (FC-40) and 1% Irgacure 2959 continuous phase before being exposed to UV light which causes the polymerization of the monomers around the microspheres. Whatever the method used, at the end of the process a gel microdrop is created such that it has a porosity that prevents the escape of both the secretory entity (e.g. the yeast cell) and the target entity (e.g. a mammalian cell), e.g. a porosity of less than about 1 micron is particularly suitable, but enables the free diffusion of nutrients, secreted targeting moiety (e.g. a polypeptide such as an antibody) and detection entities (e.g. fluorescently labeled antibodies), e.g. a porosity of larger than 10 about nanometers. Other size limitations may be imparted depending on the characterization methods. For example, analyzing microdroplets by flow-cytometry requires the microdroplets to fit within a FACS nozzle, which is typically 100 microns in diameter. Typical applicable methods, reagents, experimental parameters, and optimization steps useful for cell encapsulation in hydrogel microdroplets are described in Kumacheva et. al. “Microfluidic Encapsulation of Cells in Polymer Microgels” small (2012), 8:11, 1633-1642 and Khademhosseini (2012).

In addition to the methods described above, other emulsification-based hydrogel microdroplet generation methods are available. In one strategy, a mixture containing cells to be encapsulated (such as a mixture of mammalian and yeast cells) are suspended in an aqueous solution of low-melt agarose at 37° C. A solution of an oil phase mixed with a surfactant (such as mineral oil mixed with Span) is introduced into the aqueous suspension, and the mixture is agitated (by vortexing or sonication) such that emulsified droplets are created. Moving the emulsified agarose to a lower temperature causes the agarose to gel, and the oil layer and surfactant is removed through washing with a hydrophobic liquid. Alternatively, cells are suspend in a PEGDA polymer, the solution is agitated, and the emulsified droplets exposed to UV light to polymerize the gel. Alternatively, non-water soluble calcium carbonate is mixed with alginate and used to suspend cells. The non-soluble nature of the calcium carbonate in aqueous solutions at neutral pH prevents the alginate from gelling. An oil/surfactant phase is added to the suspension, the mixture agitated, and an acid such as acetic acid is added to the suspension which causes the pH to turn acidic, the calcium carbonate to dissolve, and the alginate to polymerize.

The ratio of target entities (e.g. mammalian cells) to secretory entities (e.g. yeast) can be altered by changing the relative concentration of the two entities. Ideally, there is a one to one ratio between the number of target entities (e.g. mammalian cells) and secretory entities (e.g. yeast). However, in some instances microfluidic and culture-size limitations dictate a surplus of secretory entities to target entities (e.g. there may be more yeast cells than mammalian cells) within the droplet. A typical yeast naïve antibody library is 10⁹ in size, but the throughput of microfludic-based droplet formation is typically millions per hour. As such, the secretory entity to target entity (e.g. yeast to mammalian cell) ratio can be as high as 50:1 in the initial selections. However, as the selection process progresses and target binding yeast clones are enriched, the library size shrinks and progressively fewer secretory entities (e.g. yeast cells) are analyzed. Consequently, the ratio of secretory entity to target entity (e.g. yeast to mammalian cell) increases and often reaches a ratio of 1:1 within two or three rounds of selection. Ultimately, with small targeting moiety libraries, it is possible that there are more target cells than secretory cells (e.g. more mammalian cells than yeast) in each droplet, such as perhaps a 2:1 ratio. Ratios of secretory entity to target entity (e.g. yeast to mammalian cell) are alternatively adjusted by modulating the flow rates of streams containing the secretory entities (e.g. yeast) or target entities (e.g. mammalian cells) relative to each other such that one flows faster, and consequently introduces more of that entity type, than the other within a microfluidic device. Alternatively, yeast and mammalian cells can be mixed directly in a desired ratio, and the mixture acts as a reservoir providing one stream of cells (i.e. mixing takes place before entering a microfluidic device rather than mixing within the microfluidic device).

Once the gel particles are sorted, the outlying gel is degraded so that the secretory entities (e.g. yeast cells) can be removed and processed for more selections or further identification. This degradation occurs via enzymatic processes such as the degradation of agarose by agarase, chemical treatment, or an alteration of the temperature which causes the gel to melt. Enzymes that degrade the peptides that cross-link the gel are introduced to solubilize the matrix. Special functional groups such as esters within non-peptide gels such as poly-vinyl-alcohol make the gels chemically degradable. Alternatively, the gel is melted by increasing the temperature above the melting temperature of the respective polymer.

### Example 4

### Induction and Sorting

After the formation of the target entity/secretory entity (e.g. mammalian cell/yeast cell) droplet, the droplet is incubated under conditions that ensure the fidelity of the target cell (e.g. mammalian cell)-expressed target moiety (e.g. a membrane-associated protein) as well as enable the secretion of the secretory cell (e.g. yeast)-produced targeting moiety (e.g. a polypeptide, such as an antibody). These droplets are incubated as emulsions suspended in a continuous oil phase, or the oil/surfactant phase is removed prior to incubation through washes with an additional oil phase for which the surfactant has preferred solubility. In one example, droplets containing yeast and mammalian cells are incubated in a media containing galactose which induces the production of antibody under the regulation of the Gal1/10 promoter. Alternatively, the induction is performed using a different carbon source with the use of a non-carbon-specific promoter such as a constitutive promoter, e.g. ADH1, CYC1, and GPD1, or doxycycline-repressible or inducible promoter which are commercially available. (Partow S, et al. “Characterization of different promoters for designing a new expression vector in Saccharomyces cerevisiae” Yeast 2010; 27: 955-964). Induction is performed under conditions that are optimal for secretion and mammalian cell capture. These conditions include a shaking culture, a plate culture with no shaking, or using a media that has a viscous additive such as polyethylene glycol to slow the diffusion of protein. The induction is performed in yeast media such as YPD (2% glucose, 2% peptone, 1% yeast extract) or commercially available mammalian cell media such as DMEM with or without the addition of fetal bovine serum. The induction media is buffered to acidic, neutral, or basic pH to retain the fidelity of the targeting moiety (e.g. antibody) and the target moiety (e.g. cell-surface protein). The induction takes place at a suitable temperature that ensures the survival of the yeast cell although typically these inductions take place between 15° C. and 37° C. After sufficient time for antibody production, the droplets are washed to remove any unbound targeting moieties (e.g. antibody) and kept on ice. If the incubation is performed while the droplet is encased in an emulsion, the emulsion can be removed by washes with an oil phase. The droplets are then labeled with a fluorophore-labeled anti-human IgG and sorted for human IgG presence by flow-cytometry. The isolated droplets are then melted using one of the methods described herein or otherwise known in the art and expanded for further rounds of selection.

If the desired result of the selection is the isolation of an antibody that induces a cellular response such as apoptosis, then the induction of the cellular response is part of the selection process. To select for apoptosis, the identification of a mammalian cell-localized antibody is paired with the detection of an apoptotic cell. An apoptotic cell could is marked by staining the cell with a DNA stain such as propidium iodide or DAPI for which apoptotic cells are permeable. Droplets that are co-stained with IgG and the DNA stain are selected and isolated because they contain antibodies with both functional and specific binding attributes. Activities for some targets such as GCPRs are reported through the use of engineered cell lines such as Life Technologies' TANGO ASSAY Cell Line.

As an alternative to flow cytometry, magnetic beads labeled with anti-human IgG antibodies are introduced into the droplet. Magnetic beads come in many sizes and a size that is permeable to the gel droplet is selected. Mammalian cells bearing human IgG on their surface are bound by the magnetic bead thus rendering the droplet magnetic and the droplets are sorted by magnetic field. The advantage of using magnets to sort droplets is that the throughput of magnets is much greater (up to 100-fold greater) than the throughput of FACS.

Once droplets containing the cells are sorted, the secretory entities (e.g. yeast cells) are expanded by inoculating the droplets directly into suitable media, such as yeast media. Alternatively, the droplet is dissolved through an enzyme such as agarase, temperature, or chemical treatment which increases the recovery yield of the secretory entities (e.g. yeast cells). The viability of the target entities (e.g. mammalian cells expressing the target moiety, such as a membrane-associated protein) is not of concern as a fresh culture of mammalian cells is used in the subsequent round of selection. Yeast cells are typically expanded in glucose media which suppresses the expression of the protein of interest on a galactose promoter. If a doxycycline-repressible vector is used, the expansion media contains doxycycline. When the library has expanded at least 10-fold from the original sorted cell number, the process is repeated and the enriched library sorted again.

### Example 5

### Characterization of Resulting Interactions

Antibodies isolated by the selection processes described herein are characterized in a number of ways. Structural integrity of the antibody is interrogated through methods well described in the art, such as Western blotting, size-exclusion chromatography, protease susceptibility, and mass spectrometry among others. Antibodies are isolated directly from secreting yeasts or the genes are isolated by methods well described in the art and cloned into a mammalian or bacterial vector, expressed in a different cell type, and then isolated. Antibody binding affinities are determined by surface-plasmon resonance based approaches or titrations of the antibody on the target cell surface which are both methods well-described in the art.

Functionality of an antibody is best determined by studying how the antibody acts in a cell-binding, tissue culture, or in vivo assay. Isolated antibodies are produced in yeast or other cell lines and then used in functional assays that are well-described in the art. Enzymatic characterization is performed by using enzymes secreted and isolated from yeast in assays that are specific to the enzyme.

### Example 6

### Pre-Screening with Mammalian Cells not Expressing Target Antigen

Mammalian cells produce many surface-localized membrane-associated proteins all of which can form potential targets for antibodies from a naïve library. To eliminate non-target specific antibodies that bind to irrelevant targets, the non-target specific antibodies are eliminated. Non-target specific antibodies are eliminated by a selection against antibodies that bind to non-target proteins. To perform this selection, the yeast-expressed naïve library is mixed with mammalian cells in droplets as described herein. The target entities (e.g. the mammalian cells) used in this negative selection do not express the target moiety (e.g. a surface protein) that is chosen as the target for the selection. The mammalian cells used in this negative selection either do not natively express the target moiety on their surface or they have the target moiety artificially repressed through the use of genetic deletion, RNA interference or degradation, or the use of proteomic approaches such as aptamer co-expession and intrabodies. The selection proceeds as described except that droplets that contain targeting moieties (e.g. antibodies) bound to non-target entities (e.g. mammalian cells) are not selected, and droplets that contain secretory entities (e.g. yeast) and non-target entities (e.g. mammalian cells) with no apparent interaction are retained. The negative selection is performed using flow cytometry or magnetic beads as described herein. The output of the negative selection is used as an input to selections to target moieties. An additional method of performing negative selections is to use cell lysate from non-expressing target entities to bind targeting moieties (e.g. antibodies) that are not specific to the target moiety. For this method, cell-lysate conjugated to beads using DYNAL EPDXY technology is used to select for droplets in which yeast-produced antibodies are not retained on the surface of the lysate-bearing bead. Alternatively, lysate is introduced directly into the media itself in the presence of a target-expressing cell (target entity). This approach provides a droplet with soluble non-specific “competitor” that binds to targeting moieties (e.g. antibodies) that are not specific to the target moiety and are later washed away.

### Example 7

### Competition of Multiple Binders

Targeting moieties that are specific to particular epitopes are selected. For example, in cases where a targeting moiety is competitive with a native ligand for a receptor the targeting moiety can be directly selected using this approach. After co-incubation of the secretory entity (e.g. yeast cell) and the target entity (e.g. mammalian cell) in a droplet, the droplet is labeled with native ligand. If the ligand is not competitive with the antibody, it will bind to the receptor and is detectable with an additional anti-ligand antibody. Consequently, there is a signal for the presence of the ligand and the target-bound yeast-secreted antibody (target entity bound, yeast secreted targeting moiety). However, if the ligand is competitive with the antibody, there is only antibody signal, because the ligand is blocked from receptor binding by the antibody. In this way, cells that show signal correlated with antibody binding (such as with a fluorophore-conjugated anti-human IgG antibody) but do not show signal associated with ligand (such as an anti-ligand fluorophore-conjugated antibody) are selected.

### Example 8

### Secreted Antibody Targeting of a Co-Encapsulated Target-Coated Bead

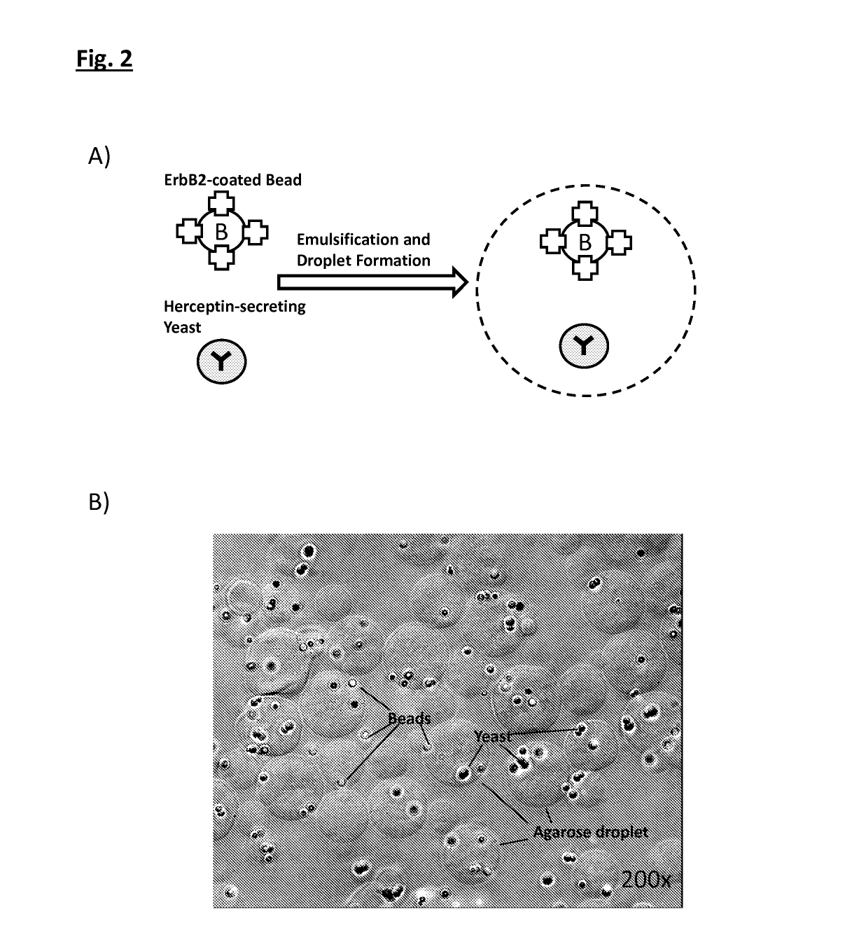

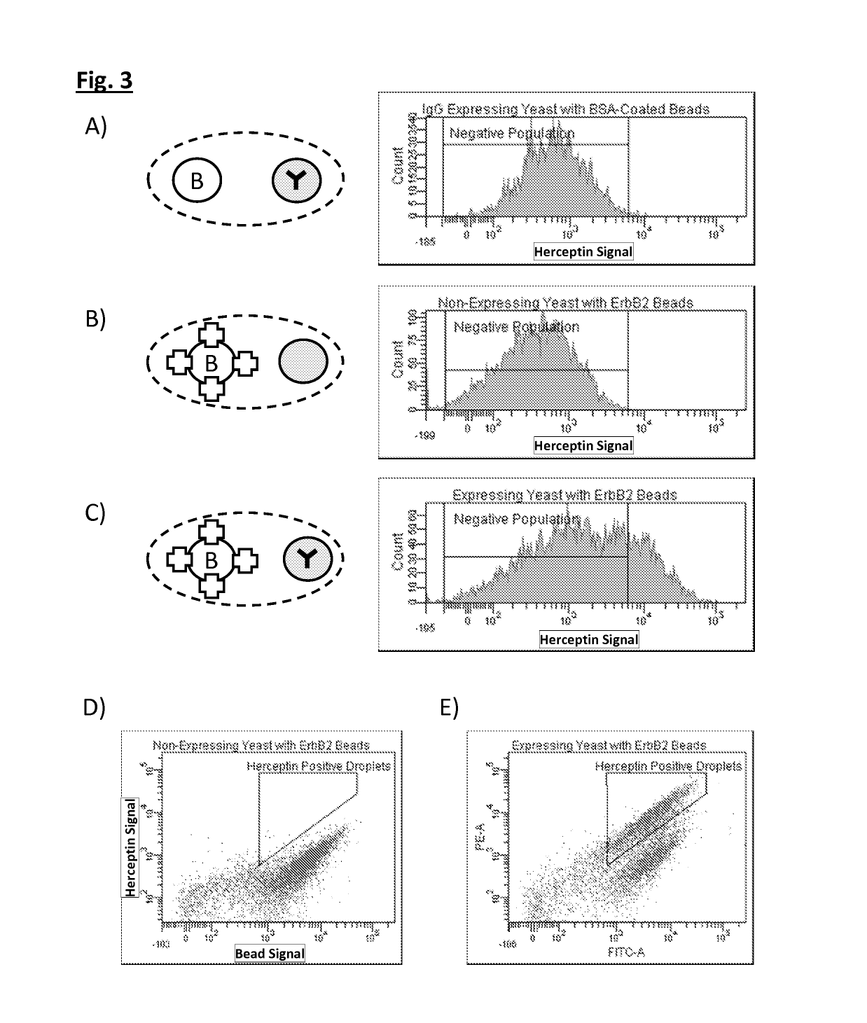

The ability for yeast to secrete an antibody that binds specifically to a co-encapsulated target-coated bead (a surrogate for a co-encapsulated mammalian cell) was demonstrated, FIG. 2 and FIG. 3. 5×10⁵ yeast cells were mixed with 7.5×10⁶ magnetic beads in three samples:
a. yeast expressing a FLAG-tagged Herceptin anti-ErbB2 IgG antibody mixed with 4 micron diameter beads coated with BSA (a protein that does not bind Herceptin) and the fluorophore Alexa488 (FIG. 3A, left panel);
b. yeast not expressing any antibody gene mixed with 4 micron diameter beads coated with ErbB2 (the Herceptin target) and Alexa488 (FIG. 3B, left panel);
c. yeast expressing Herceptin mixed with 4 micron diameter beads expressing ErbB2 and Alexa488 (FIG. 2A, and FIG. 3C, left panel).

The mixture was suspended in 25 μl YPG yeast media (2% galactose substituted for glucose) buffered to pH 7 in phosphate. 25 μl of 2% low-melt agarose dissolved in YPG by heating was cooled to 42° C. and added to the cell/bead mixture which was also maintained at 42° C. 100 μl of mineral oil containing 5% Span-80 was added to the agarose/cell/bead mixture and was immediately vortexed for 60 seconds on a setting of “8” using a VWR-brand vortexer. The resulting emulsion was incubated at room temperature for 16 hours on a rotor to allow the yeast-secreted antibody to bind the co-encapsulated bead (FIG. 2A). Hydrogel-encapsulated beads and yeast were visualized by fluorescence microscopy. Droplet sizes were typically up to 100 microns in diameter, with beads containing both yeast and beads (FIG. 2B, image at 200× magnification). Following incubation, 500 μl of PBS was added to the sample followed by 750 μl of hexadecane. The sample was inverted to mix and then incubated at room temperature for 10 minutes. The “top” hexadecane layer was removed, and the process was repeated three additional times. After the removal of the fourth hexadecane wash, the emulsion was broken, and solid agarose microdroplets were suspended in an aqueous PBS later. The droplets were washed 2 times in 500 μl PBS by centrifugation and resuspension. 100 μl of a 1:1000 dilution of stock biotinylated anti-FLAG antibody (BioM2 from Sigma) diluted in PBS was used to incubate the droplets for 30 minutes at room temperature on a rotor. The droplets were pelleted by centrifugation before being labeled with 100 μl 1:250 dilution of stock streptavidin phycoerythrin (saPE) incubated for 20 minutes at room temperature on a rotor. The droplets were pelleted once more, washed in 500 μl PBS, pelleted and then resuspended in 500 μl PBS. The sample was filtered through a flow cytometry strainer cap before analysis on FACS. For the FACS, droplets were identified by forward scatter and side scatter properties, droplets containing beads were identified by FITC signal, and droplets containing beads bound by the Herceptin antibody were identified by the PE signal (FIGS. 3A, B, C (right panels) and D, E). Only samples that contain both Herceptin-secreting yeast and an ErbB2-coated bead show pronounced PE signal (FIG. 3C); the other samples have a PE peak consistent with no PE staining (FIGS. 3A and B).

### Example 9

### Isolation of Herceptin-Secreting Yeast from Non-Secreting Yeast Through Binding Assay and Flow Cytometry

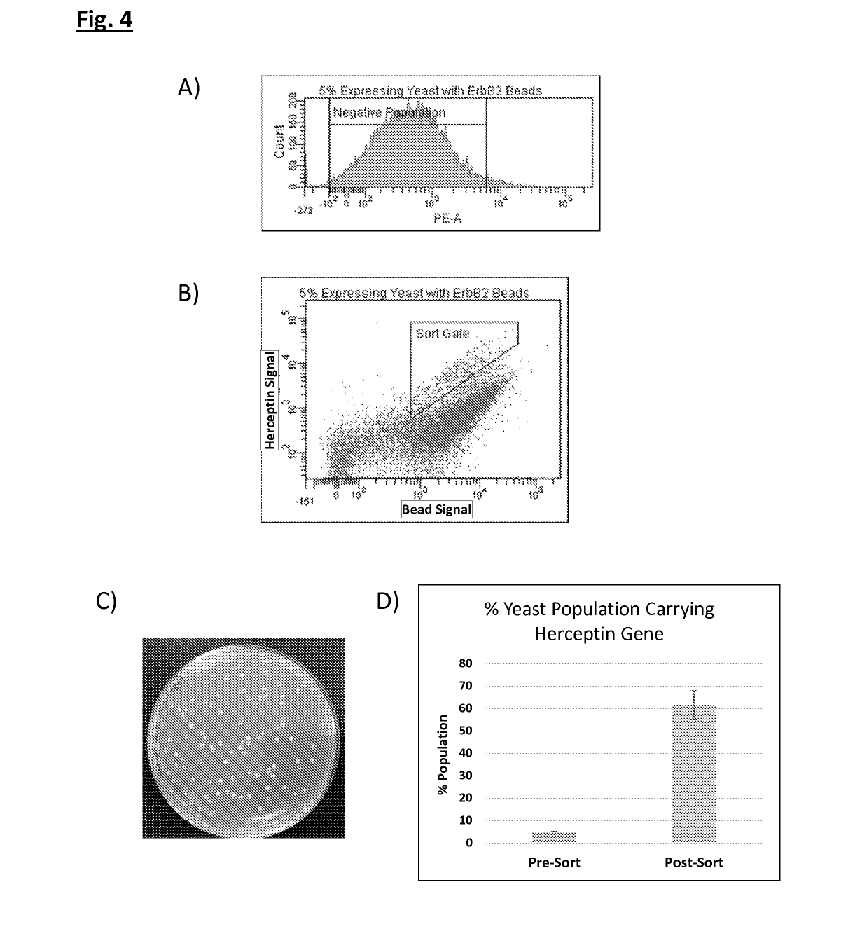

Yeast expressing a target specific antibody were selected from a pool of yeast not bearing the antibody using the encapsulation assay described herein (FIG. 4). A yeast population containing 5% Herceptin-expressing yeast and 95% yeast not expressing an antibody was produced. 5×10⁵ yeast in the mixed population were mixed with 7.5×10⁶ ErbB2 labeled beads also labeled with Alexa488. The yeast were suspended in YPG, mixed with agarose, emulsified, washed, and labeled with BioM2 and streptavidin PE as described in the previous examples. Droplets that were FITC-positive (meaning they contain the target bead) and were most highly stained for PE (roughly the 2%-4% most PE fluorescent of the FITC population) were sorted into YPD media (FIGS. 4A and B). After sorting, agarase was added to a concentration of 20 U/mL and the sample incubated for one hour at 42° C. Experiments showed that treatment with agarase increases the recovery of encapsulated yeast about two-fold. After agarase digestion, the yeast were plated on YPD (media that can grow both Herceptin expressing and non-expressing yeast). After 2 days of growth at 30° C., the plate was replicate plated onto plates lacking tryptophan (media that only Herceptin expressing cells can grow on) (FIG. 4C). Comparing colony growth on YPD and Trp minus plates yielded an enrichment, the percentage of cells that were Herceptin positive post-sort relative to the percentage of cells that were Herceptin-positive in the initial population. This analysis showed that enrichment rates of greater than 10-fold were achieved making this approach suitable for the enrichment of yeast cells expressing target-specific antibodies (FIG. 4D).

### Example 10

### Encapsulation of HEK293 Cells in Agarose

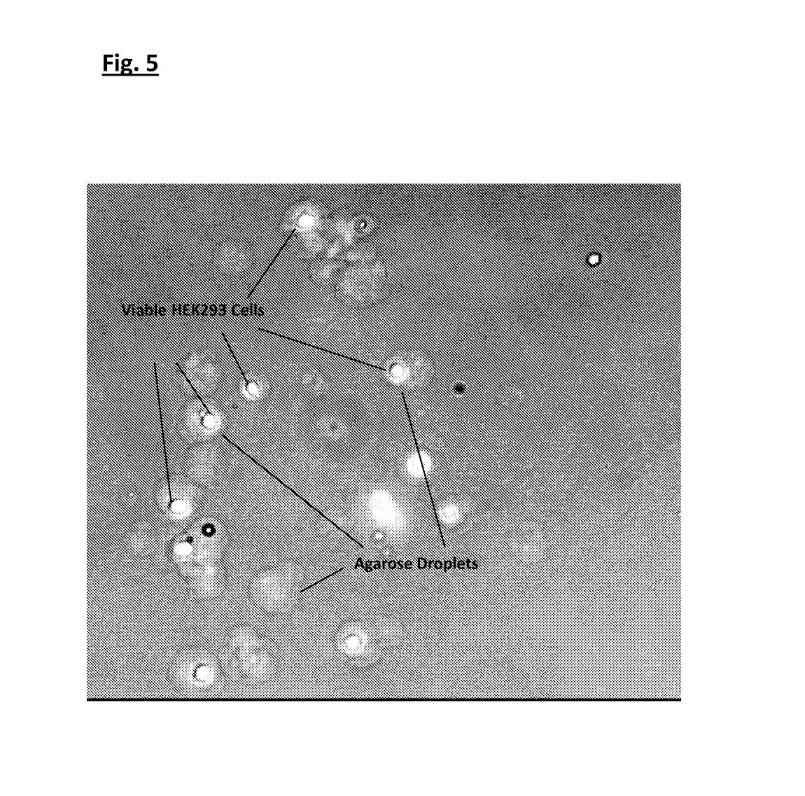

It was shown in the previous example that yeast expressing antibodies specific to a co-encapsulated target-bearing entity were selected from a background of non-secreting cells. It was further determined whether the encapsulation method could preserve the viability of a mammalian cell. 5×10⁵ HEK293 cells were suspended in 25 μl DMEM Eagle media supplemented with 5% fetal bovine serum. This suspension was mixed with 25 μl of 2% low-melt agarose dissolved in DMEM media with FBS. 100 μl mineral oil containing 5% Span-80 was added to the cell suspension, and the mixture was immediately vortexed for 60 seconds on setting of “8” as described herein. The encapsulated cells were incubated in emulsion for 90 minutes before PBS was added and the emulsion removed by washes with hexadecane as described herein. Viability of the encapsulated cells was determined by labeling with Life Technologies' LIVE/DEAD Cell Viability Assays. 100 μl of the stain mixture was incubated for 20 minutes with the droplets. Viability was then assessed by fluorescence microscopy (FIG. 5). Alternatively, encapsulated cells can be identified by FACS. This experiment showed that 80%-90% of the cells were viable demonstrating the usefulness of the assay to identify targeting moieties such as an antibody against live targets.

### Example 11

### Large-Scale Production of Microdroplet Mammalian Complexes

The large-scale production of microdroplet mammalian complexes as could be applicable for the selection of a large library (over one million clones) is also possible by scaling the methods described herein. To scale the method, 600 μl of a mixture containing 1.2×10⁷ Herceptin-secreting yeast cells and 1.2×10⁷ ErbB2 expressing mammalian cells suspended in PBS with 2% low-melt agarose maintained at 42° C. is added to 16 mL of demethylpolysiloxane in a beaker which is being agitated by a 1-inch stir bar at 2000 rpm at 37° C. The resulting emulsion is chilled on ice for 2 minutes before being incubated in the emulsion at 30° C. for 16 hours. After incubation the emulsion is broken and the library selected by FACS as described herein. In place of the stir bar, agitation is accomplished by shaking the emulsion in a high-frequency shaker or a large sonication device. Additionally, larger libraries are created by encapsulating the yeast and mammalian cell using high-throughput microfluidics using a single microfluidic device running at high speed or multiple microfluidic devices running in parallel at lower speeds.

### Example 12

### Selections for CXCR1 Antagonists to Limit Inflammation

CXCR1 is a receptor on neutrophils that binds the cytokine IL-8 (CXCL8) thus promoting an inflammation response by allowing adhesion of neutrophils to endothelial cells in such a manner as to promote their migration toward a site of injury or infection. Binding of IL-8 by the neutrophil receptor causes conformational changes in the adhesion receptors LFA-1 and CR3 which make them more likely to engage adhesion receptors on the endothelium. Antagonizing CXCR1 activity reduces neutrophil activity and consequently reduces aberrant inflammation. To select for CXCR1 antagonists, a plurality of yeast each expressing a differentiated IgG clone is mixed with inactivated neutrophils and encapsulated in microdrops using the methods described herein. After allowing IgG secreted by the yeast to bind to the mammalian cells the microdrops are washed and then stimulated with IL-8. After stimulation with IL-8, the microdrops are labeled with detection entities consisting of antibodies specific to the inactivated LFA-1 conformation. Additionally, antibodies that contain a different fluorophore reactive to the activated LFA-1 conformation are used. FACS selections are then performed by selecting complexes that show binding for the yeast-secreted IgG as well as the antibody specific for the non-active conformation of LFA-1. If an antibody against the activated LFA-1 is also used, those complexes stained with that antibody are disregarded and not isolated.

### Example 13

### Selections for Peptide Activators of the NFκB Pathway

NFκB is a transcription factor involved in activating the expression of pro-inflammatory cytokines. It is most often activated through the stimulation of receptors sensitive to antigens present in the cellular environment. Activation of the Toll-like receptor 4 (TlR-4) by lipopolysaccharide (LPS: a common component of bacterial cell walls) stimulates a pathway that ultimately results in the activation of NFκB and the transcription of multiple pro-inflammatory cytokines such as IL-1, IL6, CXCL8, IL-12, and TNF-α. Selections are performed for peptides that stimulate the TlR-4-mediated pathway. Such peptides are useful in artificially stimulating the inflammatory response in localized areas where an infection is persisting. To perform the selection, a plurality of yeast each expressing a different peptide variant are co-encapsulated with non-activated macrophages in a microdrop. The macrophages recombinantly express a GFP gene under the control of a NFκB response element. Binding of this element by NFκB promotes the production of GFP. After incubating the yeast library and macrophages together in the microdrops, the droplets are washed and labeled with an antibody against an epitope-tag on the secreted peptide. Complexes that are positive for both the presence of the peptide and NFκB activation vis-à-vis GFP expression are selected and the gene for the activating peptide is subsequently isolated. Alternatively, the microdrops contain a neutrophil in addition to the macrophage which are activated by cytokines (specifically IL-8) secreted by macrophages upon macrophage activation. Peptides that stimulate macrophage activation in such a way as to allow the macrophage to stimulate neutrophil activation are selected by detection of neutrophil activation markers (such as a change in LFA-1 conformation described herein) and thus are selected by detecting at phenotypic changes in the neutrophil-an entity that does not interact directly with the peptide.

### OTHER EMBODIMENTS

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Many modifications and other embodiments of the inventions set forth herein will easily come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

All publications and patent applications are herein incorporated by reference to the same extent as if each individual publication or patent application was specifically and individually indicated to be incorporated by reference.

What is claimed is:

1. A gel microdrop composition comprising:
a limited permeability material,
a secretory entity that secretes a targeting moiety into the limited permeability material, and
a target entity comprising a target moiety,
wherein the target entity and the secretory entity both are suspended in the limited permeability material and the limited permeability material is substantially impermeable for both the target entity and the secretory entity but permeable for the secreted targeting moiety, with the proviso that the gel microdrop does not contain a second target entity that is distinct from the first target entity.

2. The composition of claim 1, wherein the microdrop is substantially spherical.

3. The composition of claim 3, wherein the microdrop has a diameter of from about 10 microns to about 100 microns.

4. The composition of claim 1, wherein the microdrop has a volume of from about 4 picoliters to about 4 nanoliters.

5. The composition of claim 1, wherein the microdrop is suspended in a medium, buffer, oil phase, or emulsion.

6. The composition of claim 1, wherein the microdrop is generated by a microfluidics-based method.

7. The composition of claim 1, wherein the limited permeability material comprises a polymer matrix.

8. The composition of claim 7, wherein the polymer matrix is a hydrogel.

9. The composition of claim 8, wherein the hydrogel comprises agarose, carrageenan, alginate, alginate-polylysine, collagen, cellulose, methylcellulose, gelatin, chitosan, extracellular matrix, dextran, starch, inulin, heparin, hyaluronan, fibrin, polyvinyl alcohol, poly(N-vinyl-2-pyrrolidone), polyethylene glycol, poly(hydroxyethyl methacrylate), acrylate polymers and sodium polyacrylate, polydimethyl siloxane, cis-polyisoprene, Puramatrix™, poly-divenylbenzene, polyurethane, or polyacrylamide.

10. The composition of claim 7, wherein the polymer matrix of the limited permeability material has a porosity of from about 10 nm to 5 microns.

11. The composition of claim 1, wherein the secretory entity is a cellular entity.

12. The composition of claim 11, wherein the cellular entity is a yeast cell.

13. The composition of claim 11, wherein the cellular entity is a bacterial cell.

14. The composition of claim 11, wherein the cellular entity is a B cell.

15. The composition of claim 1, wherein the secretory entity is a non-cellular entity.

16. The composition of claim 15, wherein the non-cellular entity comprises a ribosome-mRNA complex.

17. The composition of claim 15, wherein the non-cellular entity comprises cleavable targeting moieties supported on a solid surface that are secreted upon cleavage from the solid surface.

18. The composition of claim 17, wherein the solid surface is a bead.

19. The composition of claim 1, wherein the targeting moiety is a polypeptide.

20. The composition of claim 19, wherein the polypeptide is an antibody or an antibody-like polypeptide.

21. The composition of any one of claims 1, 19 or 20, wherein the secreted targeting moiety specifically binds to the target moiety of the target entity and is retained in the microdrop.

22. The composition of any one of claims 1, 19 or 20, wherein the secreted targeting moiety does not specifically bind to the target moiety of the target entity and is capable of diffusing out of the limited permeability material of the microdrop.

23. The composition of claim 1, wherein the target entity is a cellular entity.

24. The composition of claim 23, wherein the cellular entity is a mammalian cell.

25. The composition of claim 23, wherein the cellular entity is a vertebrate cell.

26. The composition of claim 23, wherein the cellular entity is an invertebrate cell.

27. The composition of claim 24, wherein the mammalian cell is a human cell.

28. The composition of claim 27, wherein the human cell is a healthy or normal cell.

29. The composition of claim 27, wherein the human cell is a neoplastic or atypical cell.

30. The composition of claim 27, wherein the human cell is a cell line.

31. The composition of claim 1, wherein the target entity is a non-cellular entity.

32. The composition of claim 31, wherein the target entity comprises target moieties supported on a solid surface.

33. The composition of claim 32, wherein the solid surface is a bead.

34. The composition of claim 1, wherein the target moiety is an antigen.

35. The composition of claim 34, wherein the target moiety antigen is a cell membrane-associated polypeptide.

36. The composition of claim 35, wherein the cell-membrane associated polypeptide is an ion channel protein, a transporter protein, or a G protein coupled receptor (GPCR).

37. The composition of claim 36, wherein the GPCR is selected from the group consisting of: C3aR, C5aR, FPRL, CXCR4, CCR4, CCR5, CCR2, CCR9, CCR8, GCG-R, GLP-1R, VPAC-1, LGR5, CRTH2, CXCR3, MLNR, ADRA2C, OPRL1, DRD2, HCRTR1, HCRTR2, EDNRA, P2RY12, PTGER4, LTBR4, OXTR, PTGFR, NPY2R, CXCR2, MTNR1B, TACR2, CX3CR1, HTR1F, HTR6, NPSR, SSTR4, SSTR5, SQPR2, PTGER2, SSTR2, CHRM2, CHRM4, ADRB1, ADRB2, SSTR3, GiPR, PTH1R, S1P3, CRTH2, CXCR1, CXCR6, GLP1R, LPAR2, P2RY2, and VIPR1.

38. The composition of any one of claims 34-37, wherein the cell-membrane associated polypeptide is a full-length form.

39. The composition of any one of claims 34-37, wherein the cell-membrane associated polypeptide is not an antigenic fragment of the full length protein.

40. The composition of claim 1, wherein the microdrop contains secretory entities and target entities in a ratio of from about 10:1 to about 1:5.

41. The composition of claim 1, wherein the microdrop contains secretory entities and target entities in a ratio of about 1:1.

42. The composition of claim 1, wherein the microdrop contains secretory entities and target entities in a ratio of about 2:1.

43. The composition of claim 1, wherein the microdrop contains secretory entities and target entities in a ratio of about 5:1.

44. The composition of claim 1, wherein the microdrop contains a single secretory entity and a single target entity.

45. A library of targeting moieties comprising a plurality of microdrops of any one of claims 1-44, wherein the plurality of microdrops comprises a plurality of distinct targeting moieties secreted by a plurality of secretory entities.

46. The library of claim 45, wherein the plurality of microdrops comprises a single target entity comprising a single target moiety.

47. The library of claim 45 or 46, wherein the plurality of secretory entities is a library of yeast expressing and secreting a plurality of targeting moieties.

48. The library of any one of claims 45-47, wherein the targeting moieties are antibody polypeptides or antibody-like polypeptides.

49. The library of claim 48, wherein the library size is from about 10³ clones to about 10¹⁰ clones.

50. The library of claim 48, wherein the library size is from about 10⁶ clones to about 10⁹ clones.

51. The library of any one of claims 45-50, wherein the target entity is a mammalian cell.

52. The library of any one of claims 45-51, wherein the target moiety antigen is a cell membrane-associated polypeptide.

53. A method for detecting a targeting moiety with affinity to a target moiety, the method comprising:
making or providing a gel microdrop composition of any one of claims 1-44,
removing a targeting moiety not bound to a target moiety,
contacting the microdrop with a detection entity comprising a detectable moiety, wherein the detection moiety is capable of binding to the targeting moiety,
removing a detection moiety not bound to a targeting moiety, and
detecting the detectable moiety,
wherein if the detectable moiety is detected, the targeting moiety has affinity to the target moiety.

54. A method for isolating a targeting moiety with affinity to a target moiety, the method comprising:
making or providing a gel microdrop composition of any one of claims 1-44,
removing a targeting moiety not bound to a target moiety,
contacting the microdrop with a detection entity comprising a detectable moiety, wherein the detection moiety is capable of binding to the targeting moiety,
removing a detection moiety not bound to a targeting moiety,
selecting a microdrop for which the detectable moiety is detected,
wherein if the detectable moiety is detected, the targeting moiety has affinity to the target moiety
collecting the selected microdrop, and
isolating the secretory entity that secretes the targeting moiety with affinity to the target moiety.

55. The method of claim 54, wherein isolating the secretory entity comprises dissolution of the limited permeability material.

56. The method of claim 55, wherein dissolution comprises de-polymerization of the limited permeability material.

57. The method of claim 53 or 54, wherein the detection moiety is an antibody specific for the targeting moiety.

58. The method of claim 53 or 54, wherein detectable moiety is a fluorescent molecule.

59. The method of claim 54, wherein selecting the microdrop is carried out using fluorescent activated cell sorting (FACS).

60. A method of making a gel microdrop composition of any one of claims 1-44, the method comprising:
a) combining:
a monomer capable of forming a limited permeability material upon polymerization,
a secretory entity capable of secreting a targeting moiety, and
a target entity comprising a target moiety,
b) forming droplets of the combination of step (a), and
c) polymerizing the monomers of the droplets formed in step (b) to produce gel microdrops comprising a limited permeability material.

61. The method of claim 60, wherein the polymerization is induced by a temperature change of the ambient temperature of the microdrop.

62. The method of claim 60, wherein the polymerization is induced by contacting the microdrop with an enzyme capable of polymerizing the monomers.

63. The method of claim 60, wherein the polymerization is induced by contacting the microdrop with a chemical polymerization agent capable of polymerizing the monomers.

64. The method of any one of claims 60-63, wherein the droplets are formed using a microfuidic apparatus.

65. A method of making a library of targeting moieties comprising a plurality of microdrops of any one of claims 45-52, the method comprising:
a) combining:
a monomer capable of forming a limited permeability material upon polymerization,
a plurality of secretory entities capable of secreting a targeting moiety, wherein the secretory entities are distinct from one another, and
a plurality of target entities comprising a target moiety, wherein the target entities are substantially the same,
b) forming droplets of the combination of step (a), wherein the majority of formed droplets comprises secretory entities and target entities in a ratio of from about 10:1 to about 1:1,
c) polymerizing the monomers of the droplets formed in step (b) to produce gel microdrops comprising a limited permeability material.

66. The method of claim 65, wherein the polymerization is induced by a temperature change of the ambient temperature of the microdrop.

67. The method of claim 65, wherein the polymerization is induced by contacting the microdrop with an enzyme capable of polymerizing the monomers.

68. The method of claim 65, wherein the polymerization is induced by contacting the microdrop with a chemical polymerization agent capable of polymerizing the monomers.

69. The method of claim 65, wherein the polymerization is induced by contacting the microdrop with photons of light.

70. The method of any one of claims 65-69, wherein the droplets are formed using a microfuidic apparatus.

71. A method for isolating a targeting moiety with high affinity to a target moiety from a library of targeting moieties, the method comprising:
a) making or providing a library of targeting moieties comprising a plurality of microdrops of any one of claims 45-52,
b) removing a targeting moiety not bound to a target moiety,
c) contacting the microdrop with a detection entity comprising a detectable moiety, wherein the detection moiety is capable of binding to the targeting moiety,
d) removing a detection moiety not bound to a targeting moiety,
e) selecting a microdrop for which the detectable moiety is detected, wherein if the detectable moiety is detected, the targeting moiety has affinity to the target moiety,
f) collecting the selected microdrop,
g) isolating the secretory entity that secretes the targeting moiety with affinity to the target moiety, and
h) repeating steps (a) to (g) with the isolated secretory entity from step (g), and progressively selecting the microdrops with the highest signal for the detectable moiety in (e), thereby isolating a targeting moiety with high affinity to a target moiety from a library of targeting moieties.

72. The method of claim 71, wherein isolating the secretory entity comprises dissolution of the limited permeability material.

73. The method of claim 71, wherein dissolution comprises de-polymerization of the limited permeability material.

74. The method of claim 71, wherein the detection moiety is an antibody specific for the targeting moiety.

75. The method of claim 71, wherein detectable moiety is a fluorescent molecule.

76. The method of claim 71, wherein selecting the microdrop is carried out using fluorescent activated cell sorting (FACS).

77. The method of claim 71, wherein the majority of the plurality of microdrops comprises secretory entities and target entities in a ratio of from about 10:1 to about 1:1.

78. A method for identifying a targeting moiety from a library of targeting moieties, the method comprising:
a) making or providing a library of targeting moieties comprising a plurality of microdrops of any one of claims 45-52,
b) removing a targeting moiety not bound to a target moiety,
c) contacting the microdrop with a first and a second detection entity comprising a detectable moiety, wherein the first detection entity is capable of binding to the targeting moiety, and the second detection entity is capable of binding to the target entity upon a phenotypic change in the target entity,
d) removing a first detection entity not bound to a targeting moiety, and removing a second detection entity not bound to a target entity,
e) selecting a microdrop for which the detectable moiety of the first and the second detection entity is detected, wherein if the first detectable moiety is detected, the targeting moiety has affinity to the target moiety, and if the second detectable moiety is detected, the targeting moiety induces a phenotypic change in the target entity.

79. The method of claim 78 further comprising:
f) collecting the selected microdrop,
g) isolating the secretory entity that secretes the targeting moiety.

80. The method of claim 79, wherein the phenotypic change in the target entity induced by the targeting moiety is apoptosis, a change in the proteome, a change in the metabolome, a change in the epigenome, or a change in the transcriptome.

81. The method of claim 80, wherein phenotypic change is apoptosis and the second detection entity is DAPI stain, ethidium bromide stain or propidium iodide stain.

82. A mammalian cell complex comprising a mammalian cell, a secretory entity and a limited permeability material, wherein the mammalian cell and the secretory entity are present in and not substantially capable of permeating through the limited permeability material, and wherein the secretory entity is capable of secreting a targeting polypeptide.

83. The cell complex of claim 82, wherein the mammalian cell comprises a target antigen and wherein the targeting polypeptide comprises an antibody or antibody-like polypeptide.

84. The cell complex of claim 83, wherein the antibody or antibody-like polypeptide are capable of permeating through the limited permeability material.

85. The cell complex of claim 83, wherein the antibody or antibody-like polypeptide are capable of specifically binding the target antigen.

86. The cell complex of claim 83, wherein the antibody or antibody-like polypeptide are capable of specifically binding an antigen other than the target antigen.

87. The cell complex of claim 83, wherein the antibody or antibody-like polypeptide comprise a detectable moiety.

88. The cell complex of claim 87, wherein the detectable moiety is fluorescent.

89. The cell complex of claim 83, further comprising a detection agent, wherein the detection agent comprises a detectable moiety, and wherein the detection agent is capable of specifically binding to the antibody or antibody-like polypeptide.

90. The cell complex of claim 89, wherein the antibody or antibody-like polypeptide comprises a detection tag, and wherein the detection agent is capable of binding to the detection tag.

91. The cell complex of claim 83, wherein the antibody or antibody-like polypeptide comprise a separation moiety.

92. The cell complex of claim 91, wherein the separation moiety is magnetic or capable being bound by a magnet.

93. The cell complex of claim 91, further comprising a separation agent, wherein the separation agent comprises a separation moiety, and wherein the separation agent is capable of specifically binding to the antibody or antibody-like polypeptide.

94. The cell complex of claim 93, wherein the separation agent comprises an antibody or antibody-like polypeptide and wherein the separation moiety comprises a magnetic particle.

95. The cell complex of claim 83, wherein the antibody or antibody-like polypeptide comprise a secretion leader peptide.

96. The cell complex of claim 83, wherein the antibody or antibody-like polypeptide is encoded by a nucleic acid sequence encoding a secretion leader peptide.

97. The cell complex of claim 82, wherein the mammalian cell comprises a cell surface receptor and wherein the targeting polypeptide comprises a ligand capable of specifically binding to the cell surface receptor.

98. The cell complex of claim 82, wherein the mammalian cell comprises an antigen and wherein the targeting polypeptide comprises an antibody or antibody-like polypeptide.

99. The cell complex of claim 82, wherein the mammalian cell comprises a substrate and wherein the targeting polypeptide comprises an enzyme capable of acting upon the substrate.

100. The cell complex of claim 82, wherein the mammalian cell comprises an enzyme and wherein the targeting polypeptide comprises a substrate capable of being acted upon by the enzyme.

101. The cell complex of claim 82, wherein the targeting polypeptide comprises a cell penetrating polypeptide, and wherein the mammalian cell is detectably modified upon penetration by the cell penetrating polypeptide.

102. The cell complex of claim 82, wherein the mammalian cell and the secretory entity are present in the limited permeability material at a ratio from about 5:1.

103. The cell complex of claim 82, wherein the mammalian cell and the secretory entity are present in the limited permeability material at a ratio from about 1:1.

104. The cell complex of claim 82, wherein the mammalian cell and the secretory entity are present in the limited permeability material at a ratio from about 1:10.

105. The cell complex of claim 82, wherein the secretory entity comprises a bacterial cell.

106. The cell complex of claim 82, wherein the secretory entity comprises a yeast cell.

107. The cell complex of claim 82, wherein the secretory entity comprises a ribosome-mRNA complex.

108. The cell complex of claim 82, wherein the limited permeability material has a porosity of from about 10 nm to about 1000 nm.

109. The cell complex of claim 82, wherein the limited permeability material is substantially spherical and has a diameter less than about 100 microns.

110. The cell complex of claim 82, wherein the secretory entity comprises a plant cell.

111. The cell complex of claim 82, wherein the secretory entity comprises a mammalian cell.

112. The cell complex of claim 82, wherein all secretory entities present in the limited permeability material are capable of secreting the same targeting polypeptide.

113. The cell complex of claim 82, wherein the targeting polypeptide is capable of binding to the mammalian cell, and wherein such binding causes the transduction of a cell signal.

114. A library comprising a plurality of cell complexes of claim 82.

115. The library of claim 114, further comprising a retention device capable of individually retaining the cell complexes present in the library.

116. The library of claim 115, wherein the retention device comprises a solid or semi-solid support material.

117. The library of claim 115, wherein the retention device comprises a liquid or gel support material.

118. A method of displaying a secreted engineered protein complex on a mammalian cell, the method comprising:
a) providing a mammalian cell complex comprising a mammalian cell, a secretory entity and a limited permeability material, wherein the mammalian cell and the secretory entity are present in and not substantially capable of permeating through the limited permeability material, wherein the secretory entity comprises a first nucleic acid, and
b) incubating the mammalian cell complex under conditions sufficient for expressing by the secretory entity a engineered protein encoded by the first nucleic acid, wherein the engineered protein is secreted by the secretory entity, and wherein the engineered protein binds to a binding moiety present on the mammalian cell, thereby forming a secreted engineered protein complex on the mammalian cell.

119. A method of selecting a mammalian cell, the method comprising:
a) providing a mammalian cell complex comprising a mammalian cell, a secretory entity and a limited permeability material, wherein the mammalian cell expresses a target polypeptide, wherein the target polypeptide is located at the plasma membrane of the mammalian cell, wherein the mammalian cell and the secretory entity are present in and not substantially capable of permeating through the limited permeability material, wherein the secretory entity comprises a first nucleic acid,
b) incubating the mammalian cell complex under conditions sufficient for expressing by the secretory entity a engineered protein encoded by the first nucleic acid, wherein the engineered protein is secreted by the secretory entity, and wherein the engineered protein binds to the target polypeptide on the mammalian cell, and
c) detecting the engineered protein bound to the target polypeptide, thereby selecting the mammalian cell.

120. A method of screening an engineered protein, the method comprising:
a) providing a plurality of mammalian cell complexes, wherein each mammalian cell complex independently comprises a mammalian cell, a secretory entity and a limited permeability material, wherein the mammalian cell expresses a target polypeptide, wherein the target polypeptide is located at the plasma membrane of the mammalian cell, wherein the mammalian cell and the secretory entity are present in and not substantially capable of permeating through the limited permeability material, wherein the secretory entity comprises a first nucleic acid,
b) incubating the plurality of mammalian cell complexes under conditions sufficient for expressing by the secretory entity a engineered protein encoded by the first nucleic acid, wherein the engineered protein is secreted by the secretory entity, and wherein in at least one mammalian cell complex the engineered protein binds to the target polypeptide on the mammalian cell,
c) detecting the engineered protein bound to the target polypeptide, and
d) identifying the detected engineered protein, thereby screening the engineered protein.

121. The method of claim 119, comprising providing at least about 1×10⁴ mammalian cell complexes.

122. The method of claim 119, wherein each engineered protein comprises an antibody, wherein at least about 1×10⁵ unique engineered proteins are present in the plurality of mammalian cell complexes.

123. A method of sorting mammalian cells, the method comprising:
a) providing a first mammalian cell complex comprising a mammalian cell, a secretory entity and a limited permeability material, wherein the mammalian cell expresses a target polypeptide, wherein the target polypeptide is located at the plasma membrane of the mammalian cell, wherein the mammalian cell and the secretory entity are present in and not substantially capable of permeating through the limited permeability material, wherein the secretory entity comprises a first nucleic acid,
b) incubating the mammalian cell complex under conditions sufficient for expressing by the secretory entity a engineered protein encoded by the first nucleic acid, wherein the engineered protein is secreted by the secretory entity, and wherein the engineered protein binds to the target polypeptide on the mammalian cell, and
c) detecting the engineered protein bound to the target polypeptide, thereby selecting the mammalian cell.

124. A gel microdrop composition comprising:
a limited permeability material,
a first secretory entity that secretes a targeting moiety into the limited permeability material, and
a second secretory entity that secretes a target moiety into the limited permeability material,
wherein the first and the second secretory entity are not the same and both are suspended in the limited permeability material, wherein the limited permeability material is substantially impermeable for both secretory entities, and wherein the limited permeability material is permeable for both the secreted targeting moiety and the secreted target moiety, but substantially impermeable for a binding complex comprising the targeting moiety and the target moiety.

125. The microdrop of claim 124, wherein the first and the second secretory entities are cellular entities.

126. The microdrop of claim 124 or 125, wherein the first secretory entity secretes an antigen and the second secretory entity secretes an antibody.

127. The microdrop of claim 124 or 125, wherein the first secretory entity secretes a receptor molecule and the second secretory entity secretes a ligand.

128. The microdrop of claim 124 or 125, wherein the first secretory entity secretes an enzyme and the second secretory entity secretes a substrate.

129. The microdrop of claim 124 or 125, wherein the first secretory entity secretes an apoenzyme and the second secretory entity secretes a cofactor.

130. A gel microdrop composition comprising:
a limited permeability material,
a first binding entity comprising a targeting moiety, and
a second binding entity comprising a target moiety,
wherein the first and the second binding entity are not the same and both are suspended in the limited permeability material, wherein the limited permeability material is substantially impermeable for both binding entities, and wherein binding of the targeting moiety of the first binding entity to the target moiety of the second binding entity causes a phenotypic change in one or both of the binding entities.

131. The microdrop of claim 130, wherein the first and second binding entities are cellular.

132. The microdrop of claim 130, wherein the first or the second binding entity is cellular and the other entity is non-cellular.

133. The microdrop of any one of claims 130-132, wherein the phenotypic change is apoptosis.

134. The microdrop of any one of claims 130-132, wherein the phenotypic change is a change in the proteome, the metabolome, the epigenome, or the transcriptome.

135. A gel microdrop composition comprising:
a limited permeability material,
a target entity comprising a detectable moiety, and
a capture entity capable of engulfing the target entity,
wherein the target entity and the capture entity both are suspended in the limited permeability material, and wherein the limited permeability material is substantially impermeable for the capture entity.

136. The microdrop of claim 135, wherein the limited permeability material is permeable for the target entity.

137. The microdrop of claim 136, wherein the target entity is a non-cellular entity.

138. The microdrop of claim 137, wherein the non-cellular target entity is a bead.

139. The microdrop of claim 135, wherein the limited permeability material is substantially impermeable for the target entity.

140. The microdrop of claim 139, wherein the target entity is a cellular entity.

141. The microdrop of claim 140, wherein engulfment of the target entity by the capture entity changes a detectable characteristic of the detectable moiety.

142. The microdrop of claim 141, wherein the change in the detectable characteristic is a detectable change in the wavelength of light emitted from the detectable moiety when it is excited.

143. The microdrop of any one of claims 135-142, wherein the capture entity is a cellular entity.

144. The microdrop of claim 143, wherein the cellular entity is a macrophage.

145. A method for producing a targeting moiety with high affinity to a target moiety from a library of targeting moieties, the method comprising:
a) making or providing a library of targeting moieties comprising a plurality of microdrops of any one of claims 45-52,
b) removing a targeting moiety not bound to a target moiety,
c) contacting the microdrop with a detection entity comprising a detectable moiety, wherein the detection moiety is capable of binding to the targeting moiety,
d) removing a detection moiety not bound to a targeting moiety,
e) selecting a microdrop for which the detectable moiety is detected, wherein if the detectable moiety is detected, the targeting moiety has affinity to the target moiety,
f) collecting the selected microdrop,
g) isolating the secretory entity that secretes the targeting moiety with affinity to the target moiety, and
h) repeating steps (a) to (g) with the isolated secretory entity from step (g), and progressively selecting the microdrops with the highest signal for the detectable moiety in (e), wherein upon repetition a targeting moiety with high affinity to a target moiety is identified from the library of targeting moieties,
i) isolating the secretory entity that secretes the high affinity targeting moiety identified in step (h),
j) propagating the isolated secretory entity from step (i), and
k) isolating the high affinity targeting moiety.

146. A method for producing a targeting moiety from a library of targeting moieties:
a) making or providing a library of targeting moieties comprising a plurality of microdrops of any one of claims 45-52,
b) removing a targeting moiety not bound to a target moiety,
c) contacting the microdrop with a first and a second detection entity comprising a detectable moiety, wherein the first detection entity is capable of binding to the targeting moiety, and the second detection entity is capable of binding to the target entity upon a phenotypic change in the target entity,
d) removing a first detection entity not bound to a targeting moiety, and removing a second detection entity not bound to a target entity,
e) selecting a microdrop for which the detectable moiety of the first and the second detection entity is detected, wherein if the first detectable moiety is detected, the targeting moiety has affinity to the target moiety, and if the second detectable moiety is detected, the targeting moiety induces a phenotypic change in the target entity,
f) collecting the selected microdrop,
g) isolating the secretory entity that secretes the targeting moiety with affinity to the target moiety, and
h) repeating steps (a) to (g) with the isolated secretory entity from step (g), and progressively selecting the microdrops with the highest signal for the detectable moiety in (e), wherein upon repetition a targeting moiety with high affinity to a target moiety is identified from the library of targeting moieties,
i) isolating the secretory entity that secretes the high affinity targeting moiety identified in step (h),
j) propagating the isolated secretory entity from step (i), and
k) isolating the high affinity targeting moiety.

147. The method of claim 145 or 146, further comprising preserving the high affinity targeting moiety.

148. The method of claim 147, wherein preserving comprises dissolving the targeting moiety in a medium comprising a preservative.

149. The method of claim 147, wherein preserving comprises drying the targeting moiety.

150. The method of 149, wherein drying comprises freeze-drying the targeting moiety.
